SEQUENCE LISTING (112 sequences)

SEQ ID NO: 1 - DNA, 1037 bp, Homo sapiens
  source 1..1037: mol_type = genomic DNA
tttgcttctaggaagcagaagactgaggaaatgacttgggcgggtgcatcaatgcggccaaaaaagacacggacacgctcccctgggacctgagctggttcgcagtcttcccaaaggtgccaagcaagcgtcagttcccctcaggcgctccaggttcagtgccttgtgccgagggtctccggtgccttcctagacttctcgggacagtctgaaggggtcaggagcggcgggacagcgcgggaagagcaggcaaggggagacagccggactgcgcctcagtcctccgtgccaagaacaccgtcgcggaggcgcggccagcttcccttggatcggactttccgcccctagggccaggcggcggagcttcagccttgtcccttccccagtttcgggcggcccccagagctgagtaagccgggtggagggagtctgcaaggatttcctgagcgcgatgggcaggaggaggggcaagggcaagagggcgcggagcaaagaccctgaacctgccggggccgcgctcccgggcccgcgtcgccagcacctccccacgcgcgctcggccccgggccacccgccctcgtcggcccccgcccctctccgtagccgcagggaagcgagcctgggaggaagaagagggtaggtggggaggcggatgaggggtgggggaccccttgacgtcaccagaaggaggtgccggggtaggaagtgggctggggaaaggttataaatcgcccccgccctcggctgctcttcatcgaggtccgcgggaggctcggagcgcgccaggcggacactcctctcggctcctccccggcagcggcggcggctcggagcgggctccggggctcgggtgcagcggccagcgggcgcctggcggcgaggattacccggggaagtggttgtctcctggctggagccgcgagacgggcgctcagggcgcggggccggcggcggcgaacaagaggacggactctggcggccgggtcgttggccgcggggagcgcgggcaccgggcgagcaggccgcgtcgcgctcacc

SEQ ID NO: 2 - DNA, 1608 bp, Homo sapiens
  source 1..1608: mol_type = genomic DNA
agctcctcccagcctcaggcccaggaatgggaatctctgtgggtcacacatcagtagggaggtctttcccgatccttttctatgctactccaggagtcaaagcgtctcctgggacttttcagggcgcttcagaagagccctgggcctaaaccagctcaaccaagctgcagggacccagcctcctgagaaaagtgaatgtgagcccggtgcattcagaggagaatgaagccttcacccagaacacactctgggaagatgtcccaggcccagggggagggtttgtactaccagacctaagtcacctaaactgacaccaagtctcatccatcccaaccattccattccgggtcagaggggtcatcgatttaaccagcaaggctgcccatccaacggttgctccctctgctccctggaagggcctcctcgtgggcgttctgtacctacaggtcttgttccgttctgggaactgccagtggtggcaagaggtggagcaacgggtgccagggcagggagaggtgagtctgggagggaagcagaggcaagatccatggggctttagagactttgccaaagcagtgcgactgctcccaggttgttgtcagccgtcaagagtgagtgcacctccctgggcagacttctgctgccccagtgcccaggaataggcaggggtttgccgcaaaatgaatgacacctggcagacaataagctgaagctttcattagcagcttaagctgaggactatctatgcaaccgatactccctgtgtgctccccgggactgcttaatgtgagcccttgtggagcgattggcaccaagaaagcaaggactaagtcagaagttcaagtcccagccttgccacagcctcagggtgccctcgagcacagcaagcctcagttttcccatctgtacaatgagagaggtacacaaggtagactcgaaggctctttgttgccagggccctgtgttcctttgagtgtatgtgcttctcaggcccacagaggtcctttgtgtttcgtatgtgaactgctctctaggaaacccatgtaactgtctgtgtcctggggcacatacatgaggactcatgtgggccgtattgtgtgtttgtgccggggggaggggagaccccagaacaatgtcccccaccccacccccctcctcaataggcggaagccactggcttcctccctttcctgcctcctgcctcctttgtgccagcaagactgagtactggagagagacaggggatgggaaaaatcagtccagctgtccccaggtctgcccttaccataaccttccccccacctcaagtgactcctcccaggccacacccatccccagccttgtgggggccagattggggggcctagaggctcaaaggcagaatgagtcctcccaccccctaccctgccacccctcccacccaagccacctcatttcctcttcctccccagcaccgacccacactgaccaacacaggctgagcagtcaggcccacagcatctgaccccaggcccagctcgtcctggctggcctgggtcggcctctggagtatggtctggcgggtgccccctttcttgctccccatcctcttcttggcttctcatgtgg

SEQ ID NO: 3 - DNA, 2510 bp, Homo sapiens
  source 1..2510: mol_type = genomic DNA
ctagtagcagaaacaaggtcctctggaagagcaactgatgctcttaggtactgaagcatcatcctgccccagagaccactcgcatatgaagcacacatattcagtctgccttacttgtgttaatgattgccagtgtccctctgacctcctagccctgaaaagtgtggcctgaaggtcatttcagagacggggagagctgctcagagaagccaatcggcgagtctaggacacacagacaggatctagtcccagagttcgctagcctaggtgagcgtcccctggccccttataccacttccttctccagcttgcatctaatctgctctggcagaccatcgtgtttcctgtcttcctggcagcctccagcacgctcagtgctactccctgcgcatgcgccctcctcccagtaccttctctgactccagtgggcttggagtgcgaggaggaagggtgaggaaggggtgaaatcaggtattggatccacagggggtctgaagagcactagcctggccttttgggactgaacttctgctatgaagacctccactgccatccctggagtccggggcacatccaaggcttgctgtccatcgtttactgtttacagatgacaacaatgactgtgttcggggcagaaatatccaccagggctagagtacaaaaggagtttgcattgatggccggacaggccctgtccctggcagcctgccagcgctgagtatgagacccagcgggaagtgctaccctggcagacgtgtccactgagtacacagaccaccaaggcaggcagctctcggggaagctgtctatgctgggccagcccaccttgagggcagggaacagaacagattgtggcagagaggaaaatgtggagcttctgtttgttcacagacacacgcactcgcccacgcacgcacgcacgcacgcacgcacgcacgaatgcacgcacgcagtagttgaatgctatggattccgctcagagctgagaacagccccagcgacagttccctggcctctctccttactctgatgtcctcatctgtcttcacatggtctcaggacgctaatactccatcctaatgtacactcctttccctgggcctccgttccagttcagttctcagaggacctggagggagtgattggctacaccaactttgctttcgttcaccaagcccatgtctctacttgggtgtctaatgggcatctccaacattacctaccccaaacagaaaaccctttcttccccccaaccacaccccaccctacccccacagtattttctccatgcccggaaagatctgctctcttatggtccctctttgcctcactgaaaagcaggacaagttggggacttcccaaacttttatgcatgaagaaacccaggcaatttgccaaaaggtacactctgggggtctgtcatttactctgagccagaaccctgaaatttttactaacccatcacataatgaatgaagagaatctttttctttttttttttttttctttttttttggtttttcgagacagggtttctctgtatagccctggctatcctggaacacactctgtagaccaggctggcctcgaactcagaaatccacctgcctctgcctcccgagtgctgggattaaaggcgtgcgccaccacgcctggctgaatgaagagaatcttgacctcatctccccagcctcttggtcctgagggaccctggtctacctactgctttgctgtcttcttagctcttcttacttttttgctgactcagacctatggctatctccattatacagatgaggagactgaggcatggatccctggttggtccatggtcacgtgaagcccatcacccagtatttgtaaagtgagatgggccaggctggtaccttggaactgaaactcacactgccctacctggaagaatctgacaggcaaaatctgctgctgaaagtgattgtctgtcacgtttctcagctgcccgactctgagaactccacagccccctttcgttccaccatactacagagtcgccacggaaagccggctctgtggagaagctgaggtagctgggtttctgtctgggttactctgtccagcgaggaaacaagtaccttagacccactaagcctctgctttctgaactgtaaagtgggggatatgacacctgcctcccagggatggctgaatgctctggcagaagcttagagcccccacagctacccctaggctcacagctcctccgatgagacctagaattgaggtatgagttgaataccccaggcaggtccaaggcttccacgggcccaggctgaccaagctgaggccgcccaccgtagggcttgcctatctgcaggcagctcacaaaggaacaataacaggaaaccatcccgaggggaagtgggccagggccagttggaaaacctgcctccctcccagcctgggtgtggctcccctctcccctcctgaggcaatcaactgtgctctccacaaagctcggccctggacagact

SEQ ID NO: 4 - DNA, 94 bp, Homo sapiens
  source 1..94: mol_type = genomic DNA
gctggagcctcggtagccgttcctcctgcccgctgggcctcccaacgggccctcctcccctccttgcaccggcccttcctggtctttgaataaa

SEQ ID NO: 5 - DNA, 1476 bp, Homo sapiens
  source 1..1476: mol_type = genomic DNA
atggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtg

SEQ ID NO: 6 - DNA, 168 bp, unidentified
  source 1..168: mol_type = genomic DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 7 - DNA, 168 bp, unidentified
  source 1..168: mol_type = genomic DNA
tacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 8 - DNA, 2963 bp, synthetic construct
  misc_feature 1..2963: note = Made in Lab - part of expression cassette
  source 1..2963: mol_type = other DNA
ctctggagacgcgttacatacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttc

SEQ ID NO: 9 - skipped

SEQ ID NO: 10 - DNA, 3414 bp, synthetic construct
  misc_feature 1..3414: note = Made in Lab - part of expression cassette
  source 1..3414: mol_type = other DNA
ctctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttc

SEQ ID NO: 11 - skipped

SEQ ID NO: 12 - DNA, 3409 bp, synthetic construct
  misc_feature 1..3409: note = Made in Lab - part of expression cassette
  source 1..3409: mol_type = other DNA
ctctggagacgcgttacatatttgcttctaggaagcagaagactgaggaaatgacttgggcgggtgcatcaatgcggccaaaaaagacacggacacgctcccctgggacctgagctggttcgcagtcttcccaaaggtgccaagcaagcgtcagttcccctcaggcgctccaggttcagtgccttgtgccgagggtctccggtgccttcctagacttctcgggacagtctgaaggggtcaggagcggcgggacagcgcgggaagagcaggcaaggggagacagccggactgcgcctcagtcctccgtgccaagaacaccgtcgcggaggcgcggccagcttcccttggatcggactttccgcccctagggccaggcggcggagcttcagccttgtcccttccccagtttcgggcggcccccagagctgagtaagccgggtggagggagtctgcaaggatttcctgagcgcgatgggcaggaggaggggcaagggcaagagggcgcggagcaaagaccctgaacctgccggggccgcgctcccgggcccgcgtcgccagcacctccccacgcgcgctcggccccgggccacccgccctcgtcggcccccgcccctctccgtagccgcagggaagcgagcctgggaggaagaagagggtaggtggggaggcggatgaggggtgggggaccccttgacgtcaccagaaggaggtgccggggtaggaagtgggctggggaaaggttataaatcgcccccgccctcggctgctcttcatcgaggtccgcgggaggctcggagcgcgccaggcggacactcctctcggctcctccccggcagcggcggcggctcggagcgggctccggggctcgggtgcagcggccagcgggcgcctggcggcgaggattacccggggaagtggttgtctcctggctggagccgcgagacgggcgctcagggcgcggggccggcggcggcgaacaagaggacggactctggcggccgggtcgttggccgcggggagcgcgggcaccgggcgagcaggccgcgtcgcgctcaccgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttc

SEQ ID NO: 13 - skipped

SEQ ID NO: 14 - DNA, 3980 bp, synthetic construct
  misc_feature 1..3980: note = Made in Lab - part of expression cassette
  source 1..3980: mol_type = other DNA
ctctggagacgcgttacataagctcctcccagcctcaggcccaggaatgggaatctctgtgggtcacacatcagtagggaggtctttcccgatccttttctatgctactccaggagtcaaagcgtctcctgggacttttcagggcgcttcagaagagccctgggcctaaaccagctcaaccaagctgcagggacccagcctcctgagaaaagtgaatgtgagcccggtgcattcagaggagaatgaagccttcacccagaacacactctgggaagatgtcccaggcccagggggagggtttgtactaccagacctaagtcacctaaactgacaccaagtctcatccatcccaaccattccattccgggtcagaggggtcatcgatttaaccagcaaggctgcccatccaacggttgctccctctgctccctggaagggcctcctcgtgggcgttctgtacctacaggtcttgttccgttctgggaactgccagtggtggcaagaggtggagcaacgggtgccagggcagggagaggtgagtctgggagggaagcagaggcaagatccatggggctttagagactttgccaaagcagtgcgactgctcccaggttgttgtcagccgtcaagagtgagtgcacctccctgggcagacttctgctgccccagtgcccaggaataggcaggggtttgccgcaaaatgaatgacacctggcagacaataagctgaagctttcattagcagcttaagctgaggactatctatgcaaccgatactccctgtgtgctccccgggactgcttaatgtgagcccttgtggagcgattggcaccaagaaagcaaggactaagtcagaagttcaagtcccagccttgccacagcctcagggtgccctcgagcacagcaagcctcagttttcccatctgtacaatgagagaggtacacaaggtagactcgaaggctctttgttgccagggccctgtgttcctttgagtgtatgtgcttctcaggcccacagaggtcctttgtgtttcgtatgtgaactgctctctaggaaacccatgtaactgtctgtgtcctggggcacatacatgaggactcatgtgggccgtattgtgtgtttgtgccggggggaggggagaccccagaacaatgtcccccaccccacccccctcctcaataggcggaagccactggcttcctccctttcctgcctcctgcctcctttgtgccagcaagactgagtactggagagagacaggggatgggaaaaatcagtccagctgtccccaggtctgcccttaccataaccttccccccacctcaagtgactcctcccaggccacacccatccccagccttgtgggggccagattggggggcctagaggctcaaaggcagaatgagtcctcccaccccctaccctgccacccctcccacccaagccacctcatttcctcttcctccccagcaccgacccacactgaccaacacaggctgagcagtcaggcccacagcatctgaccccaggcccagctcgtcctggctggcctgggtcggcctctggagtatggtctggcgggtgccccctttcttgctccccatcctcttcttggcttctcatgtgggccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttc

SEQ ID NO: 15 - skipped

SEQ ID NO: 16 - DNA, 4380 bp, synthetic construct
  misc_feature 1..4380: note = Made in Lab - part of expression cassette
  source 1..4380: mol_type = other DNA
ctctggagacgcgttacatactagtagcagaaacaaggtcctctggaagagcaactgatgctcttaggtactgaagcatcatcctgccccagagaccactcgcatatgaagcacacatattcagtctgccttacttgtgttaatgattgccagtgtccctctgacctcctagccctgaaaagtgtggcctgaaggtcatttcagagacggggagagctgctcagagaagccaatcggcgagtctaggacacacagacaggatctagtcccagagttcgctagcctaggtgagcgtcccctggccccttataccacttccttctccagcttgcatctaatctgctctggcagaccatcgtgtttcctgtcttcctggcagcctccagcacgctcagtgctactccctgcgcatgcgccctcctcccagtaccttctctgactccagtgggcttggagtgcgaggaggaagggtgaggaaggggtgaaatcaggtattggatccacagggggtctgaagagcactagcctggccttttgggactgaacttctgctatgaagacctccactgccatccctggagtccggggcacatccaaggcttgctgtccatcgtttactgtttacagatgacaacaatgactgtgttcggggcagaaatatccaccagggctagagtacaaaaggagtttgcattgatggccggacaggccctgtccctggcagcctgccagcgctgagtatgagacccagcgggaagtgctaccctggcagacgtgtccactgagtacacagaccaccaaggcaggcagctctcggggaagctgtctatgctgggccagcccaccttgagggcagggaacagaacagattgtggcagagaggaaaatgtggagcttctgtttgttcacagacacacgcactcgcccacgcacgcacgcacgcacgcacgcacgcacgaatgcacgcacgcagtagttgaatgctatggattccgctcagagctgagaacagccccagcgacagttccctggcctctctccttactctgatgtcctcatctgtcttcacatggtctcaggacgctaatactccatcctaatgtacactcctttccctgggcctccgttccagttcagttctcagaggacctggagggagtgattggctacaccaactttgctttcgttcaccaagcccatgtctctacttgggtgtctaatgggcatctccaacattacctaccccaaacagaaaaccctttcttccccccaaccacaccccaccctacccccacagtattttctccatgcccggaaagatctgctctcttatggtccctctttgcctcactgaaaagcaggacaagttggggacttcccaaacttttatgcatgaagaaacccaggcaatttgccaaaaggtacactctgggggtctgtcatttactctgagccagaaccctgaaatttttactaacccatcacataatgaatgaagagaatctttttctttttttttttttttctttttttttggtttttcgagacagggtttctctgtatagccctggctatcctggaacacactctgtagaccaggctggcctcgaactcagaaatccacctgcctctgcctcccgagtgctgggattaaaggcgtgcgccaccacgcctggctgaatgaagagaatcttgacctcatctccccagcctcttggtcctgagggaccctggtctacctactgctttgctgtcttcttagctcttcttacttttttgctgactcagacctatggctatctccattatacagatgaggagactgaggcatggatccctggttggtccatggtcacgtgaagcccatcacccagtatttgtaaagtgagatgggccaggctggtaccttggaactgaaactcacactgccctacctggaagaatctgacaggcaaaatctgctgctgaaagtgattgtctgtcacgtttctcagctgcccgactctgagaactccacagccccctttcgttccaccatactacagagtcgccacggaaagccggctctgtggagaagctgaggtagctgggtttctgtctgggttactctgtccagcgaggaaacaagtaccttagacccactaagcctctgctttctgaactgtaaagtgggggatatgacacctgcctcccagggatggctgaatgctctggcagaagcttagagcccccacagctacccctaggctcacagctcctccgatgagacctagaattgaggtatgagttgaataccccaggcaggtccaaggcttccacgggcccaggctgaccaagctgaggccgcccaccgtagggcttgcctatctgcaggcagctcacaaaggaacaataacaggaaaccatcccgaggggaagtgggccagggccagttggaaaacctgcctccctcccagcctgggtgtggctcccctctcccctcctgaggcaatcaactgtgctctccacaaagctcggccctggacagactgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgagctggagcctcggtagccgttcctcctgcccgctgggcctcccaacgggccctcctcccctccttgcaccggcccttcctggtctttgaataaacattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttc

SEQ ID NO: 17 - DNA, 3299 bp, synthetic construct
  misc_feature 1..3299: note = Made in Lab - full polynucleotide sequence of vector genome
  source 1..3299: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacatacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 18 - skipped

SEQ ID NO: 19 - DNA, 3750 bp, synthetic construct
  misc_feature 1..3750: note = Made in Lab - full polynucleotide sequence of vector genome
  source 1..3750: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 20 - skipped

SEQ ID NO: 21 - DNA, 3745 bp, synthetic construct
  misc_feature 1..3745: note = Made in Lab - full polynucleotide sequence of vector genome
  source 1..3745: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacatatttgcttctaggaagcagaagactgaggaaatgacttgggcgggtgcatcaatgcggccaaaaaagacacggacacgctcccctgggacctgagctggttcgcagtcttcccaaaggtgccaagcaagcgtcagttcccctcaggcgctccaggttcagtgccttgtgccgagggtctccggtgccttcctagacttctcgggacagtctgaaggggtcaggagcggcgggacagcgcgggaagagcaggcaaggggagacagccggactgcgcctcagtcctccgtgccaagaacaccgtcgcggaggcgcggccagcttcccttggatcggactttccgcccctagggccaggcggcggagcttcagccttgtcccttccccagtttcgggcggcccccagagctgagtaagccgggtggagggagtctgcaaggatttcctgagcgcgatgggcaggaggaggggcaagggcaagagggcgcggagcaaagaccctgaacctgccggggccgcgctcccgggcccgcgtcgccagcacctccccacgcgcgctcggccccgggccacccgccctcgtcggcccccgcccctctccgtagccgcagggaagcgagcctgggaggaagaagagggtaggtggggaggcggatgaggggtgggggaccccttgacgtcaccagaaggaggtgccggggtaggaagtgggctggggaaaggttataaatcgcccccgccctcggctgctcttcatcgaggtccgcgggaggctcggagcgcgccaggcggacactcctctcggctcctccccggcagcggcggcggctcggagcgggctccggggctcgggtgcagcggccagcgggcgcctggcggcgaggattacccggggaagtggttgtctcctggctggagccgcgagacgggcgctcagggcgcggggccggcggcggcgaacaagaggacggactctggcggccgggtcgttggccgcggggagcgcgggcaccgggcgagcaggccgcgtcgcgctcaccgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 22 - skipped

SEQ ID NO: 23 - DNA, 4316 bp, synthetic construct
  misc_feature 1..4316: note = Made in Lab - full polynucleotide sequence of vector genome
  source 1..4316: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataagctcctcccagcctcaggcccaggaatgggaatctctgtgggtcacacatcagtagggaggtctttcccgatccttttctatgctactccaggagtcaaagcgtctcctgggacttttcagggcgcttcagaagagccctgggcctaaaccagctcaaccaagctgcagggacccagcctcctgagaaaagtgaatgtgagcccggtgcattcagaggagaatgaagccttcacccagaacacactctgggaagatgtcccaggcccagggggagggtttgtactaccagacctaagtcacctaaactgacaccaagtctcatccatcccaaccattccattccgggtcagaggggtcatcgatttaaccagcaaggctgcccatccaacggttgctccctctgctccctggaagggcctcctcgtgggcgttctgtacctacaggtcttgttccgttctgggaactgccagtggtggcaagaggtggagcaacgggtgccagggcagggagaggtgagtctgggagggaagcagaggcaagatccatggggctttagagactttgccaaagcagtgcgactgctcccaggttgttgtcagccgtcaagagtgagtgcacctccctgggcagacttctgctgccccagtgcccaggaataggcaggggtttgccgcaaaatgaatgacacctggcagacaataagctgaagctttcattagcagcttaagctgaggactatctatgcaaccgatactccctgtgtgctccccgggactgcttaatgtgagcccttgtggagcgattggcaccaagaaagcaaggactaagtcagaagttcaagtcccagccttgccacagcctcagggtgccctcgagcacagcaagcctcagttttcccatctgtacaatgagagaggtacacaaggtagactcgaaggctctttgttgccagggccctgtgttcctttgagtgtatgtgcttctcaggcccacagaggtcctttgtgtttcgtatgtgaactgctctctaggaaacccatgtaactgtctgtgtcctggggcacatacatgaggactcatgtgggccgtattgtgtgtttgtgccggggggaggggagaccccagaacaatgtcccccaccccacccccctcctcaataggcggaagccactggcttcctccctttcctgcctcctgcctcctttgtgccagcaagactgagtactggagagagacaggggatgggaaaaatcagtccagctgtccccaggtctgcccttaccataaccttccccccacctcaagtgactcctcccaggccacacccatccccagccttgtgggggccagattggggggcctagaggctcaaaggcagaatgagtcctcccaccccctaccctgccacccctcccacccaagccacctcatttcctcttcctccccagcaccgacccacactgaccaacacaggctgagcagtcaggcccacagcatctgaccccaggcccagctcgtcctggctggcctgggtcggcctctggagtatggtctggcgggtgccccctttcttgctccccatcctcttcttggcttctcatgtgggccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 24 - skipped

SEQ ID NO: 25 - DNA, 4716 bp, synthetic construct
  misc_feature 1..4716: note = Made in Lab - full polynucleotide sequence of vector genome
  source 1..4716: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacatactagtagcagaaacaaggtcctctggaagagcaactgatgctcttaggtactgaagcatcatcctgccccagagaccactcgcatatgaagcacacatattcagtctgccttacttgtgttaatgattgccagtgtccctctgacctcctagccctgaaaagtgtggcctgaaggtcatttcagagacggggagagctgctcagagaagccaatcggcgagtctaggacacacagacaggatctagtcccagagttcgctagcctaggtgagcgtcccctggccccttataccacttccttctccagcttgcatctaatctgctctggcagaccatcgtgtttcctgtcttcctggcagcctccagcacgctcagtgctactccctgcgcatgcgccctcctcccagtaccttctctgactccagtgggcttggagtgcgaggaggaagggtgaggaaggggtgaaatcaggtattggatccacagggggtctgaagagcactagcctggccttttgggactgaacttctgctatgaagacctccactgccatccctggagtccggggcacatccaaggcttgctgtccatcgtttactgtttacagatgacaacaatgactgtgttcggggcagaaatatccaccagggctagagtacaaaaggagtttgcattgatggccggacaggccctgtccctggcagcctgccagcgctgagtatgagacccagcgggaagtgctaccctggcagacgtgtccactgagtacacagaccaccaaggcaggcagctctcggggaagctgtctatgctgggccagcccaccttgagggcagggaacagaacagattgtggcagagaggaaaatgtggagcttctgtttgttcacagacacacgcactcgcccacgcacgcacgcacgcacgcacgcacgcacgaatgcacgcacgcagtagttgaatgctatggattccgctcagagctgagaacagccccagcgacagttccctggcctctctccttactctgatgtcctcatctgtcttcacatggtctcaggacgctaatactccatcctaatgtacactcctttccctgggcctccgttccagttcagttctcagaggacctggagggagtgattggctacaccaactttgctttcgttcaccaagcccatgtctctacttgggtgtctaatgggcatctccaacattacctaccccaaacagaaaaccctttcttccccccaaccacaccccaccctacccccacagtattttctccatgcccggaaagatctgctctcttatggtccctctttgcctcactgaaaagcaggacaagttggggacttcccaaacttttatgcatgaagaaacccaggcaatttgccaaaaggtacactctgggggtctgtcatttactctgagccagaaccctgaaatttttactaacccatcacataatgaatgaagagaatctttttctttttttttttttttctttttttttggtttttcgagacagggtttctctgtatagccctggctatcctggaacacactctgtagaccaggctggcctcgaactcagaaatccacctgcctctgcctcccgagtgctgggattaaaggcgtgcgccaccacgcctggctgaatgaagagaatcttgacctcatctccccagcctcttggtcctgagggaccctggtctacctactgctttgctgtcttcttagctcttcttacttttttgctgactcagacctatggctatctccattatacagatgaggagactgaggcatggatccctggttggtccatggtcacgtgaagcccatcacccagtatttgtaaagtgagatgggccaggctggtaccttggaactgaaactcacactgccctacctggaagaatctgacaggcaaaatctgctgctgaaagtgattgtctgtcacgtttctcagctgcccgactctgagaactccacagccccctttcgttccaccatactacagagtcgccacggaaagccggctctgtggagaagctgaggtagctgggtttctgtctgggttactctgtccagcgaggaaacaagtaccttagacccactaagcctctgctttctgaactgtaaagtgggggatatgacacctgcctcccagggatggctgaatgctctggcagaagcttagagcccccacagctacccctaggctcacagctcctccgatgagacctagaattgaggtatgagttgaataccccaggcaggtccaaggcttccacgggcccaggctgaccaagctgaggccgcccaccgtagggcttgcctatctgcaggcagctcacaaaggaacaataacaggaaaccatcccgaggggaagtgggccagggccagttggaaaacctgcctccctcccagcctgggtgtggctcccctctcccctcctgaggcaatcaactgtgctctccacaaagctcggccctggacagactgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgagctggagcctcggtagccgttcctcctgcccgctgggcctcccaacgggccctcctcccctccttgcaccggcccttcctggtctttgaataaacattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 26 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 27 - DNA, 1476 bp, synthetic construct
  misc_feature 1..1476: note = Made in Lab - Codon-optimized polynucleotide encoding GLUT1
  source 1..1476: mol_type = other DNA
atggaaccatcatccaaaaagctgaccggacgactgatgcttgcagttggcggtgcggtcttggggagcctgcagtttgggtacaatactggcgtaatcaatgccccgcagaaggttattgaagaattttacaatcaaacgtgggtacatcgctacggtgaatccattcttcctacaactctgaccacactctggagcctttctgtagcgattttttccgtcgggggcatgataggatcattttccgtcggtctttttgtgaaccgctttggccggagaaattccatgctgatgatgaatcttctcgctttcgtgagtgccgtcctcatgggatttagtaaactgggtaaatctttcgagatgttgatactggggagatttattatcggcgtgtattgtggtttgaccacgggctttgtaccaatgtatgttggcgaggtttctccgacagcattgagaggtgcactcgggaccttgcaccagttgggcatcgtagtaggaatccttatagcgcaagttttcgggctcgattccatcatggggaacaaagatctctggccattgctcctctcaataatttttataccggcattgcttcagtgtattgttcttcctttttgcccagagtcccctaggttcctgctcataaacaggaatgaggagaatcgcgctaagtccgtgttgaaaaaacttaggggaactgcagacgttactcacgatttgcaagagatgaaggaggaatctaggcaaatgatgcgcgagaagaaggttaccatactcgaactcttccgctcccccgcgtacaggcagcccattcttatcgcggtcgtcttgcagttgtcacaacagttgagtgggattaatgcagttttctattatagcacgtccatatttgaaaaagcaggcgtccaacaacctgtctatgcaactataggctcaggcattgtaaacacagcgtttactgtagtatcactgtttgtcgttgagcgggctggtcgaaggaccttgcacctcataggactggcgggcatggcgggctgtgcgattcttatgacaattgcgctcgcgctgttggaacagcttccgtggatgtcctatctctctatagtagcaatatttggatttgttgcattttttgaagttgggcccggacctatcccctggttcatcgtcgcggagctcttttcccaaggcccaagaccggctgccattgctgttgcaggcttctcaaactggacgagtaatttcatagtaggtatgtgtttccagtatgttgaacagctctgtgggccctatgtctttatcatctttactgtgttgctcgtgttgttctttatcttcacttatttcaaagtacccgagacaaagggcaggacgtttgacgagattgcatctggttttagacaaggaggtgcctcacagagtgataaaaccccggaggaattgtttcatccgctgggagccgactcacaggtc

SEQ ID NO: 28 - DNA, 10 bp, synthetic construct
  misc_feature 1..10: note = Kozak sequence motif
  source 1..10: mol_type = other DNA
gccaccatgg

SEQ ID NO: 29 - DNA, 1482 bp, synthetic construct
  misc_feature 1..1482: note = Polynucleotide encoding GLUT1 with Kozak motif
  source 1..1482: mol_type = other DNA
gccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtg

SEQ ID NO: 30 - RNA, 13 nt, synthetic construct
  misc_feature 1..13: note = Kozak sequence motif
  source 1..13: mol_type = other RNA
gccgccrccatgg

SEQ ID NO: 31 - RNA, 10 nt, synthetic construct
  misc_feature 1..10: note = Kozak sequence motif
  source 1..10: mol_type = other RNA
gacaccatgg

SEQ ID NO: 32 - DNA, 141 bp, unidentified
  source 1..141: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 33 - DNA, 170 bp, unidentified
  source 1..170: mol_type = genomic DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgta

SEQ ID NO: 34 - DNA, 141 bp, unidentified
  source 1..141: mol_type = genomic DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 35 - DNA, 124 bp, synthetic construct
  misc_feature 1..124: note = Made in Lab - vector filler sequence
  source 1..124: mol_type = other DNA
gcggcaattcagtcgataactataacggtcctaaggtagcgatttaaatacgcgctctcttaaggtagccccgggacgcgtcaattgactacaaaccgagtatctgcagagggccctgcgtatg

SEQ ID NO: 36 - DNA, 84 bp, synthetic construct
  misc_feature 1..84: note = Made in Lab - vector filler sequence
  source 1..84: mol_type = other DNA
cttctgaggcggaaagaaccagatcctctcttaaggtagcatcgagatttaaattagggataacagggtaatggcgcgggccgc

SEQ ID NO: 37 - DNA, 63 bp, synthetic construct
  misc_feature 1..63: note = Made in Lab - vector filler sequence
  source 1..63: mol_type = other DNA
gttacccaggctggagtgcagtggcacatttctgctcactgcaacctcctcctccctgggttc

SEQ ID NO: 38 - DNA, 573 bp, synthetic construct
  misc_feature 1..573: note = Made in lab - CAG promoter in part Human betaherpesvirus 5
  source 1..573: mol_type = other DNA
acttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 39 - DNA, 253 bp, Homo sapiens
  source 1..253: mol_type = genomic DNA
gcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagtc

SEQ ID NO: 40 - DNA, 281 bp, Gallus gallus
  source 1..281: mol_type = genomic DNA
ggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggga

SEQ ID NO: 41 - DNA, 220 bp, Human betaherpesvirus 5
  source 1..220: mol_type = genomic DNA
tggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccg

SEQ ID NO: 42 - DNA, 583 bp, Human betaherpesvirus 5
  source 1..583: mol_type = genomic DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgt

SEQ ID NO: 43 - DNA, 508 bp, Human betaherpesvirus 5
  source 1..508: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 44 - DNA, 573 bp, synthetic construct; identical to SEQ ID NO: 38
  misc_feature 1..573: note = Made in lab - CAG promoter in part Human betaherpesvirus 5
  source 1..573: mol_type = other DNA

SEQ ID NO: 45 - DNA, 580 bp, synthetic construct
  misc_feature 1..580: note = Made in lab - CAG promoter in part Human betaherpesvirus 5
  source 1..580: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 46 - DNA, 455 bp, Homo sapiens
  source 1..455: mol_type = genomic DNA
caacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaatagcatgcgtgaggctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgtt

SEQ ID NO: 47 - DNA, 401 bp, Homo sapiens
  source 1..401: mol_type = genomic DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 48 - DNA, 448 bp, Homo sapiens
  source 1..448: mol_type = genomic DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 49 - DNA, 422 bp, Homo sapiens
  source 1..422: mol_type = genomic DNA
ctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctc

SEQ ID NO: 50 - DNA, 281 bp, Homo sapiens
  source 1..281: mol_type = genomic DNA
acttgtggacaaagtttgctctattccacctcctccaggccctccttgggtccatcaccccaggggtgctgggtccatcccacccccaggcccacacaggcttgcagtattgtgtgcggtatggtcagggcgtccgagagcaggtttcgcagtggaaggcaggcaggtgttggggaggcagttaccggggcaacgggaacagggcgttttggaggtggttgccatggggacctggatgctgacgaaggctcgcgaggctgtgagcagccacagtgccctgc

SEQ ID NO: 51 - DNA, 851 bp, synthetic construct
  misc_feature 1..851: note = Made in Lab - eSYN promoter polynucleotide
  source 1..851: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggctgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtcgcgactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagg

SEQ ID NO: 52 - DNA, 304 bp, Human betaherpesvirus 5
  source 1..304: mol_type = genomic DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 53 - DNA, 953 bp, Homo sapiens
  source 1..953: mol_type = genomic DNA
cgcgtccgcccgcgagcacagagcctcgcctttgccgatccgccgcccgtccacacccgccgccaggtaagcccggccagccgaccggggcatgcggccgcggcccttcgcccgtgcagagccgccgtctgggccgcagcggggggcgcatggggcggaaccggaccgccgtggggggcgcgggagaagcccctgggcctccggagatgggggacaccccacgccagttcgcaggcgcgaggccgcgctcgggcgggcgcgctccgggggtgccgctctcggggcgggggcaaccggcggggtctttgtctgagccgggctcttgccaatggggatcgcacggtgggcgcggcgtagcccccgtcaggcccggtgggggctggggcgccatgcgcgtgcgcgctggtcctttgggcgctaactgcgtgcgcgctgggaattggcgctaattgcgcgtgcgcgctgggactcaatggcgctaatcgcgcgtgcgttctggggcccgggcgcttgcgccacttcctgcccgagccgctggcgcccgagggtgtggccgctgcgtgcgcgcgcgcgacccggtcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcggcagcggctgctcttggcggccccgaggtgactatagccttcttttgtgtcttgatagttcgccagcctctgctaaccatgttcatgccttcttctttttcctacagctcctgggcaacgtgctggttattgtgctgtctcatcattttggcaaagaattc

SEQ ID NO: 54 - DNA, 1068 bp, synthetic construct
  misc_feature 1..1068: note = Made in Lab - Chicken beta-actin exon/intron plus rabbit globin intron
  source 1..1068: mol_type = other DNA
gtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 55 - DNA, 126 bp, Homo sapiens
  source 1..126: mol_type = genomic DNA
agtctgcggtgggcagcggaggagtcgtgtcgtgcctgagagcgcagctgtgctcctgggcaccgcgcagtccgcccccgcggctcctggccagaccacccctaggaccccctgccccaagtcgca

SEQ ID NO: 56 - DNA, 121 bp, Human betaherpesvirus 5
  source 1..121: mol_type = genomic DNA
tcagatcgcctggagaggccatccacgctgttttgacctccatagtggacaccgggaccgatccagcctccgcggccgggaacggtgcattggaacgcggattccccgtgccaagagtgac

SEQ ID NO: 57 - DNA, 512 bp, synthetic construct
  misc_feature 1..512: note = Made in Lab - adenovirus derived enhancer element
  source 1..512: mol_type = other DNA
ctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccag

SEQ ID NO: 58 - DNA, 956 bp, Homo sapiens
  source 1..956: mol_type = genomic DNA
ctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacgtgctgcgacgctttttttctggcaagatagtcttgtaaatgcgggccaggatctgcacactggtatttcggtttttgggcccgcggccggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttggcctttttgagtttggatcttggttcattctcaagcctcagacagtggttcaaagtttttttcttccatttcag

SEQ ID NO: 59 - DNA, 939 bp, Homo sapiens
  source 1..939: mol_type = genomic DNA
gtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcag

SEQ ID NO: 60 - DNA, 83 bp, Homo sapiens
  source 1..83: mol_type = genomic DNA
tcagaagccccgggctcgtcagtcaaaccggttctctgtttgcactcggcagcacgggcaggcaagtggtccctaggttcggg

SEQ ID NO: 61 - DNA, 476 bp, Homo sapiens
  source 1..476: mol_type = genomic DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 62 - DNA, 589 bp, synthetic construct
  misc_feature 1..589: note = Made in Lab - mutated woodchuck hepatitis regulatory element
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 63 - DNA, 588 bp, synthetic construct
  misc_feature 1..588: note = Made in Lab - mutated woodchuck hepatitis regulatory element
  source 1..588: mol_type = other DNA
tcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 64 - DNA, 755 bp, synthetic construct
  misc_feature 1..755: note = Made in Lab - mutated woodchuck hepatitis regulatory element
  source 1..755: mol_type = other DNA
ttcctgttaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 65 - DNA, 12 bp, synthetic construct
  misc_feature 1..12: note = CAAX motif
  source 1..12: mol_type = other DNA
tgtgtgataatg

SEQ ID NO: 66 - DNA, 810 bp, Homo sapiens
  source 1..810: mol_type = genomic DNA
ctgttctcatcacatcatatcaaggttatataccatcaatattgccacagatgttacttagccttttaatatttctctaatttagtgtatatgcaatgatagttctctgatttctgagattgagtttctcatgtgtaatgattatttagagtttctctttcatctgttcaaatttttgtctagttttattttttactgatttgtaagacttctttttataatctgcatattacaattctctttactggggtgttgcaaatattttctgtcattctatggcctgacttttcttaatggttttttaattttaaaaataagtcttaatattcatgcaatctaattaacaatcttttctttgtggttaggactttgagtcataagaaatttttctctacactgaagtcatgatggcatgcttctatattattttctaaaagatttaaagttttgccttctccatttagacttataattcactggaatttttttgtgtgtatggtatgacatatgggttcccttttattttttacatataaatatatttccctgtttttctaaaaaagaaaaagatcatcattttcccattgtaaaatgccatatttttttcataggtcacttacatatatcaatgggtctgtttctgagctctactctattttatcagcctcactgtctatccccacacatctcatgctttgctctaaatcttgatatttagtggaacattctttcccattttgttctacaagaatatttttgttattgtctttgggctttctatatacattttgaaatgaggttgacaagtta

SEQ ID NO: 67 - DNA, 726 bp, Hepatitis B virus
  source 1..726: mol_type = genomic DNA
ataacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggagcaaacctcatcgggaccgacaattctgtcgtactctcccgcaagtatacatcgtttccatggctgctaggctgtgctgccaactggatcctgcgcgggacgtcctttgtttacgtcccgtcggcgctgaatcccgcggacgacccctcccggggccgcttggggctctaccgcccgcttctccgtctgccgtaccgtccgaccacggggcgcacctctctttacgcggactccccgtctgtgccttctcatctgccggaccgtgtgcacttcgcttcacctctgcacgtcgcatggaggccaccgtgaacgcccaccggaacctgcccaaggtcttgcataagaggactcttggactttcagcaatgtcatc

SEQ ID NO: 68 - DNA, 755 bp, synthetic construct
  misc_feature 1..755: note = Made in Lab - HepB derived enhancer element
  source 1..755: mol_type = other DNA
ttcctgtaaacaggcctattgattggaaagtttgtcaacgaattgtgggtcttttggggtttgctgccccttttacgcaatgtggatatcctgctttaatgcctttatatgcatgtatacaagcaaaacaggcttttactttctcgccaacttacaaggcctttctcagtaaacagtatatgaccctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagctggactggagcaaacctcatcgggaccgacaattctgtcgtactctcccgcaagcactcaccgtttccgcggctgctcgcctgtgttgccacctggattctgcgcgggacgtccttctgctacgtcccttcggccctcaatccagcggaccttccttcccgcggcctgctgccggctctgcggcctcttccgcctcttcgccttcgccctcagacgagtcggatctccctttgggccgcctccccgcccatgtatctttttcacctgtgccttgtttttgcctgtgttccgcgtcctacttttcaagcctccaagctgtgccttgggcggctttggggcatggacatagatccctataaagaatttggttcatcttatcagttgttgaattttcttcctttggac

SEQ ID NO: 69 - DNA, 94 bp, Homo sapiens; identical to SEQ ID NO: 4
  source 1..94: mol_type = genomic DNA

SEQ ID NO: 70 - DNA, 596 bp, Woodchuck hepatitis virus
  source 1..596: mol_type = genomic DNA
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 71 - DNA, 387 bp, Oryctolagus cuniculus
  source 1..387: mol_type = genomic DNA
tggctaataaaggaaatttattttcattgcaatagtgtgttggaattttttgtgtctctcactcggaagaacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 72 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaatacaatagcaggcatgctggggatgcggtgggctctatgggtacccaggtgctgaagaattgacccggttcctcctggg

SEQ ID NO: 73 - DNA, 251 bp, Bos taurus
  source 1..251: mol_type = genomic DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctggg

SEQ ID NO: 74 - DNA, 225 bp, Bos taurus
  source 1..225: mol_type = genomic DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 75 - DNA, 202 bp, Homo sapiens
  source 1..202: mol_type = genomic DNA
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 76 - protein, 735 aa, unidentified [sequence omitted]

SEQ ID NO: 77 - protein, 736 aa, unidentified
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 78 - protein, 736 aa, unidentified
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 79 - protein, 738 aa, unidentified
  REGION 1..738: note = Non-human primate adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 80 - protein, 738 aa, unidentified
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 81 - protein, 738 aa, unidentified
  REGION 1..738: note = Non-human primate adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 82 - protein, 743 aa, synthetic construct
  REGION 1..743: note = Synthetic construct - AAV9 variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 83 - protein, 7 aa, synthetic construct
  REGION 1..7: note = Peptide insert
TLAVPFK

SEQ ID NO: 84 - protein, 7 aa, synthetic construct
  REGION 1..7: note = Peptide insert
KFPVALT

SEQ ID NO: 85 - DNA, 940 bp, Homo sapiens
  source 1..940: mol_type = genomic DNA
tggagccgccaaatattttgggaaatagcgggaatgttggcgaactgggcaagtgcgttttctgattaagagcaaccagattcagctttttaaactacaattatactggccaaacaaaatacccttatacaaaaaccaaaactactggcaggagtcgctgccagcttgcgacccggcatacttggctgagtatccgcttctcccttgtggctccaaactgctgcagattctcggccacttcagacgcgcgcgatggcgaagagggtcctgcactttgacgcgcctggtgagggagcgctgctcttcgcagcgctcctggtgatgctccccaaatttcggggaccggcaagcgattaaatcttggagttgctcagcgcccgttaccgagtactttttatttacaccagaaacaaagttgttgctctgggatgttctctcctgggcgacttggggcccagcgcagtccagttgtgtggggaaatggggagatgtaaatgggcttggggagctggagatcgccgccgggtacccgggtgaggggcggggctggccgcacgggagagcccctcctccgctccggccccgccccgcatggccccgcctccgcgctctagagtttcggcaccagctcccaccctgcactgagtcccgggaccccgggagagcggtcaatgtgtggtcgctgcgtttcctctgcctgcgccgggcatcacttgcgcgccgcagaaagtccgtctggcagcctggatatcctctcctaccggcacccgcagacgcccctgcagccgcggtcggcgcccgggctccctagccctgtgcgctcaactgtcctgcgctgcggggtgccgcgagttccacctccgcgcctccttctctagacaggcgctgggagaaagaaccggctcccgagttctgggcatttcgcccggctcgaggtgca

SEQ ID NO: 86 - DNA, 1142 bp, Mus musculus
  source 1..1142: mol_type = genomic DNA
aagcttccgaccgttagtcagagaactgtaagtgctcagagcctggctgacaatgatctggaatgaaccagataacaacataataaaatctcagtaaaataatttaacagttagcttggaagctggtcagctctggggaaatcagggtaaattgtgctgtcatgaactgtcccacactgacatcggccaaagtgaatatgaactttggtagatccaatgcctgttctatttatttttccagtgaaaagtattttgatagagcttttcattttgtaaatacactgagttaaccaaaatatcatggatttccgtttgttcttaagacatgcaactcgtctacggctataccactctgaacgcgcccgatctcggaagacatgcaactcaaatgtaaatacagtagaatattacttaggtagaaactcctggtgattttaaaagattggaaaagaatatgaggaagagttgaataatgcaaattctagtgtgtgtgctaccgaagtgaacacttaatgcacagtctacagactaggacattttatcgtgtgttgtaaaattgggtagaaacttgtgtttgtgaaaactgagcattaaaaccttacagagaccgtttcttgtttacttttgaaaaaaaaaagagtcacgtgagcctcattttgtatttgtgtgtgtgtgtgtgtgtgtgtctcccctcctcccagcgtgtgtgtgctgggaggaggggagaccccagaacaatgtcctgcctccaaaccttctcaataggcggaagccactggcttcctccctttcctgtctcccgtgctccagcaatgcagatggaagggaccgaagggatgggagagagagcccaaccatccccagatctgtccttgtcacaacctgcctcccacctctaatgcccccccttccagagacttccaggccacacccatcccgggcttgtgggggctggacacgggaggactacaggcgacaactcttcccaccctctctccctgccacccctcctaccctaaccatcatttcctcttcctccccagcaccgaggtgcactgagctggacaggctgaacactcagacccacagcaactgaccccgggcccagctggccttggctggcccagggcagcttccagagt

SEQ ID NO: 87 - DNA, 2079 bp, Homo sapiens
  source 1..2079: mol_type = genomic DNA
gctggagtgcagtggcacgatctcggctcactgcaacctctgcctcccaggttcaaacaattctcctgcctcagcctccagagtagctggggttacaggtgcacgccagcaagcacagctaaattttgtatttttagtagagatggggttttgccatgttggccaggctggtctcaaactcctgacctcaggtgatccactcccaaagtgctgggattataggcgtgagccactgtgccaggcccactgtttttgtttttttttttcgtgatgacaaatttaaagtcatctcataggaatagaaaatagctttttagtagaagctcttggaatttaaattgagactgaatggaaagatgaaagaaaataaacttattaacatttaatgagaaccttcaaagaactaggcatagtaccaaatggttttatatttttaaacctcatttattcctctcaaaacacctgggaaggagatatttttgccatttcacagctgttgaaactgaggctcaaaaagactaagtaacttttctcagctacacatgtggctgagccagtatttgaacccagttctgtttgcagacagaacctgggctttttcacacctgcaaactggaaacattaattggttcttaagatcatcatcgatgtgataaaacctgggacagaaattagtcaagactagctgcatctgccttttcctctggtgggtaggaaaaggaggagtataatgatttcctcaggcatgaaggtcgatgatgagcaaagtgtatactctctaatctaatgtcataattcatattgtggagtaattatctggataagtgtagggtctctgacctcattctagatattgtacattccatggctattttcattttggtccatgaactctctttgctctcatgagcaccatttttatcccaatctaatcctgtatgtttgtgtttttacacagattagtttttaaatgttatatataatttgcttctgaaacaccattgctcaatgactaccaaatctttctcattaccaaaatccttctatgccaacttcttcaagaaatttgatcacctttagatgaattgttaatgaaaattaaagctatagccggcaacatgggtatctttgggctaatggccaaccaacaggccatctgtgtgaaagaaaacaggctaacaattttggactctggtctcttggggctacattgagcattgacctcaccggtgctcactgaaattaattgcttttcaggttgtattttctcatcacggaaaccttcttctcccaattcaaaccatgtgggttaaaatgagaaaacaaaagccaaaacggcttcccacacccaaaagctccttctgtcagagatcccagtagccccgggagagctgttagaagtctgagaaggattggtcatcatcgcataccatacataggtggagggcttgttattctcagtttcccgcctatgagaggatacccctattgtttctgaaaatgctgaccgggacccacacttccaacaaaaattcctctgcccctacagcagcagcaaaagcagcagcagaagcaacagcaacagataagtgttttgatgaattgcgagatggatagggcttgagtgcccccagccctgctgataccaaatgcctttaagatacagcctttcccatcctaatctacaaaggaaacaggaaaaaggaacttaaaactccctgtgctcagacagaaatgagactgttacagcctgcttctgtgctgttccttcttgcctctaacttgtaaacaagacgtagtaggacgatgctaatggaaagtcacaaaccgctgggtttttgaaaggatccttgggacctcatgcacatttgtggaaactggatggagagatttggggaagcatggactctttagccagcttagttctctgtggagtcagcttgctcctttctggtaaggtttggctttattttttttaatttagtattttaaaaaacagagttagtgatttctgggtgctctccccaaatctcatcagtgctgatgaacaaggggtggctgtagcaaaggcaccatttc

SEQ ID NO: 88 - DNA, 1559 bp, Homo sapiens
  source 1..1559: mol_type = genomic DNA
catccatgcccatggcctcagatgccagccataagctgttgggttccaaacctcgactccaggctggactcacccctgtctcccccaccagcctgacacctccacctgggtatctaacgagcatctcaaactcaacctgcctgagacagaggaatcactatcccctcctcctccaaaaatatccttccatcacactccccatcttgtgctctgatttactaaacggccctgggccctctctttctcagggtctctgcttgcccagctatataataaaacaagtttgggacttcccaaccattcacccatggaaaaacagaagcaactcttcaaaggacagattcccaggatctgccctgggagattccaaatcagttgatctggggtgagcccagtcctctgtagtttttagaagctcctcctatgtctctcctggtcagcagaatcttggcccctcccttccccccagcctcttggttcttctgggctctgatccagcctcagcgtcactgtcttccacgcccctctttgattctcgtttatgtcaaaagccttgtgaggatgaggctgtgattatccccattttacagatgaggaaactgtggctccaggatgacacaactggccagaggtcacatcagaagcagagctgggtcacttgactccacccaatatccctaaatgcaaacatcccctacagaccgaggctggcaccttagagctggagtccatgcccgctctgaccaggagaagccaacctggtcctccagagccaagagcttctgtccctttcccatctcctgaagcctccctgtcacctttaaagtccattcccacaaagacatcatgggatcaccacagaaaatcaagctctggggctaggctgaccccagctagatttttggctcttttataccccagctgggtggacaagcaccttaaacccgctgagcctcagcttcccgggctataaaatgggggtgatgacacctgcctgtagcattccaaggagggttaaatgtgatgctgcagccaagggtccccacagccaggctctttgcaggtgctgggttcagagtcccagagctgaggccgggagtaggggttcaagtggggtgccccaggcagggtccagtgccagccctctgtggagacagccatccggggccgaggcagccgcccaccgcagggcctgcctatctgcagccagcccagccctcacaaaggaacaataacaggaaaccatcccagggggaagtgggccagggccagctggaaaacctgaaggggaggcagccaggcctccctcgccagcggggtgtggctcccctccaaagacggtcggctgacaggctccacagagctccactcacgctcagccctggacggacaggcagtccaacggaacagaaacatccctcagcccacaggcacggtgagtgggggctcccacactcccctccaccccaaacccgccaccctgcgcccaagatgggagggtcctcagcttccccatctgtagaatgggcatcgtcccactcccatgacagagaggctcc

SEQ ID NO: 89 - DNA, 399 bp, Homo sapiens
  source 1..399: mol_type = genomic DNA
gtctcccaggcatgactccaacaatgcatcccatgggatttggggttccccagatctggggcttgtaggcctgactctcccctgtgcacacgtctcatacacgcatgcgtgcacccattgcctgccccgccccttgcacagggagtcagcagggaggactgggttatgccctgcttatcagcagcttcccagcttcctctgcctggattcttagaggcctggggtcctagaacgagctggtgcacgtggcttcccaaagatctctcagataatgagaggaaatgcagtcatcagtttgcagaaggctagggattctgggccatagctcagacctgcgcccaccatctccctccaggcagcccttggctggtccctgcgagcccgtggagactgccagtc

SEQ ID NO: 90 - DNA, 735 bp, Homo sapiens
  source 1..735: mol_type = genomic DNA
atctttagccgatccattcaaccctggccaggatccaaatggactgtttttgtcagggccaggaccggatccttcatacctggggtgcataggaagtgttagtactccccttcctccaaacacagcagcaaaattggctcaggttgaggtgtttttctcaacttccctggagtccagccctggaagctggatcaggaagctgtgttgttctactgtgattccccctggcctgtatcagcttgccctgaaacaaccagcattcctggttatcccacacaggtggggcactctaggaagaccagggatcaagtgtgggggtgtagggatagggggtgtttggggagggcaaggcagttaattaaggcagctgccaggaggtctccctccaaactctacaaagctttatcagcttggaggtacttctaataccatttcctttcattgtttccttttggtaattaaaaggaggccaatcccctgttgtggcagctcacagctattgtggtgggaaagggagggtggttggtggatgtcacagcttgggctttatctcccccagcagtggggactccacagcccctgggctacataacagcaagacagtccggagctgtagcagacctgattgagcctttgcagcagctgagagcatggcctagggtgggcggcaccattgtccagcagctgagtttcccagggaccttggagatagccgcagccctcatttgcagggga

SEQ ID NO: 91 - DNA, 1132 bp, Homo sapiens
  source 1..1132: mol_type = genomic DNA
tggcttccggagggtggcctgggggctggggtgccagggacaccatcgccactggtgggagggcagggcacagcccctccgtgtccctttgtctctcctgtctgaaggccagagcaggctgctaggcctggggccaccactgcccctgggtgctacacccagtgtgctgggtcactgggaacttcctgaagtggtgtcacctgaactgggcccccaaggatggggtgcgggcagtaccgcaggaagaggagcagcccctgtgaagattgagaggtctgggaagcccctgcggcttgggagagtgggggtcgccaggcagggggaaagcccctgtgccaccgctttttgccagagactcaggctccagagaggcagtgagtggcatggggggtgaggctggggccctgggcctgacctccacacgcctgcctggcctctctgtttgccatgggatgagagagacagtgctgggactcagagcggggctggagagtgagagtgcgagaaagggcctgggtggggcttggaccccggggcgggctttctggagagcccccctacgagggcctctacggcggtgacggggtggggggcttctgcaaaccttggtcagggaagtggagctggctcgagtggaagagaccacccggctcagtcggggatgtgggagtggactgggtggtgcagactgggggtcgagcgccttctgaagtgacggggccgggacgcgcagggaggcggcccaagaagcgcgccctaggccagcccagaatgcgctcggccgcgactaggacaacggcgggtggggctgggggcggctgccgggcggggagcggtcccgcgccctcagctacccctcaagagccgttgtttccctaacttcagctgccagaggctctgtgattggctgcggcacgatgacccgcgcacggattggctgcttcgggccggggggccgggcccgggggacagaatccgcccccgaaccttcaaagagggtaccccccggcaggagctggcagacccaggaggtgcgacagacccgcggggcaaacggactggggccaagagccgggagcgcgggcgcaaaggcaccagggcccgcccagggcgccgcgcagcacggccttgg

SEQ ID NO: 92 - DNA, 888 bp, Homo sapiens
  source 1..888: mol_type = genomic DNA
cgccttgctgtgccactttgggacttccctccctagcctgagcttcagttttcctgcctgttaggcagccccatgtcaactgcacttagtaggccgggtttgatgcccgacaagacgtgaagtggtggaggtgggcaggatcccagcgctaccatcttcttgaaccagtgatctcaacacatcggatttctgtttcctcatctgcaaaatgggatcagtgagctcaggtgggtcacaaattctacaggaactactttagccaagcccggccccctgaaagttcccctcggtgggctgttagggtgattgttttcatctgtggggctccctgatgcgtcccacccaccagccttggagagggtgggatgggagggtggggtgcttggggagacaagcctagagcctgggccctcccaccccactgcctccccccatcccagggccccccacccagtgacaaagcccgtggcacttcctctacccggttggcaggcggcctggcccagccccttctctaaggaagcgcatttcctgcctccctgggccggccgggctggatgagccgggagctccctgctgccggtcataccacagccttcatctgcgccctggggccaggactgctgctgtcactgccatccattggagcccagcaccccctccccgcccatccttcggacagcaactccagcccagccccgcgtccctgtgtccacttctcctgacccctcggccgccaccccagaaggctggagcagggacgccgtcgctccggccgcctgctcccctcgggtccccgtgcgagcccacgccggccccggtgcccgcccgcagccctgccactggacacaggataaggcccagcgcacaggcccccacgtggacacc

SEQ ID NO: 93 - DNA, 1658 bp, Homo sapiens
  source 1..1658: mol_type = genomic DNA
gcccaggctggagtgcagtggcacagtcacaactcactgcagcctcaaactcctgggctcaaaacgatccacagtctcctgagtagctgggactacaggagcttgttaccacacccagctccagtttataaattcatctccagtttataaaggaggaaaccgaggtactgagaggttaaaaaaccttcctgcagacacttgtccagcaagtggccactccaggatttggaccaaggtgatgtgtcttcaggctgtgtctctgccactgtgccacgctgctgggtggtaggcagcagtgggtgggtgcctgcagtggtctgtaaagaccacctgagatgtccttcctcctctgttccaccctgtccaggtccaagaagacagtctatgaagagagagcaggtgtgactctctcagtgtgctcctctgtgagaagcaggctgacatcccaaagggaagggcggataacagagacagtgcaagcggaggagatgagggtgcctcaaagccgggaggctgggtgatgcaggagcctgcgtgtcccgaggggggtgctgggcccagtgtgagtacgtgtgactgtgactgagacagtgtgactgctgaaggcagggacacagcagctccctgactgggggcagaaggcgttaactgtgtgaaggctggttgtgggtgggtgggctctgggcctcgaacccgggggctgagggagatagtaaacagcagggtgactgacgggaagatcatgttggtagccctgcgaagatgctgcagggctgtgggggtttgtgtgactttgcagttcaacaaattcaaattcagccaacgctggcagggcctgttgtgccaggcaaccagctaggaggaggagactcggacccagcttgcagctgaagggcgctggctgccgggttctgtgggttcaccttgcggtgtcttcccttgctaacactgagtccttacaatagccccatctccaggttgaggctagatggaggggacagagggaagtgacttgcccaaggtgacccaagctcccgagtgccagggcaggatctgaattcaggctctcagactgcagagcctgagtccctccctgccatgcctgtgccagggtggaaatgtctggtcctggaggggagcgtggactcctggccttggctctggagacatccccctagaccacgtgggctcctaacctgtccatggtcactgtgctgaggggcgggacggtgggtcacccctagttcttttttccccagggccagattcatggactgaagggttgctcggctctcagagaccccctaagcgccccgccctggccccaagccctcccccagctcccgcgtcccccccctcctggcgctgactccgggccagaagaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgc

SEQ ID NO: 94 - DNA, 1455 bp, Homo sapiens
  source 1..1455: mol_type = genomic DNA
acatccaatgcccgctctgcctcatcttctatgggaaacaagaattttagaggtcaggtagcctaacaccatcaattctcaaaagaggaagctgaggccaagagaagtcctgtgaatttcttacagctcatttgtgacagaccaagaattacccactttactgggttgttatttactaagtgacagtgagtctatatctcttttgacaagtgaggtgggggcatggaattcggcatgtggttggtgtaagaactcccctctctcctctttaaccttacttaataagaccctggcacagttgatattttaagagggctactctgttttcccagagggacctaggcacggtaaccctcttagcatgcagaccttgtttcctgaggggtaatgtttcccttccctgtgacttgtttcttgggggctgtgttctgattttcctgctgagccacttgttgccttgggctggctgccgcgcttggcagtttttagtgagggctctgatagatgccaggaggtgaggggaagggctctgggtggactccgtcattggacaagcagacttagtgatggatgagccttcccctgaggaagttttggatcagaagtccaactgataagtttttccagaattgagtaacccagaagcagtgccgaaaggatcttacctctcttgtggctttttgtattgattttaaaagaaattctcagaggcagttccacattgtactggaagcacagctatatccacaataggcttagatatatgtaacatgaattgctttagaaataacatttgaggagaggggtgagaggaaggaagagagggtcttaaaaaatagccctatcaaaatattttctttcttctaagtattgaaaagacacaatataaccctttcttctttcaaatgatctcatagctatttgttgaggggaaataccaaatgtttattattttttttgaagaagcttcttcggtcctgatgattcatgttgatatcattttcctcctgactacagaggctctgagacaaagctacacctcaagtgatatgccagggtcagaacaattcccgtcctgaaggagggtgtgcaaccttctttatccctccttcacagacgtccttgagcccttgagacggatgtgagtgagtttttcagtcctcatgcaaaacaaccatctaaacataacagatgacatcagcttgggcttttcaattcctggatggcagcagcgtgttaatccagccttcatcctggatttcataaaccaaaacaagagagcctggcaggaggacagcgctgctgctgggttgaggaaattgatgacgggaaagcatgcgggcaacccagtgtataaaactcataaacgtgtaggcagaggctcagctaccagtttggacggctgcttcccaccagcaaagaccacgactggagagccgagccggaggcagctgg

SEQ ID NO: 95 - DNA, 1389 bp, Homo sapiens
  source 1..1389: mol_type = genomic DNA
tggcacacacgcaccctgtccaatgtatcttttgtgtaaatctggacttaacacttcaagcaaactgcctggcttgctgaaaggtggagacacctttcgattcagtcttttaatatgtgttgagtgccacctatgtgcagagcaagatattggggactttggagagatccagaagagtgagaagacagtatcctaccttagggggttcccagtccaatgagggaagcagccccatgccttgggagctcccaagctatagaagcagctaacaatcgagtctggaaaggcaaacaacttcaggacccgcttctaaagcggaatcgcaagtacacgcaaaatgaatccagccttgactgtgtggagttgggtaaaccacctgcctcttacgttgatggggaactagaatgaggacagctccagggaacaagaaagggtagaccataggagctgtcccatgtcccaacagtggggaggagctgatgggcggcccctgctggattagtgttatcctgagaaggcttctggatgcgatgggatttgaggtgctgctgcaaagaatgaattgctcacggaagggtggggtgggggcattccaggtagagggtgcctcctgggggatgcagggaacatgaggggcctgggcaattaatcaagccttgggcacaagcctaggcagtcacccccaattcaaagccagttgaaaatgcagaggagagaggagggccagtgtttggttgtcttgaccaaacccttgaagctggccagcggcaagggcaaggaccagggtcagaggtagagggcgtgagtgaaggcaacccagactgagtccttccctaagcgcccaggtttcctgacagctgttaaggaagcaaggtgagaaagggttaagtgtgcccctccaccgccccaaatgcttcctgtgtttgaaatccttcaggtctctgcaaaccctctggcccccggccaggcgggcattgtccggggagcggttgtaggttgtcagagaggccgcgcagcctttgttgtggggccacctcggggttccctctcgcgctcacgctcgggctggggctgcagagtgcgtgcctggaggggggcggtgcgggaggctcgctccctctccctcttcctgccccccctctagccctcccgatgaccacatgaccaagtgggctcgcggccaagccacaagctacaaaatgcagcccctggagtgagcggggagcattctctctggcagccggggtcacgggcagttgcagccgcggccgagcagccagccgctaagaaagagctcgccgctgccgctcccggagccgccgaggccagcttcgcggcgctgccccgcggcgggagaggaggctgcagaagagcggaggcggccagcgg

SEQ ID NO: 96 - DNA, 4258 bp, synthetic construct
  misc_feature 1..4258: note = Made in Lab - full polynucleotide sequence of vector genome
  source 1..4258: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataagctcctcccagcctcaggcccaggaatgggaatctctgtgggtcacacatcagtagggaggtctttcccgatccttttctatgctactccaggagtcaaagcgtctcctgggacttttcagggcgcttcagaagagccctgggcctaaaccagctcaaccaagctgcagggacccagcctcctgagaaaagtgaatgtgagcccggtgcattcagaggagaatgaagccttcacccagaacacactctgggaagatgtcccaggcccagggggagggtttgtactaccagacctaagtcacctaaactgacaccaagtctcatccatcccaaccattccattccgggtcagaggggtcatcgatttaaccagcaaggctgcccatccaacggttgctccctctgctccctggaagggcctcctcgtgggcgttctgtacctacaggtcttgttccgttctgggaactgccagtggtggcaagaggtggagcaacgggtgccagggcagggagaggtgagtctgggagggaagcagaggcaagatccatggggctttagagactttgccaaagcagtgcgactgctcccaggttgttgtcagccgtcaagagtgagtgcacctccctgggcagacttctgctgccccagtgcccaggaataggcaggggtttgccgcaaaatgaatgacacctggcagacaataagctgaagctttcattagcagcttaagctgaggactatctatgcaaccgatactccctgtgtgctccccgggactgcttaatgtgagcccttgtggagcgattggcaccaagaaagcaaggactaagtcagaagttcaagtcccagccttgccacagcctcagggtgccctcgagcacagcaagcctcagttttcccatctgtacaatgagagaggtacacaaggtagactcgaaggctctttgttgccagggccctgtgttcctttgagtgtatgtgcttctcaggcccacagaggtcctttgtgtttcgtatgtgaactgctctctaggaaacccatgtaactgtctgtgtcctggggcacatacatgaggactcatgtgggccgtattgtgtgtttgtgccggggggaggggagaccccagaacaatgtcccccaccccacccccctcctcaataggcggaagccactggcttcctccctttcctgcctcctgcctcctttgtgccagcaagactgagtactggagagagacaggggatgggaaaaatcagtccagctgtccccaggtctgcccttaccataaccttccccccacctcaagtgactcctcccaggccacacccatccccagccttgtgggggccagattggggggcctagaggctcaaaggcagaatgagtcctcccaccccctaccctgccacccctcccacccaagccacctcatttcctcttcctccccagcaccgacccacactgaccaacacaggctgagcagtcaggcccacagcatctgaccccaggcccagctcgtcctggctggcctgggtcggcctctggagtgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 97 - DNA, 3922 bp, synthetic construct
  misc_feature 1..3922: note = Made in Lab - part of expression cassette
  source 1..3922: mol_type = other DNA
ctctggagacgcgttacataagctcctcccagcctcaggcccaggaatgggaatctctgtgggtcacacatcagtagggaggtctttcccgatccttttctatgctactccaggagtcaaagcgtctcctgggacttttcagggcgcttcagaagagccctgggcctaaaccagctcaaccaagctgcagggacccagcctcctgagaaaagtgaatgtgagcccggtgcattcagaggagaatgaagccttcacccagaacacactctgggaagatgtcccaggcccagggggagggtttgtactaccagacctaagtcacctaaactgacaccaagtctcatccatcccaaccattccattccgggtcagaggggtcatcgatttaaccagcaaggctgcccatccaacggttgctccctctgctccctggaagggcctcctcgtgggcgttctgtacctacaggtcttgttccgttctgggaactgccagtggtggcaagaggtggagcaacgggtgccagggcagggagaggtgagtctgggagggaagcagaggcaagatccatggggctttagagactttgccaaagcagtgcgactgctcccaggttgttgtcagccgtcaagagtgagtgcacctccctgggcagacttctgctgccccagtgcccaggaataggcaggggtttgccgcaaaatgaatgacacctggcagacaataagctgaagctttcattagcagcttaagctgaggactatctatgcaaccgatactccctgtgtgctccccgggactgcttaatgtgagcccttgtggagcgattggcaccaagaaagcaaggactaagtcagaagttcaagtcccagccttgccacagcctcagggtgccctcgagcacagcaagcctcagttttcccatctgtacaatgagagaggtacacaaggtagactcgaaggctctttgttgccagggccctgtgttcctttgagtgtatgtgcttctcaggcccacagaggtcctttgtgtttcgtatgtgaactgctctctaggaaacccatgtaactgtctgtgtcctggggcacatacatgaggactcatgtgggccgtattgtgtgtttgtgccggggggaggggagaccccagaacaatgtcccccaccccacccccctcctcaataggcggaagccactggcttcctccctttcctgcctcctgcctcctttgtgccagcaagactgagtactggagagagacaggggatgggaaaaatcagtccagctgtccccaggtctgcccttaccataaccttccccccacctcaagtgactcctcccaggccacacccatccccagccttgtgggggccagattggggggcctagaggctcaaaggcagaatgagtcctcccaccccctaccctgccacccctcccacccaagccacctcatttcctcttcctccccagcaccgacccacactgaccaacacaggctgagcagtcaggcccacagcatctgaccccaggcccagctcgtcctggctggcctgggtcggcctctggagtgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttc

SEQ ID NO: 98 - DNA, 3850 bp, synthetic construct
  misc_feature 1..3850: note = Made in Lab - full polynucleotide sequence of vector genome
  source 1..3850: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataaagcttccgaccgttagtcagagaactgtaagtgctcagagcctggctgacaatgatctggaatgaaccagataacaacataataaaatctcagtaaaataatttaacagttagcttggaagctggtcagctctggggaaatcagggtaaattgtgctgtcatgaactgtcccacactgacatcggccaaagtgaatatgaactttggtagatccaatgcctgttctatttatttttccagtgaaaagtattttgatagagcttttcattttgtaaatacactgagttaaccaaaatatcatggatttccgtttgttcttaagacatgcaactcgtctacggctataccactctgaacgcgcccgatctcggaagacatgcaactcaaatgtaaatacagtagaatattacttaggtagaaactcctggtgattttaaaagattggaaaagaatatgaggaagagttgaataatgcaaattctagtgtgtgtgctaccgaagtgaacacttaatgcacagtctacagactaggacattttatcgtgtgttgtaaaattgggtagaaacttgtgtttgtgaaaactgagcattaaaaccttacagagaccgtttcttgtttacttttgaaaaaaaaaagagtcacgtgagcctcattttgtatttgtgtgtgtgtgtgtgtgtgtgtctcccctcctcccagcgtgtgtgtgctgggaggaggggagaccccagaacaatgtcctgcctccaaaccttctcaataggcggaagccactggcttcctccctttcctgtctcccgtgctccagcaatgcagatggaagggaccgaagggatgggagagagagcccaaccatccccagatctgtccttgtcacaacctgcctcccacctctaatgcccccccttccagagacttccaggccacacccatcccgggcttgtgggggctggacacgggaggactacaggcgacaactcttcccaccctctctccctgccacccctcctaccctaaccatcatttcctcttcctccccagcaccgaggtgcactgagctggacaggctgaacactcagacccacagcaactgaccccgggcccagctggccttggctggcccagggcagcttccagagtgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 99 - DNA, 3514 bp, synthetic construct
  misc_feature 1..3514: note = Made in Lab - part of expression cassette
  source 1..3514: mol_type = other DNA
ctctggagacgcgttacataaagcttccgaccgttagtcagagaactgtaagtgctcagagcctggctgacaatgatctggaatgaaccagataacaacataataaaatctcagtaaaataatttaacagttagcttggaagctggtcagctctggggaaatcagggtaaattgtgctgtcatgaactgtcccacactgacatcggccaaagtgaatatgaactttggtagatccaatgcctgttctatttatttttccagtgaaaagtattttgatagagcttttcattttgtaaatacactgagttaaccaaaatatcatggatttccgtttgttcttaagacatgcaactcgtctacggctataccactctgaacgcgcccgatctcggaagacatgcaactcaaatgtaaatacagtagaatattacttaggtagaaactcctggtgattttaaaagattggaaaagaatatgaggaagagttgaataatgcaaattctagtgtgtgtgctaccgaagtgaacacttaatgcacagtctacagactaggacattttatcgtgtgttgtaaaattgggtagaaacttgtgtttgtgaaaactgagcattaaaaccttacagagaccgtttcttgtttacttttgaaaaaaaaaagagtcacgtgagcctcattttgtatttgtgtgtgtgtgtgtgtgtgtgtctcccctcctcccagcgtgtgtgtgctgggaggaggggagaccccagaacaatgtcctgcctccaaaccttctcaataggcggaagccactggcttcctccctttcctgtctcccgtgctccagcaatgcagatggaagggaccgaagggatgggagagagagcccaaccatccccagatctgtccttgtcacaacctgcctcccacctctaatgcccccccttccagagacttccaggccacacccatcccgggcttgtgggggctggacacgggaggactacaggcgacaactcttcccaccctctctccctgccacccctcctaccctaaccatcatttcctcttcctccccagcaccgaggtgcactgagctggacaggctgaacactcagacccacagcaactgaccccgggcccagctggccttggctggcccagggcagcttccagagtgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttc

SEQ ID NO: 100 - DNA, 3010 bp, synthetic construct
  misc_feature 1..3010: note = Made in Lab - full polynucleotide sequence of vector genome
  source 1..3010: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtactctggagacgcgttacataaccattttgctagagaaggccgcggaggctcagagaggtgcgcacacttgccctgagtcacacagcgaatgccctccgcggtcccaacgcagagagaacgagccgatcggcagcctgagcgaggcagtggttagggggggccccggccccggccactcccctcaccccctccccgcagagcgccgcccaggacaggctgggccccaggccccgccccgaggtcctgcccacacacccctgacacaccggcgtcgccagccaatggccggggtcctataaacgctacggtccgcgcgctctctgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgcgttacccaggctggagtgcagtggcacatttctgctcactgcaacctcctcctccctgggttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 101 - DNA, 2611 bp, synthetic construct
  misc_feature 1..2611: note = Made in Lab - part of expression cassette
  source 1..2611: mol_type = other DNA
ctctggagacgcgttacataaccattttgctagagaaggccgcggaggctcagagaggtgcgcacacttgccctgagtcacacagcgaatgccctccgcggtcccaacgcagagagaacgagccgatcggcagcctgagcgaggcagtggttagggggggccccggccccggccactcccctcaccccctccccgcagagcgccgcccaggacaggctgggccccaggccccgccccgaggtcctgcccacacacccctgacacaccggcgtcgccagccaatggccggggtcctataaacgctacggtccgcgcgctctctgccaccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgataatggatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcattgcctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 102 - DNA, 302 bp, Homo sapiens
  source 1..302: mol_type = genomic DNA
accattttgctagagaaggccgcggaggctcagagaggtgcgcacacttgccctgagtcacacagcgaatgccctccgcggtcccaacgcagagagaacgagccgatcggcagcctgagcgaggcagtggttagggggggccccggccccggccactcccctcaccccctccccgcagagcgccgcccaggacaggctgggccccaggccccgccccgaggtcctgcccacacacccctgacacaccggcgtcgccagccaatggccggggtcctataaacgctacggtccgcgcgctctct

SEQ ID NO: 103 - DNA, 2235 bp, synthetic construct
  misc_feature 1..2235: note = Synthetic AAV-BR1 capsid coding sequence
  source 1..2235: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggccagagaggcaatcgggggactgagtgggatgcccaggcggccaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 104 - protein, 744 aa, synthetic construct
  REGION 1..744: note = Synthetic AAV-BR1 VP1 sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGQRGNRGTEWDAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 105 - protein, 607 aa, synthetic construct
  REGION 1..607: note = Synthetic AAV-BR1 VP2 sequence
TAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGQRGNRGTEWDAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 106 - protein, 542 aa, synthetic construct
  REGION 1..542: note = Synthetic AAV-BR1-VP3 sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGQRGNRGTEWDAQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 107 - skipped

SEQ ID NO: 108 - protein, 7 aa, synthetic construct
  REGION 1..7: note = AAV-BR1 insert sequence
ADGVQWT

SEQ ID NO: 109 - protein, 7 aa, synthetic construct
  REGION 1..7: note = AAV-BR1 insert sequence
DDGVSWK

SEQ ID NO: 110 - protein, 7 aa, synthetic construct
  REGION 1..7: note = AAV-BR1 insert sequence
SDGLTWS

SEQ ID NO: 111 - protein, 7 aa, synthetic construct
  REGION 1..7: note = AAV-BR1 insert sequence
SDGLAWV

SEQ ID NO: 112 - protein, 7 aa, synthetic construct
  REGION 1..7: note = AAV-BR1 insert sequence
NRGTEWD

(19) United States
(12) Patent Application Publication
(10) Pub. No.: US 2025/0135040 A1
(43) Pub. Date: May 1, 2025

(54) ADENO-ASSOCIATED VIRAL VECTOR FOR GLUT1 EXPRESSION AND USES THEREOF

(71) Applicant: SPACECRAFT SEVEN, LLC, Cranbury, NJ (US)

(72) Inventors: Christopher Dean HERZOG, Cranbury, NJ (US); Chester Bittencort SACRAMENTO, Cranbury, NJ (US); Raj PRABHAKAR, Cranbury, NJ (US)

(21) Appl. No.: 18/837,053

(22) PCT Filed: Feb. 8, 2023
(86) PCT No.: PCT/US2023/062231

§ 371 Date: Aug. 8, 2024

Related U.S. Application Data
(60) Provisional application No. 63/307,963, filed on Feb. 8, 2022.

(51) Int. Cl. A61K 48/00; A61K 38/17; A61P 3/00; A61P 25/00; C12N 15/86

(52) U.S. Cl. CPC A61K 48/0058; A61K 38/1709; A61K 48/0075; A61K 48/0083; A61P 3/00; A61P 25/00; C12N 15/86; C12N 2750/14122; C12N 2750/14143; C12N 2830/48; C12N 2830/50

(57) ABSTRACT

Provided herein is a gene therapy for GLUT1 Deficiency Syndrome and related disorders using a recombinant adeno-associated virus (rAAV) virion as a vector to express an GLUT1 protein or functional variant thereof. The capsid may be an AAV-BR1 capsid or a functional variant thereof. Other promoters or capsids may be used. The rAAV virion may use an endothelial-specific promoter, e.g., a FLT-1 promoter. Further provided are methods of treatment, such as by intracerebrally and/or intravenously of the rAAV virion, and other compositions and methods.

### CROSS-REFERENCE TO RELATED APPLICATIONS

This application claims benefit of priority to U.S. Provisional Patent Application No. 63/307,963, filed Feb. 8, 2022, the disclosure of which is incorporated herein by reference in its entirety for all purposes.

### STATEMENT REGARDING THE SEQUENCE LISTING

The Sequence Listing XML associated with this application is provided in XML file format and is hereby incorporated by reference into the specification. The name of the XML file containing the Sequence Listing XML is ROPA_027_01WO_ST26.xml. The XML file is 223m012 bytes, and created on Feb. 8, 2023, and is being submitted electronically via USPTO Patent Center.

### BACKGROUND

Mutations in the SLC2A1 gene encoding glucose transporter 1 (GLUT1) are associated with a neurodevelopmental disorder termed GLUT1 Deficiency Syndrome (GLUT1 DS). GLUT1 DS is an autosomal-dominant disorder which is often presents as a sporadic disease with de novo mutations producing haploinsufficiency and conferring symptomatic heterozygosity.

GLUT1 is an insulin-independent glucose transporter. Patients with classic GLUT1 DS, also known as De Vivo disease, suffer low brain glucose levels and exhibit a phenotype characterized by: early-onset seizures (median 12 months), delayed development, acquired microcephaly (decelerating head growth), complex movement disorders (spasticity, ataxia, dystonia); paroxysmal eye-head movements; and hypoglycorrhachia, or low glucose concentration in cerebrospinal fluid (CSF). The clinical course of the disease reveals the importance of early treatment. Alter et al. J. Child Neurol. 30(2):160-169 (2015). GLUT1 has been implicated in the function of endothelial cells, including transport of glucose across the blood-brain barrier (BBB), angiogenesis and maintenance of the BBB. However, studies in haploinsufficient mouse models have provided conflicting evidence concerning the role of GLUT1 in maintaining the physical integrity of the BBB. Although an endothelial cell lineage-specific knockout of GLUT1 reduces endothelial energy availability and reduces proliferation without affecting migration, thereby delaying developmental angiogenesis (Veys et al., Circ. Res. 2020; 127:466-482), the effect of restoring GLUT1 expression specifically in endothelial cells has not been tested.

Therapeutic strategies for the disease are reviewed in Tang et al. Ann. Clin. Trans. Neurol. 2019; 6(9):1923-1932. Current standard of care is ketogenic diet, which raises the levels of ketones, which substitute for glucose, in the blood to make them available to the brain. Treatment with the triglyceride Triheptanoin has been proposed as an alternative to ketogenic diet. Gene therapy using adeno-associated virus (AAV) vectors have also been attempted. Targeting GLUT1 deficiency in neurons, AAV9 vectors encoding GLUT1 under the control of a neuron-specific promoter (e.g., synapsin) have been tested in a young postnatal mouse model. Other studies employed a constitutive promoter (e.g., CMV promoter) or the promoter of the endogenous GLUT1 gene. Various small molecules have also been tested, including the anticonvulsant carbonic anhydrase inhibitor acetazolamide and others.

While haploinsufficiency of GLUT1 arrests brain angiogenesis resulting in a relatively diminutive cerebral microvasculature, which may be related to glucose-dependence of endothelial tip cells, Tang et al. have observed that whether low GLUT1 in endothelial cells triggers this pathology remains to be investigated. The GLUT1 protein is expressed in additional brain cells including oligodendrocytes, microglia, and ependymal cells.

There is an unmet need for therapy for GLUT1 Deficiency Syndrome. The gene therapies provided herein address this need.

### SUMMARY

The present invention relates generally to gene therapy for neurological disease or disorders using adeno-associated virus (AAV)-based delivery of a polynucleotide encoding GLUT1 or a functional variant thereof.

Although GLUT1 Deficiency Syndrome (DS) is a neurodevelopmental disorder with clinical manifestations rooted in lack of appropriate neuronal function, the present gene therapy may, without being bound by theory, target endothelial cells responsible for guiding the angiogenesis and development of the vasculature in the central nervous system (CNS). Delivery of AAV to the developing central nervous system CNS vasculature either via intravenous, direct delivery to intracerebroventricular system, or by a combination of both routes, with subsequent GLUT1 protein expression in endothelial tip cells, may promote vascular growth and formation throughout the CNS during a critical window of angiogenesis and neurodevelopment. In addition, delivery of AAV to the CNS vasculature, with subsequent GLUT1 protein expression in mature endothelial cells may promote increased availability of glucose to neurons by increasing the transport of glucose across the blood-brain barrier.

In one aspect, the disclosure provides a recombinant adeno-associated virus (rAAV) virion, comprising a vector genome and a capsid, wherein the vector genome comprises an expression cassette, flanked by 5′ and 3′ inverted terminal repeats (ITRs), wherein the expression cassette comprises a polynucleotide sequence encoding GLUT1 or a functional variant thereof, operatively linked to a promoter, and wherein the capsid is a BR1 capsid or a functional variant thereof.

In some embodiments, the capsid is a BR1 capsid.

In some embodiments, the capsid comprises the polypeptide sequence motif XDGXXWX, wherein each X is any amino acid (SEQ ID NO: 107).

In some embodiments, the capsid comprises the polypeptide sequence ADGVQWT (SEQ ID NO:108), DDGVSWK (SEQ ID NO:109), SDGLTWS (SEQ ID NO:110) or SDGLAWV (SEQ ID NO:111).

In some embodiments, the capsid comprises the polypeptide sequence NRGTEWD or a functional variant having 1, 2, 3, or more substitutions thereto.

In some embodiments, the capsid comprises the polypeptide sequence NRGTEWD (SEQ ID NO:112).

In some embodiments, the capsid comprises an insertion of the polypeptide sequence NRGTEWD in the GH loop compared to an AAV2 VP1 reference sequence as set forth in SEQ ID NO 76.

In some embodiments, the capsid comprises a VP3 polypeptide that shares at least 98%, at least 99%, or 100% identity to an AAV2 VP3 polypeptide sequence as set forth in SEQ ID NO: 106.

In some embodiments, the capsid comprises a VP2 polypeptide that shares at least 98%, at least 99%, or 100% identity to an AAV2 VP2 polypeptide sequence as set forth in SEQ ID NO: 105.

In some embodiments, the capsid comprises a VP1 polypeptide that shares at least 98%, at least 99%, or 100% identity to an AAV2 VP1 polypeptide sequence as set forth in SEQ ID NO: 104.

In some embodiments, the promoter is a FLT-1 promoter.

In some embodiments, the FLT-1 promoter is a human FLT-1 (hFLT-1) promoter.

In some embodiments, the hFLT-1 promoter shares at least 75%, at least 80%, at least 85%, at least 90%, at least 95%, at least 96%, at least 97%, at least 98%, at least 99% or 100% identity with SEQ ID NO: 1.

In some embodiments, the expression cassette comprises a polyA signal, optionally a human growth hormone (hGH) polyA.

In some embodiments, the expression cassette comprises a Woodchuck Hepatitis Virus Posttranscriptional Regulatory Element (WPRE), optionally a WPRE(x).

In some embodiments, the expression cassette comprises a 3′ untranslated region (3′ UTR) comprising a sequence that shares at least 75%, at least 80%, at least 85%, at least 90%, at least 95%, at least 96%, at least 97%, at least 98%, at least 99% or 100% identity with SEQ ID NO: 4.

In some embodiments, the polynucleotide sequence encoding GLUT1 is a SLC2A1 polynucleotide.

In some embodiments, the SLC2A1 polynucleotide is a human SLC2A1 polynucleotide.

In some embodiments, the polynucleotide sequence encoding GLUT1 shares at least 75%, at least 80%, at least 85%, at least 90%, at least 95%, at least 96%, at least 97%, at least 98%, at least 99% or 100% identity with SEQ ID NO: 5.

In some embodiments, the expression cassette is flanked by 5′ and 3′ inverted terminal repeats (ITRs), optionally AAV2 ITRs, optionally an ITR that shares at least 75%, at least 80%, at least 85%, at least 90%, at least 95%, at least 96%, at least 97%, at least 98%, at least 99% or 100% identity with SEQ ID NO: 6 or SEQ ID NO: 7.

In some embodiments, the expression cassette shares at least 75%, at least 80%, at least 85%, at least 90%, at least 95%, at least 96%, at least 97%, at least 98%, at least 99% or 100% identity with any one of SEQ ID NOs: 8-16, SEQ ID NO: 97, SEQ ID NO: 99, and SEQ ID NO: 101.

In some embodiments, the rAAV virion is not an AAV2 virion.

In some embodiments, the disclosure provides a method of treating and/or preventing a disease or disorder in a subject in need thereof, comprising administering the rAAV virion to the subject.

In some embodiments, the disease or disorder is a neurological disorder.

In some embodiments, the disease or disorder is Glucose transporter 1 deficiency syndrome (GLUT IDS) or De Vivo Disease.

In some embodiments, the rAAV virion is administered by intracerebroventricular (ICV) injection.

In some embodiments, the rAAV virion is administered by an intravenous (IV) injection.

In some embodiments, the rAAV virion is administered by ICV injection in combination with an IV injection.

In some embodiments, the administration results in expression of the polynucleotide sequence encoding GLUT1 in the brain, optionally at increased levels compared to a reference rAAV virion.

In some embodiments, the administration results in an increase in expression of GLUT1 protein in the brain and/or an increase in glucose levels and/or lactate levels in the CSF, optionally at increased levels compared to a reference rAAV virion, wherein optionally the increases is an increase of at least about 10%, at least about 20%, at least about 30%, at least about 40%, at least about 50%, at least about 60%, at least about 70%, at least about 80%, at least about 90%, or 100%.

In some embodiments, the reference rAAV virion is a variant of an AAV2 virion.

In some embodiments, the reference rAAV virion is an AAV-BR1 virion.

In some embodiments, the rAAV virion is administered at a dose of 1×10¹² vector genomes (vg), 1×10¹³ vg, 1×10¹⁴ vg, or 3×10¹⁴ vg.

In some embodiments, the method causes increased glucose uptake by cerebral microvasculature endothelial cells compared to a method performed using an endogenous Glut1 promoter or a ubiquitous promoter.

In some embodiments, the disclosure provides a method of expressing GLUT1 in a cell, comprising contacting the cells with the rAAV virion.

In some embodiments, the cell is an endothelial cell.

In some embodiments, the endothelial cell is a cerebral microvasculature endothelial cell.

In some embodiments, the endothelial cell is an in vivo endothelial cell.

In some embodiments, the cell is a neuron.

In some embodiments, the neuron is an in vivo neuron.

In some embodiments, the method comprises in vivo administration of the rAAV virion to a subject.

In some embodiments, the rAAV virion causes increased glucose uptake by the cell compared to a cell contacted with a rAAV virion comprising an endogenous Glut1 promoter or a ubiquitous promoter.

In some embodiments, the disclosure provides a pharmaceutical composition comprising the rAAV virion.

In some embodiments, the disclosure provides a kit comprising the rAAV virion or a pharmaceutical composition and optionally instructions for use.

In further aspects, the disclosure provides polynucleotides (e.g., vector genomes), pharmaceutical compositions, kits, and other compositions and methods.

Various other aspects and embodiments are disclosed in the detailed description that follows. The invention is limited solely by the appended claims.

### BRIEF DESCRIPTION OF FIGURES

FIG. 1 shows a vector diagrams for various non-limiting examples of a vector genome.

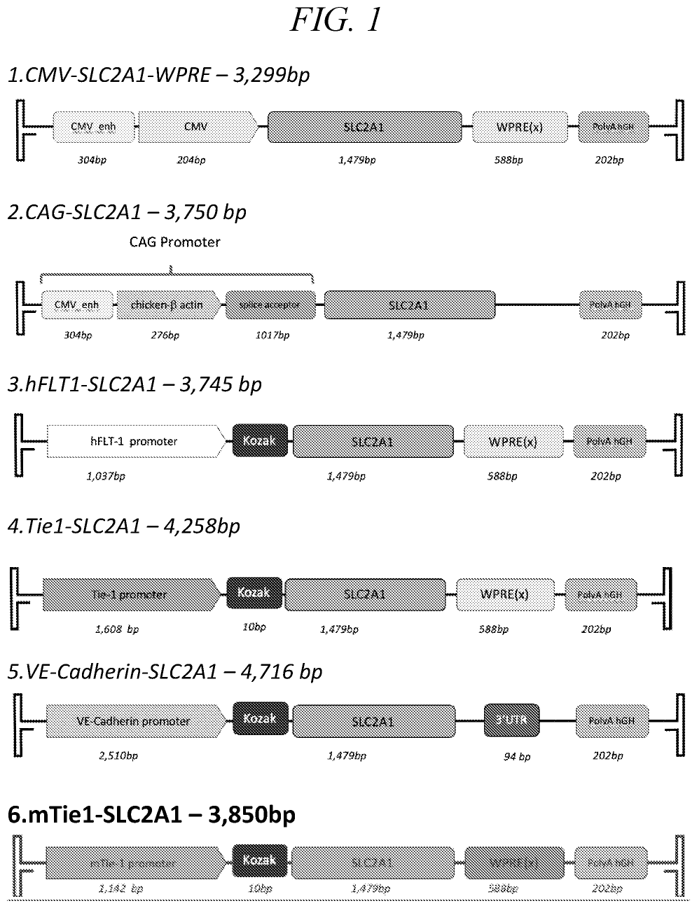

FIG. 2 shows a vector diagram of a non-limiting example of a vector genome. The full polynucleotide sequence of the vector genome is SEQ ID NO: 17. The capitalized portion is the expression cassette (SEQ ID NO: 8).

FIG. 3 shows a vector diagram of a non-limiting example of a vector genome. The full polynucleotide sequence of the vector genome is SEQ ID NO: 19. The capitalized portion is the expression cassette (SEQ ID NO: 10).

FIG. 4 shows a vector diagram of a non-limiting example of a vector genome. The full polynucleotide sequence of the vector genome is SEQ ID NO: 21. The capitalized portion is the expression cassette (SEQ ID NO: 12).

FIG. 5 shows a vector diagram of a non-limiting example of a vector genome. The full polynucleotide sequence of the vector genome is SEQ ID NO: 96. The capitalized portion is the expression cassette (SEQ ID NO: 97). An alternative of the full polynucleotide sequence of the vector genome is SEQ ID NO: 23. An alternative of the expression cassette is SEQ ID NO: 14.

FIG. 6 shows a vector diagram of a non-limiting example of a vector genome. The full polynucleotide sequence of the vector genome is SEQ ID NO: 25. The capitalized portion is the expression cassette (SEQ ID NO: 16).

FIG. 7 shows a vector diagram of a non-limiting example of a vector genome. The full polynucleotide sequence of the vector genome is SEQ ID NO: 98. The capitalized portion is the expression cassette (SEQ ID NO: 99).

FIG. 8 shows a vector diagram of a non-limiting example of a vector genome. The full polynucleotide sequence of the vector genome is SEQ ID NO: 100. The capitalized portion is the expression cassette (SEQ ID NO: 101).

FIG. 9. AAV9-mediated Expression of hGlut1 protein CHO-Lec2 Cells. CHO-Lec2 cells were transduced with AAV9 vectors expressing the hGlut1 transgene protein driven by one of several endothelial-specific promoters (i.e., hFLT1, mTie1 or hGlut1) or by the ubiquitous CMV promoter. [SLC2A1=GLUT1 Gene].

FIGS. 10A-10C. Expression of transgene protein (Glut1-GFP) following transfection of human cerebral microvasculature endothelial cells (hCMEC/d3s).

FIG. 10A. GFP fluorescence 72 hours following transfection with constructs containing one of several endothelial cell promoters driving expression of Glut1-GFP transgene.

FIG. 10B. GFP fluorescence 72 hours following transfection with constructs containing one of two ubiquitous promoters (CMV or CAG), control vector without Glut1 (CMV-GFP) or no transfection (No NFX). Images obtained using Operetta CLS™ (PerkinElmer®).

FIG. 10C. Diagram of expression cassette containing the promoter of interest (hFLT1, mTie, hTie or hGlut1) and the GLUT1 (SLC2A1) gene (T2A linked-GFP) and regulatory elements flanked by AAV2 inverted terminal repeats (ITRs).

FIGS. 11A-11C. 2-Deoxy-D-glucose (glucose) Uptake in hCMEC/d3 cells following expression of human GLUT1 (SLC2A1). Human cerebromicrovascular endothelial cells (hCMEC/d3s) were transfected with plasmids expressing either CAG-GFP (negative control) or with a hGLUT1-t2A-eGFP transgene driven by one of several endothelial-specific promoters (i.e., hFLT1, mTie1 or hGlut1) or by the ubiquitous CMV promoter. Glucose uptake was measured using a luminescence-based kit (Promega®) with 0.5 mM 2-Deoxy-D-glucose (2-DG) in culture media. Glucose uptake was normalized by Total Cells using phase-contrast imaging [Error bars represent S.E.M; n=6 replicates per condition].

FIG. 11A. Glucose (2-DG) uptake was measured at 72 hours post-transfection in a first experiment.

FIG. 11B. Glucose (2-DG) uptake was measured at 72 hours post-transfection in a second experiment.

FIG. 11C. Glucose (2-DG) uptake was measured at 96 hours post-transfection.

FIGS. 12A-12B. 2-Deoxy-D-glucose (glucose) Uptake in hCMEC/d3 cells following expression of human GLUT1 (SLC2A1). Human cerebromicrovascular endothelial cells (hCMEC/d3s) were transfected with plasmids expressing a hGLUT1-t2A-eGFP transgene driven by one of several endothelial-specific promoters (i.e., hFLT1, mTie1 or hGlut1) or by the ubiquitous CMV promoter. Non-transfected hCMEC/d3 served as controls (CON). Glucose uptake was measured using a luminescence-based kit (Promega®) with varying concentrations (0 mM, 0.1 mM, 0.5 mM or 1.0 mM) of 2-Deoxy-D-glucose in the culture media. Glucose uptake was normalized on a per cell basis through multiplexing with the RealTime-Glo MT Cell Viability Assay (Promega®), performed according to the manufacturer's recommendations.

FIG. 12A. shows glucose uptake in hCMEC/d3 cells following expression of human Glut1 (SLC2A1) at a 72-hour time point.

FIG. 12B. shows glucose uptake in hCMEC/d3 cells following expression of human Glut1 (SLC2A1) at a 96-hour time point.

FIG. 13. 2-Deoxy-D-glucose (glucose) Uptake Following AAV9-mediated Expression of hGLUT1 (SLC2A1) in hCMEC/d3 Cells. Human cerebromicrovascular endothelial cells (hCMEC/d3s) were transduced with AAV9 vectors (3×10⁵ vector genomes/cell) expressing either CAG-GFP (negative control) or the hGLUT1 transgene driven by one of several endothelial-specific promoters (i.e., hFLT1, mTie1 or hGlut1) or by the ubiquitous CMV promoter. Glucose (2-DG) uptake was measured 72 hours post-transduction using the luminescence-based Glucose Uptake-Glo kit (Promega®) and normalized per cell using the RealTime-Glo MT Cell Viability Assay (Promega®) [Error bars represent S.E.M; n=4 replicates per condition].

### DETAILED DESCRIPTION OF THE INVENTION

The present disclosure provided gene therapy vectors for GLUT1 that deliver a polynucleotide encoding GLUT1 or a functional variant thereof with an AAV virion having tropism for endothelial cells, such as AAV-BR1, along with methods of use, and other compositions and methods. In some embodiments, the AAV vector genome includes an endothelial cell-specific promoter, such as an FLT-1 promoter.

This disclosure further provides methods of treating a disease or disorder in a subject by administering a gene therapy vector of the disclosure. In some embodiments, the disease or disorder is Glucose transporter 1 deficiency syndrome (GLUT1 DS) or De Vivo Disease.

In accordance with the present invention, a polynucleotide encoding a GLUT1 or functional variant thereof may be employed in generating a gene therapy vector. The resulting vector may be employed in treating diseases or disorders, e.g. Glucose transporter 1 deficiency syndrome (GLUT1 DS), De Vivo Disease or others.

### Definitions

The section headings are for organizational purposes only and are not to be construed as limiting the subject matter described to particular aspects or embodiments.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The term “sequence identity” refers to the percentage identity of a polypeptide or polynucleotide sequence of interest to a reference sequence, calculated as 100 times the number of exact matches in an optimal alignment of the sequence of interest to the reference sequence divided by the total length of the reference sequence (including gaps). An optimal alignment of the sequences may be generated using the European Molecular Biology Open Software Suite (EMBOSS) needle program available at www.ebi.ac.uk, as described in Maderia et al. Nucleic Acids Res. 47(W1): W636-W641 (2019). When comparing DNA and RNA sequences, thymine (T) and uracil (U) are counted as a match.

As used herein, an “AAV vector” or “rAAV vector” refers to a recombinant vector comprising one or more polynucleotides of interest (or transgenes) that are flanked by AAV terminal repeat sequences (ITRs). Such AAV vectors can be replicated and packaged into infectious viral particles when present in a host cell that has been transfected with a plasmid encoding and expressing rep and cap gene products. Alternatively, AAV vectors can be packaged into infectious particles using a host cell that has been stably engineered to express rep and cap genes.

As used herein, an “AAV virion” or “AAV viral particle” or “AAV vector particle” refers to a viral particle composed of at least one AAV capsid protein and an encapsidated polynucleotide AAV vector. As used herein, if the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

As used herein, “GH loop” refers to the loop created between the G and H strands of the jelly-roll β-barrel of the AAV capsid protein VP1, as described in Xie et al. PNAS 99(16):10405-10410 (2002).

As used herein, “promoter” refers to a polynucleotide sequence capable of promoting initiation of RNA transcription from a polynucleotide in a eukaryotic cell.

As used herein, “vector genome” refers to the polynucleotide sequence packaged by the vector (e.g., an rAAV virion), including flanking sequences (in AAV, inverted terminal repeats). The terms “expression cassette” and “polynucleotide cassette” refer to the portion of the vector genome between the flanking ITR sequences. “Expression cassette” implies that the vector genome comprises at least one gene encoding a gene product operably linked to an element that drives expression (e.g., a promoter).

As used herein, the term “patient in need” or “subject in need” refers to a patient or subject at risk of, or suffering from, a disease, disorder or condition that is amenable to treatment or amelioration with a recombinant gene therapy vector or gene editing system disclosed herein. A patient or subject in need may, for instance, be a patient or subject diagnosed with a disorder associated with central nervous system. A subject may have a mutation in an SLC2A1 gene or deletion of all or a part of SLC2A1 gene, or of gene regulatory sequences, that causes aberrant expression of the GLUT1 protein. “Subject” and “patient” are used interchangeably herein. The subject treated by the methods described herein may be an adult or a child. Subjects may range in age.

As used herein, the term “variant” or “functional variant” refer, interchangeably, to a protein that has one or more amino-acid substitutions, insertions, or deletions compared to a parental protein that retains one or more desired activities of the parental protein.

As used herein, “genetic disruption” refers to a partial or complete loss of function or aberrant activity in a gene. For example, a subject may suffer from a genetic disruption in expression or function in the SLC2A1 gene that decreases expression or results in loss or aberrant function of the GLUT1 protein in at least some cells (e.g., endothelial cells and/or neurons) of the subject.

As used herein, “treating” refers to ameliorating one or more symptoms of a disease or disorder. The term “preventing” refers to delaying or interrupting the onset of one or more symptoms of a disease or disorder or slowing the progression of SLC2A-related neurological disease or disorder, e.g., GLUT1 Deficiency Syndrome (GLUT1 DS).

### GLUT1 Protein or Polynucleotide

The present disclosure contemplates compositions and methods of use related to glucose transporter 1 (GLUT1) protein. Various mutations in SLC2A1 are known to be associated with GLUT1 DS. Both inherited and de novo mutations have been observed. In some cases, a heterozygous missense mutation is sufficient to cause disease.

The polypeptide sequence of GLUT1 is as follows:

(SEQ ID NO: 26)
MEPSSKKLTGRLMLAVGGAVLGSLOFGYNTGVINAPQKVIEEFYNQTWVH
RYGESILPTTLTTLWSLSVAIFSVGGMIGSFSVGLFVNRFGRRNSMLMMN
LLAFVSAVLMGFSKLGKSFEMLILGRFIIGVYCGLTTGFVPMYVGEVSPT
ALRGALGTLHQLGIVVGILIAQVFGLDSIMGNKDLWPLLLSIIFIPALLQ
CIVLPFCPESPRELLINRNEENRAKSVLKKLRGTADVTHDLQEMKEESRQ
MMREKKVTILELFRSPAYRQPILIAVVLOLSQQLSGINAVFYYSTSIFEK
AGVQQPVYATIGSGIVNTAFTVVSLFVVERAGRRTLHLIGLAGMAGCAIL
MTIALALLEQLPWMSYLSIVAIFGFVAFFEVGPGPIPWFIVAELFSQGPR
PAAIAVAGFSNWTSNFIVGMCFQYVEQLCGPYVFIIFTVLLVLFFIFTYF
KVPETKGRTFDEIASGFRQGGASQSDKTPEELFHPLGADSQV.

In some embodiments, the GLUT1 protein comprises a polypeptide sequence at least 75%, at least 80%, at least 85%, at least 90%, at least 92%, at least 93%, at least 94%, at least 95%, at least 96%, at least 97%, at least 98%, at least 99%, or 100% identical to SEQ ID NO: 26).

In some embodiments, the disclosure provides a recombinant adeno-associated virus (rAAV) virion, comprising a capsid and a vector genome, wherein the vector genome comprises a polynucleotide sequence encoding the GLUT1 protein or a functional variant thereof, operatively linked to a promoter. In some embodiments, the disclosure provides a recombinant adeno-associated virus (rAAV) virion, comprising a capsid and genome, wherein the rAAV virion genome comprises a polynucleotide sequence encoding an GLUT1 protein, operatively linked to a promoter. The polynucleotide encoding the GLUT1 protein may comprise a polynucleotide sequence at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 5)
ATGGAGCCCAGCAGCAAGAAGCTGACGGGTCGCCTCATGCTGGCCGTGGG
AGGAGCAGTGCTTGGCTCCCTGCAGTTTGGCTACAACACTGGAGTCATCA
ATGCCCCCCAGAAGGTGATCGAGGAGTTCTACAACCAGACATGGGTCCAC
CGCTATGGGGAGAGCATCCTGCCCACCACGCTCACCACGCTCTGGTCCCT
CTCAGTGGCCATCTTTTCTGTTGGGGGCATGATTGGCTCCTTCTCTGTGG
GCCTTTTCGTTAACCGCTTTGGCCGGCGGAATTCAATGCTGATGATGAAC
CTGCTGGCCTTCGTGTCCGCCGTGCTCATGGGCTTCTCGAAACTGGGCAA
GTCCTTTGAGATGCTGATCCTGGGCCGCTTCATCATCGGTGTGTACTGCG
GCCTGACCACAGGCTTCGTGCCCATGTATGTGGGTGAAGTGTCACCCACA
GCCCTTCGTGGGGCCCTGGGCACCCTGCACCAGCTGGGCATCGTCGTCGG
CATCCTCATCGCCCAGGTGTTCGGCCTGGACTCCATCATGGGCAACAAGG
ACCTGTGGCCCCTGCTGCTGAGCATCATCTTCATCCCGGCCCTGCTGCAG
TGCATCGTGCTGCCCTTCTGCCCCGAGAGTCCCCGCTTCCTGCTCATCAA
CCGCAACGAGGAGAACCGGGCCAAGAGTGTGCTAAAGAAGCTGCGCGGGA
CAGCTGACGTGACCCATGACCTGCAGGAGATGAAGGAAGAGAGTCGGCAG
ATGATGCGGGAGAAGAAGGTCACCATCCTGGAGCTGTTCCGCTCCCCCGC
CTACCGCCAGCCCATCCTCATCGCTGTGGTGCTGCAGCTGTCCCAGCAGC
TGTCTGGCATCAACGCTGTCTTCTATTACTCCACGAGCATCTTCGAGAAG
GCGGGGGTGCAGCAGCCTGTGTATGCCACCATTGGCTCCGGTATCGTCAA
CACGGCCTTCACTGTCGTGTCGCTGTTTGTGGTGGAGCGAGCAGGCCGGC
GGACCCTGCACCTCATAGGCCTCGCTGGCATGGCGGGTTGTGCCATACTC
ATGACCATCGCGCTAGCACTGCTGGAGCAGCTACCCTGGATGTCCTATCT
GAGCATCGTGGCCATCTTTGGCTTTGTGGCCTTCTTTGAAGTGGGTCCTG
GCCCCATCCCATGGTTCATCGTGGCTGAACTCTTCAGCCAGGGTCCACGT
CCAGCTGCCATTGCCGTTGCAGGCTTCTCCAACTGGACCTCAAATTTCAT
TGTGGGCATGTGCTTCCAGTATGTGGAGCAACTGTGTGGTCCCTACGTCT
TCATCATCTTCACTGTGCTCCTGGTTCTGTTCTTCATCTTCACCTACTTC
AAAGTTCCTGAGACTAAAGGCCGGACCTTCGATGAGATCGCTTCCGGCTT
CCGGCAGGGGGGAGCCAGCCAAAGTGACAAGACACCCGAGGAGCTGTTCC
ATCCCCTGGGGGCTGATTCCCAAGTG.

In some embodiments, the polynucleotide sequence encoding the GLUT1 protein is a codon-optimized sequence. The polynucleotide encoding the GLUT1 protein may comprise a polynucleotide sequence at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 27)
ATGGAACCATCATCCAAAAAGCTGACCGGACGACTGATGCTTGCAGTTGG
CGGTGCGGTCTTGGGGAGCCTGCAGTTTGGGTACAATACTGGCGTAATCA
ATGCCCCGCAGAAGGTTATTGAAGAATTTTACAATCAAACGTGGGTACAT
CGCTACGGTGAATCCATTCTTCCTACAACTCTGACCACACTCTGGAGCCT
TTCTGTAGCGATTTTTTCCGTCGGGGGCATGATAGGATCATTTTCCGTCG
GTCTTTTTGTGAACCGCTTTGGCCGGAGAAATTCCATGCTGATGATGAAT
CTTCTCGCTTTCGTGAGTGCCGTCCTCATGGGATTTAGTAAACTGGGTAA
ATCTTTCGAGATGTTGATACTGGGGAGATTTATTATCGGCGTGTATTGTG
GTTTGACCACGGGCTTTGTACCAATGTATGTTGGCGAGGTTTCTCCGACA
GCATTGAGAGGTGCACTCGGGACCTTGCACCAGTTGGGCATCGTAGTAGG
AATCCTTATAGCGCAAGTTTTCGGGCTCGATTCCATCATGGGGAACAAAG
ATCTCTGGCCATTGCTCCTCTCAATAATTTTTATACCGGCATTGCTTCAG
TGTATTGTTCTTCCTTTTTGCCCAGAGTCCCCTAGGTTCCTGCTCATAAA
CAGGAATGAGGAGAATCGCGCTAAGTCCGTGTTGAAAAAACTTAGGGGAA
CTGCAGACGTTACTCACGATTTGCAAGAGATGAAGGAGGAATCTAGGCAA
ATGATGCGCGAGAAGAAGGTTACCATACTCGAACTCTTCCGCTCCCCCGC
GTACAGGCAGCCCATTCTTATCGCGGTCGTCTTGCAGTTGTCACAACAGT
TGAGTGGGATTAATGCAGTTTTCTATTATAGCACGTCCATATTTGAAAAA
GCAGGCGTCCAACAACCTGTCTATGCAACTATAGGCTCAGGCATTGTAAA
CACAGCGTTTACTGTAGTATCACTGTTTGTCGTTGAGCGGGCTGGTCGAA
GGACCTTGCACCTCATAGGACTGGCGGGCATGGCGGGCTGTGCGATTCTT
ATGACAATTGCGCTCGCGCTGTTGGAACAGCTTCCGTGGATGTCCTATCT
CTCTATAGTAGCAATATTTGGATTTGTTGCATTTTTTGAAGTTGGGCCCG
GACCTATCCCCTGGTTCATCGTCGCGGAGCTCTTTTCCCAAGGCCCAAGA
CCGGCTGCCATTGCTGTTGCAGGCTTCTCAAACTGGACGAGTAATTTCAT
AGTAGGTATGTGTTTCCAGTATGTTGAACAGCTCTGTGGGCCCTATGTCT
TTATCATCTTTACTGTGTTGCTCGTGTTGTTCTTTATCTTCACTTATTTC
AAAGTACCCGAGACAAAGGGCAGGACGTTTGACGAGATTGCATCTGGTTT
TAGACAAGGAGGTGCCTCACAGAGTGATAAAACCCCGGAGGAATTGTTTC
ATCCGCTGGGAGCCGACTCACAGGTC

Optionally, the polynucleotide sequence encoding the AAV virion genome may comprise a Kozak sequence, including but not limited to GCCACCATGG (SEQ ID NO: 28). Kozak sequence may overlap the polynucleotide sequence encoding an GLUT1 protein or a functional variant thereof. For example, the AAV virion genome may comprise a polynucleotide sequence (with Kozak underlined) at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 29)
gccaccATGGAGCCCAGCAGCAAGAAGCTGACGGGTCGCCTCATGCTGGC
CGTGGGAGGAGCAGTGCTTGGCTCCCTGCAGTTTGGCTACAACACTGGAG
TCATCAATGCCCCCCAGAAGGTGATCGAGGAGTTCTACAACCAGACATGG
GTCCACCGCTATGGGGAGAGCATCCTGCCCACCACGCTCACCACGCTCTG
GTCCCTCTCAGTGGCCATCTTTTCTGTTGGGGGCATGATTGGCTCCTTCT
CTGTGGGCCTTTTCGTTAACCGCTTTGGCCGGCGGAATTCAATGCTGATG
ATGAACCTGCTGGCCTTCGTGTCCGCCGTGCTCATGGGCTTCTCGAAACT
GGGCAAGTCCTTTGAGATGCTGATCCTGGGCCGCTTCATCATCGGTGTGT
ACTGCGGCCTGACCACAGGCTTCGTGCCCATGTATGTGGGTGAAGTGTCA
CCCACAGCCCTTCGTGGGGCCCTGGGCACCCTGCACCAGCTGGGCATCGT
CGTCGGCATCCTCATCGCCCAGGTGTTCGGCCTGGACTCCATCATGGGCA
ACAAGGACCTGTGGCCCCTGCTGCTGAGCATCATCTTCATCCCGGCCCTG
CTGCAGTGCATCGTGCTGCCCTTCTGCCCCGAGAGTCCCCGCTTCCTGCT
CATCAACCGCAACGAGGAGAACCGGGCCAAGAGTGTGCTAAAGAAGCTGC
GCGGGACAGCTGACGTGACCCATGACCTGCAGGAGATGAAGGAAGAGAGT
CGGCAGATGATGCGGGAGAAGAAGGTCACCATCCTGGAGCTGTTCCGCTC
CCCCGCCTACCGCCAGCCCATCCTCATCGCTGTGGTGCTGCAGCTGTCCC
AGCAGCTGTCTGGCATCAACGCTGTCTTCTATTACTCCACGAGCATCTTC
GAGAAGGCGGGGGTGCAGCAGCCTGTGTATGCCACCATTGGCTCCGGTAT
CGTCAACACGGCCTTCACTGTCGTGTCGCTGTTTGTGGTGGAGCGAGCAG
GCCGGCGGACCCTGCACCTCATAGGCCTCGCTGGCATGGCGGGTTGTGCC
ATACTCATGACCATCGCGCTAGCACTGCTGGAGCAGCTACCCTGGATGTC
CTATCTGAGCATCGTGGCCATCTTTGGCTTTGTGGCCTTCTTTGAAGTGG
GTCCTGGCCCCATCCCATGGTTCATCGTGGCTGAACTCTTCAGCCAGGGT
CCACGTCCAGCTGCCATTGCCGTTGCAGGCTTCTCCAACTGGACCTCAAA
TTTCATTGTGGGCATGTGCTTCCAGTATGTGGAGCAACTGTGTGGTCCCT
ACGTCTTCATCATCTTCACTGTGCTCCTGGTTCTGTTCTTCATCTTCACC
TACTTCAAAGTTCCTGAGACTAAAGGCCGGACCTTCGATGAGATCGCTTC
CGGCTTCCGGCAGGGGGGAGCCAGCCAAAGTGACAAGACACCCGAGGAGC
TGTTCCATCCCCTGGGGGCTGATTCCCAAGTG.

In some embodiments, the Kozak sequence is an alternative Kozak sequence comprising or consisting of any one of:

(SEQ ID NO: 30)
(gcc)gccRccAUGG;
AGNNAUGN;
ANNAUGG;
ACCAUGG;
and
(SEQ ID NO: 31)
GACACCAUGG.

In some embodiments, the AAV virion genome comprises no Kozak sequence. The polynucleotide sequence may be codon-optimized.

### AAV Virion Genome

The AAV virions of the disclosure comprise a vector genome. The vector genome may comprise an expression cassette (or a polynucleotide cassette for gene-editing applications not requiring expression of the polynucleotide sequence). Any suitable inverted terminal repeats (ITRs) may be used. The ITRs may be from the same serotype as the capsid or a different serotype (e.g., AAV2 ITRs may be used).

In some embodiments, the 5′ ITR comprises a polynucleotide sequence at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 32)
TTGGCCACTCCCTCTCTGCGCGCTCGCTCGCTCACTGAGGCCGCCCGGGC
AAAGCCCGGGCGTCGGGCGACCTTTGGTCGCCCGGCCTCAGTGAGCGAGC
GAGCGCGCAGAGAGGGAGTGGCCAACTCCATCACTAGGGGTTCCT

In some embodiments, the 5′ ITR comprises a polynucleotide sequence at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 6)
GCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGA
GTGGCCAACTCCATCACTAGGGGTTCCTTGTAGTTAATGATTAACCCGCC
ATGCTACTTATCTACGTA

In some embodiments, the 5′ ITR comprises a polynucleotide sequence at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 33)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACGTA

In some embodiments, the 3′ ITR comprises a polynucleotide sequence at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 34)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGAGAGGGAGTGGCCAA

In some embodiments, the 3′ ITR comprises a polynucleotide sequence at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 7)
TACGTAGATAAGTAGCATGGCGGGTTAATCATTAACTACAAGGAACCCCT
AGTGATGGAGTTGGCCACTCCCTCTCTGCGCGCTCGCTCGCTCACTGAGG
CCGGGCGACCAAAGGTCGCCCGACGCCCGGGCTTTGCCCGGGCGGCCTCA
GTGAGCGAGCGAGCGCGC

In some embodiments the AAV virion genome comprises one or more filler sequences, e.g., at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 35)
GCGGCAATTCAGTCGATAACTATAACGGTCCTAAGGTAGCGATTTAAATA
CGCGCTCTCTTAAGGTAGCCCCGGGACGCGTCAATTGACTACAAACCGAG
TATCTGCAGAGGGCCCTGCGTATG;
(SEQ ID NO: 36)
CTTCTGAGGCGGAAAGAACCAGATCCTCTCTTAAGGTAGCATCGAGATTT
AAATTAGGGATAACAGGGTAATGGCGCGGGCCGC;
or
(SEQ ID NO: 37)
GTTACCCAGGCTGGAGTGCAGTGGCACATTTCTGCTCACTGCAACCTCCT
CCTCCCTGGGTTC.

### Promoters

In some embodiments, the polynucleotide sequence encoding an GLUT1 protein or functional variant thereof is operably linked to a promoter.

The present disclosure contemplates use of various promoters. Promoters useful in embodiments of the present disclosure include, without limitation, a cytomegalovirus (CMV) promoter, phosphoglycerate kinase (PGK) promoter, or a promoter sequence comprised of the CMV enhancer and portions of the chicken beta-actin promoter and the rabbit beta-globin gene (CAG). In some cases, the promoter may be a synthetic promoter. Exemplary synthetic promoters are provided by Schlabach et al. PNAS USA. 107(6):2538-43 (2010). In some embodiments, the promoter comprises a polynucleotide sequence at least 75%, at least 80%, at least 85%, at least 90%, at least 92%, at least 93%, at least 94%, at least 95%, at least 96%, at least 97%, at least 98%, at least 99%, or 100% identical to:

(SEQ ID NO: 38)
ACTTACGGTAAATGGCCCGCCTGGCTGACCGCCCAACGACCCCCGCCCAT
TGACGTCAATAATGACGTATGTTCCCATAGTAACGCCAATAGGGACTTTC
CATTGACGTCAATGGGTGGAGTATTTACGGTAAACTGCCCACTTGGCAGT
ACATCAAGTGTATCATATGCCAAGTACGCCCCCTATTGACGTCAATGACG
GTAAATGGCCCGCCTGGCATTATGCCCAGTACATGACCTTATGGGACTTT
CCTACTTGGCAGTACATCTACGTATTAGTCATCGCTATTACCATGGTCGA
GGTGAGCCCCACGTTCTGCTTCACTCTCCCCATCTCCCCCCCCTCCCCAC
CCCCAATTTTGTATTTATTTATTTTTTAATTATTTTGTGCAGCGATGGGG
GCGGGGGGGGGGGGGGCGCGCGCCAGGCGGGGCGGGGCGGGGCGAGGGGC
GGGGCGGGGCGAGGCGGAGAGGTGCGGCGGCAGCCAATCAGAGCGGCGCG
CTCCGAAAGTTTCCTTTTATGGCGAGGCGGCGGCGGCGGCGGCCCTATAA
AAAGCGAAGCGCGCGGCGGGCGG

In some embodiments, a polynucleotide sequence encoding an GLUT1 protein or functional variant thereof is operatively linked to an inducible promoter. An inducible promoter may be configured to cause the polynucleotide sequence to be transcriptionally expressed or not transcriptionally expressed in response to addition or accumulation of an agent or in response to removal, degradation, or dilution of an agent. The agent may be a drug. The agent may be tetracycline or one of its derivatives, including, without limitation, doxycycline. In some cases, the inducible promoter is a tet-on promoter, a tet-off promoter, a chemically-regulated promoter, a physically-regulated promoter (i.e., a promoter that responds to presence or absence of light or to low or high temperature). Inducible promoters include heavy metal ion inducible promoters (such as the mouse mammary tumor virus (mMTV) promoter or various growth hormone promoters), and the promoters from T7 phage which are active in the presence of T7 RNA polymerase. This list of inducible promoters is non-limiting.

In some cases, the promoter is a tissue-specific promoter, such as a promoter capable of driving expression in an endothelial cell to a greater extent than in a non-endothelial cell. In some embodiments, tissue-specific promoter is an endothelial-specific promoter. In some embodiments, tissue-specific promoter is a selected from any various endothelial-specific promoters including but not limited to FLT1 (Vascular Endothelial Growth Factor Receptor 1), Tie1 (Tyrosine Kinase With Immunoglobulin Like And EGF Like Domains 1), VE-Cadherin (Vascular Endothelial Cadherin), hSYN1 (human synapsin), INA (alpha-internexin), NES (nestin), TH (tyrosine hydroxylase), FOXA2 (Forkhead box A2), CaMIKII (calmodulin-dependent protein kinase II), and NSE (neuron-specific enolase). In some cases, the promoter is a ubiquitous promoter. A “ubiquitous promoter” refers to a promoter that is not tissue-specific under experimental or clinical conditions. In some cases, the ubiquitous promoter is any one of CMV, CAG, UBC, PGK, EF1-alpha, GAPDH, SV40, HIBV, chicken beta-actin, and human beta-actin promoters.

In some embodiments, the promoter sequence is selected from Table 3. In some embodiments, the promoter comprises a polynucleotide sequence at least 75% at least 80%, at least 85%, at least 90%, at least 92%, at least 93%, at least 94%, at least 95%, at least 96%, at least 97% at least 98%, at least 99% or 100% identical to any one of SEQ TD NOS 1-3 and 39-51.

TABLE 3

| | | SEQ |
|---|---|---|
| | | ID |
| PROMOTER | SEQUENCE | NO: |
| Human FLT-1 | TTTGCTTCTAGGAAGCAGAAGACTGAGGAAATGACTTGGGCGGGTG | 1 |
| (hFLT-1) | CATCAATGCGGCCAAAAAAGACACGGACACGCTCCCCTGGGACCT | |
| | GAGCTGGTTCGCAGTCTTCCCAAAGGTGCCAAGCAAGCGTCAGTTC | |
| | CCCTCAGGCGCTCCAGGTTCAGTGCCTTGTGCCGAGGGTCTCCGGT | |
| | GCCTTCCTAGACTTCTCGGGACAGTCTGAAGGGGTCAGGAGCGGCG | |
| | GGACAGCGCGGGAAGAGCAGGCAAGGGGAGACAGCCGGACTGCG | |
| | CCTCAGTCCTCCGTGCCAAGAACACCGTCGCGGAGGCGCGGCCAGC | |
| | TTCCCTTGGATCGGACTTTCCGCCCCTAGGGCCAGGCGGCGGAGCT | |
| | TCAGCCTTGTCCCTTCCCCAGTTTCGGGCGGCCCCCAGAGCTGAGT | |
| | AAGCCGGGTGGAGGGAGTCTGCAAGGATTTCCTGAGCGCGATGGG | |
| | CAGGAGGAGGGGCAAGGGCAAGAGGGCGCGGAGCAAAGACCCTG | |
| | AACCTGCCGGGGCCGCGCTCCCGGGCCCGCGTCGCCAGCACCTCCC | |
| | CACGCGCGCTCGGCCCCGGGCCACCCGCCCTCGTCGGCCCCCGCCC | |
| | CTCTCCGTAGCCGCAGGGAAGCGAGCCTGGGAGGAAGAAGAGGGT | |
| | AGGTGGGGAGGCGGATGAGGGGTGGGGGACCCCTTGACGTCACCA | |
| | GAAGGAGGTGCCGGGGTAGGAAGTGGGCTGGGGAAAGGTTATAAA | |
| | TCGCCCCCGCCCTCGGCTGCTCTTCATCGAGGTCCGCGGGAGGCTC | |
| | GGAGCGCGCCAGGCGGACACTCCTCTCGGCTCCTCCCCGGCAGCGG | |
| | CGGCGGCTCGGAGCGGGCTCCGGGGCTCGGGTGCAGCGGCCAGCG | |
| | GGCGCCTGGCGGCGAGGATTACCCGGGGAAGTGGTTGTCTCCTGGC | |
| | TGGAGCCGCGAGACGGGCGCTCAGGGCGCGGGGCCGGCGGCGGCG | |
| | AACAAGAGGACGGACTCTGGCGGCCGGGTCGTTGGCCGCGGGGAG | |
| | CGCGGGCACCGGGCGAGCAGGCCGCGTCGCGCTCACC | |
| Human FLK-1 | TGGAGCCGCCAAATATTTTGGGAAATAGCGGGAATGTTGGCGAACT | 85 |
| (hFLK-1) | GGGCAAGTGCGTTTTCTGATTAAGAGCAACCAGATTCAGCTTTTTA | |
| | AACTACAATTATACTGGCCAAACAAAATACCCTTATACAAAAACCA | |
| | AAACTACTGGCAGGAGTCGCTGCCAGCTTGCGACCCGGCATACTTG | |
| | GCTGAGTATCCGCTTCTCCCTTGTGGCTCCAAACTGCTGCAGATTCT | |
| | CGGCCACTTCAGACGCGCGCGATGGCGAAGAGGGTCCTGCACTTTG | |
| | ACGCGCCTGGTGAGGGAGCGCTGCTCTTCGCAGCGCTCCTGGTGAT | |
| | GCTCCCCAAATTTCGGGGACCGGCAAGCGATTAAATCTTGGAGTTG | |
| | CTCAGCGCCCGTTACCGAGTACTTTTTATTTACACCAGAAACAAAG | |
| | TTGTTGCTCTGGGATGTTCTCTCCTGGGCGACTTGGGGCCCAGCGCA | |
| | GTCCAGTTGTGTGGGGAAATGGGGAGATGTAAATGGGCTTGGGGA | |
| | GCTGGAGATCGCCGCCGGGTACCCGGGTGAGGGGGGGGCTGGCC | |
| | GCACGGGAGAGCCCCTCCTCCGCTCCGGCCCCGCCCCGCATGGCCC | |
| | CGCCTCCGCGCTCTAGAGTTTCGGCACCAGCTCCCACCCTGCACTG | |
| | AGTCCCGGGACCCCGGGAGAGCGGTCAATGTGTGGTCGCTGCGTTT | |
| | CCTCTGCCTGCGCCGGGCATCACTTGCGCGCCGCAGAAAGTCCGTC | |
| | TGGCAGCCTGGATATCCTCTCCTACCGGCACCCGCAGACGCCCCTG | |
| | CAGCCGCGGTCGGCGCCCGGGCTCCCTAGCCCTGTGCGCTCAACTG | |
| | TCCTGCGCTGCGGGGTGCCGCGAGTTCCACCTCCGCGCCTCCTTCTC | |
| | TAGACAGGCGCTGGGAGAAAGAACCGGCTCCCGAGTTCTGGGCAT | |
| | TTCGCCCGGCTCGAGGTGCA | |
| Human Tie-1 | AGCTCCTCCCAGCCTCAGGCCCAGGAATGGGAATCTCTGTGGGTCA | 2 |
| (hTIE-1) | CACATCAGTAGGGAGGTCTTTCCCGATCCTTTTCTATGCTACTCCAG | |
| | GAGTCAAAGCGTCTCCTGGGACTTTTCAGGGCGCTTCAGAAGAGCC | |
| | CTGGGCCTAAACCAGCTCAACCAAGCTGCAGGGACCCAGCCTCCTG | |
| | AGAAAAGTGAATGTGAGCCCGGTGCATTCAGAGGAGAATGAAGCC | |
| | TTCACCCAGAACACACTCTGGGAAGATGTCCCAGGCCCAGGGGGA | |
| | GGGTTTGTACTACCAGACCTAAGTCACCTAAACTGACACCAAGTCT | |
| | CATCCATCCCAACCATTCCATTCCGGGTCAGAGGGGTCATCGATTT | |
| | AACCAGCAAGGCTGCCCATCCAACGGTTGCTCCCTCTGCTCCCTGG | |
| | AAGGGCCTCCTCGTGGGCGTTCTGTACCTACAGGTCTTGTTCCGTTC | |
| | TGGGAACTGCCAGTGGTGGCAAGAGGTGGAGCAACGGGTGCCAGG | |
| | GCAGGGAGAGGTGAGTCTGGGAGGGAAGCAGAGGCAAGATCCATG | |
| | GGGCTTTAGAGACTTTGCCAAAGCAGTGCGACTGCTCCCAGGTTGT | |
| | TGTCAGCCGTCAAGAGTGAGTGCACCTCCCTGGGCAGACTTCTGCT | |
| | GCCCCAGTGCCCAGGAATAGGCAGGGGTTTGCCGCAAAATGAATG | |
| | ACACCTGGCAGACAATAAGCTGAAGCTTTCATTAGCAGCTTAAGCT | |
| | GAGGACTATCTATGCAACCGATACTCCCTGTGTGCTCCCCGGGACT | |
| | GCTTAATGTGAGCCCTTGTGGAGCGATTGGCACCAAGAAAGCAAG | |
| | GACTAAGTCAGAAGTTCAAGTCCCAGCCTTGCCACAGCCTCAGGGT | |
| | GCCCTCGAGCACAGCAAGCCTCAGTTTTCCCATCTGTACAATGAGA | |
| | GAGGTACACAAGGTAGACTCGAAGGCTCTTTGTTGCCAGGGCCCTG | |
| | TGTTCCTTTGAGTGTATGTGCTTCTCAGGCCCACAGAGGTCCTTTGT | |
| | GTTTCGTATGTGAACTGCTCTCTAGGAAACCCATGTAACTGTCTGTG | |
| | TCCTGGGGCACATACATGAGGACTCATGTGGGCCGTATTGTGTGTT | |
| | TGTGCCGGGGGGAGGGGAGACCCCAGAACAATGTCCCCCACCCCA | |
| | CCCCCCTCCTCAATAGGCGGAAGCCACTGGCTTCCTCCCTTTCCTGC | |
| | CTCCTGCCTCCTTTGTGCCAGCAAGACTGAGTACTGGAGAGAGACA | |
| | GGGGATGGGAAAAATCAGTCCAGCTGTCCCCAGGTCTGCCCTTACC | |
| | ATAACCTTCCCCCCACCTCAAGTGACTCCTCCCAGGCCACACCCAT | |
| | CCCCAGCCTTGTGGGGGCCAGATTGGGGGGCCTAGAGGCTCAAAG | |
| | GCAGAATGAGTCCTCCCACCCCCTACCCTGCCACCCCTCCCACCCA | |
| | AGCCACCTCATTTCCTCTTCCTCCCCAGCACCGACCCACACTGACCA | |
| | ACACAGGCTGAGCAGTCAGGCCCACAGCATCTGACCCCAGGCCCA | |
| | GCTCGTCCTGGCTGGCCTGGGTCGGCCTCTGGAGT | |
| Mouse Tie-1 | AAGCTTCCGACCGTTAGTCAGAGAACTGTAAGTGCTCAGAGCCTGG | 86 |
| (mTIE-1) | CTGACAATGATCTGGAATGAACCAGATAACAACATAATAAAATCTC | |
| | AGTAAAATAATTTAACAGTTAGCTTGGAAGCTGGTCAGCTCTGGGG | |
| | AAATCAGGGTAAATTGTGCTGTCATGAACTGTCCCACACTGACATC | |
| | GGCCAAAGTGAATATGAACTTTGGTAGATCCAATGCCTGTTCTATT | |
| | TATTTTTCCAGTGAAAAGTATTTTGATAGAGCTTTTCATTTTGTAAA | |
| | TACACTGAGTTAACCAAAATATCATGGATTTCCGTTTGTTCTTAAGA | |
| | CATGCAACTCGTCTACGGCTATACCACTCTGAACGCGCCCGATCTC | |
| | GGAAGACATGCAACTCAAATGTAAATACAGTAGAATATTACTTAGG | |
| | TAGAAACTCCTGGTGATTTTAAAAGATTGGAAAAGAATATGAGGA | |
| | AGAGTTGAATAATGCAAATTCTAGTGTGTGTGCTACCGAAGTGAAC | |
| | ACTTAATGCACAGTCTACAGACTAGGACATTTTATCGTGTGTTGTA | |
| | AAATTGGGTAGAAACTTGTGTTTGTGAAAACTGAGCATTAAAACCT | |
| | TACAGAGACCGTTTCTTGTTTACTTTTGAAAAAAAAAAGAGTCACG | |
| | TGAGCCTCATTTTGTATTTGTGTGTGTGTGTGTGTGTGTGTCTCCCCT | |
| | CCTCCCAGCGTGTGTGTGCTGGGAGGAGGGGAGACCCCAGAACAA | |
| | TGTCCTGCCTCCAAACCTTCTCAATAGGCGGAAGCCACTGGCTTCCT | |
| | CCCTTTCCTGTCTCCCGTGCTCCAGCAATGCAGATGGAAGGGACCG | |
| | AAGGGATGGGAGAGAGAGCCCAACCATCCCCAGATCTGTCCTTGTC | |
| | ACAACCTGCCTCCCACCTCTAATGCCCCCCCTTCCAGAGACTTCCAG | |
| | GCCACACCCATCCCGGGCTTGTGGGGGCTGGACACGGGAGGACTA | |
| | CAGGCGACAACTCTTCCCACCCTCTCTCCCTGCCACCCCTCCTACCC | |
| | TAACCATCATTTCCTCTTCCTCCCCAGCACCGAGGTGCACTGAGCTG | |
| | GACAGGCTGAACACTCAGACCCACAGCAACTGACCCCGGGCCCAG | |
| | CTGGCCTTGGCTGGCCCAGGGCAGCTTCCAGAGT | |
| Human Tie-2 | GCTGGAGTGCAGTGGCACGATCTCGGCTCACTGCAACCTCTGCCTC | |
| (hTIE-2) | CCAGGTTCAAACAATTCTCCTGCCTCAGCCTCCAGAGTAGCTGGGG | |
| | TTACAGGTGCACGCCAGCAAGCACAGCTAAATTTTGTATTTTTAGT | |
| | AGAGATGGGGTTTTGCCATGTTGGCCAGGCTGGTCTCAAACTCCTG | |
| | ACCTCAGGTGATCCACTCCCAAAGTGCTGGGATTATAGGCGTGAGC | |
| | CACTGTGCCAGGCCCACTGTTTTTGTTTTTTTTTTTCGTGATGACAA | |
| | ATTTAAAGTCATCTCATAGGAATAGAAAATAGCTTTTTAGTAGAAG | |
| | CTCTTGGAATTTAAATTGAGACTGAATGGAAAGATGAAAGAAAAT | |
| | AAACTTATTAACATTTAATGAGAACCTTCAAAGAACTAGGCATAGT | |
| | ACCAAATGGTTTTATATTTTTAAACCTCATTTATTCCTCTCAAAACA | |
| | CCTGGGAAGGAGATATTTTTGCCATTTCACAGCTGTTGAAACTGAG | |
| | GCTCAAAAAGACTAAGTAACTTTTCTCAGCTACACATGTGGCTGAG | |
| | CCAGTATTTGAACCCAGTTCTGTTTGCAGACAGAACCTGGGCTTTTT | |
| | CACACCTGCAAACTGGAAACATTAATTGGTTCTTAAGATCATCATC | |
| | GATGTGATAAAACCTGGGACAGAAATTAGTCAAGACTAGCTGCATC | |
| | TGCCTTTTCCTCTGGTGGGTAGGAAAAGGAGGAGTATAATGATTTC | |
| | CTCAGGCATGAAGGTCGATGATGAGCAAAGTGTATACTCTCTAATC | |
| | TAATGTCATAATTCATATTGTGGAGTAATTATCTGGATAAGTGTAG | |
| | GGTCTCTGACCTCATTCTAGATATTGTACATTCCATGGCTATTTTCA | |
| | TTTTGGTCCATGAACTCTCTTTGCTCTCATGAGCACCATTTTTATCCC | |
| | AATCTAATCCTGTATGTTTGTGTTTTTACACAGATTAGTTTTTAAAT | |
| | GTTATATATAATTTGCTTCTGAAACACCATTGCTCAATGACTACCAA | |
| | ATCTTTCTCATTACCAAAATCCTTCTATGCCAACTTCTTCAAGAAAT | |
| | TTGATCACCTTTAGATGAATTGTTAATGAAAATTAAAGCTATAGCC | |
| | GGCAACATGGGTATCTTTGGGCTAATGGCCAACCAACAGGCCATCT | |
| | GTGTGAAAGAAAACAGGCTAACAATTTTGGACTCTGGTCTCTTGGG | |
| | GCTACATTGAGCATTGACCTCACCGGTGCTCACTGAAATTAATTGC | |
| | TTTTCAGGTTGTATTTTCTCATCACGGAAACCTTCTTCTCCCAATTC | |
| | AAACCATGTGGGTTAAAATGAGAAAACAAAAGCCAAAACGGCTTC | |
| | CCACACCCAAAAGCTCCTTCTGTCAGAGATCCCAGTAGCCCCGGGA | |
| | GAGCTGTTAGAAGTCTGAGAAGGATTGGTCATCATCGCATACCATA | |
| | CATAGGTGGAGGGCTTGTTATTCTCAGTTTCCCGCCTATGAGAGGA | |
| | TACCCCTATTGTTTCTGAAAATGCTGACCGGGACCCACACTTCCAA | |
| | CAAAAATTCCTCTGCCCCTACAGCAGCAGCAAAAGCAGCAGCAGA | |
| | AGCAACAGCAACAGATAAGTGTTTTGATGAATTGCGAGATGGATA | |
| | GGGCTTGAGTGCCCCCAGCCCTGCTGATACCAAATGCCTTTAAGAT | |
| | ACAGCCTTTCCCATCCTAATCTACAAAGGAAACAGGAAAAAGGAA | |
| | CTTAAAACTCCCTGTGCTCAGACAGAAATGAGACTGTTACAGCCTG | |
| | CTTCTGTGCTGTTCCTTCTTGCCTCTAACTTGTAAACAAGACGTAGT | |
| | AGGACGATGCTAATGGAAAGTCACAAACCGCTGGGTTTTTGAAAG | |
| | GATCCTTGGGACCTCATGCACATTTGTGGAAACTGGATGGAGAGAT | |
| | TTGGGGAAGCATGGACTCTTTAGCCAGCTTAGTTCTCTGTGGAGTC | |
| | AGCTTGCTCCTTTCTGGTAAGGTTTGGCTTTATTTTTTTTAATTTAGT | |
| | ATTTTAAAAAACAGAGTTAGTGATTTCTGGGTGCTCTCCCCAAATCT | |
| | CATCAGTGCTGATGAACAAGGGGTGGCTGTAGCAAAGGCACCATTT | |
| | C | |
| Human VE- | CTAGTAGCAGAAACAAGGTCCTCTGGAAGAGCAACTGATGCTCTTA | 3 |
| cadherin | GGTACTGAAGCATCATCCTGCCCCAGAGACCACTCGCATATGAAGC | |
| (hVE- | ACACATATTCAGTCTGCCTTACTTGTGTTAATGATTGCCAGTGTCCC | |
| caderin) | TCTGACCTCCTAGCCCTGAAAAGTGTGGCCTGAAGGTCATTTCAGA | |
| | GACGGGGAGAGCTGCTCAGAGAAGCCAATCGGCGAGTCTAGGACA | |
| | CACAGACAGGATCTAGTCCCAGAGTTCGCTAGCCTAGGTGAGCGTC | |
| | CCCTGGCCCCTTATACCACTTCCTTCTCCAGCTTGCATCTAATCTGC | |
| | TCTGGCAGACCATCGTGTTTCCTGTCTTCCTGGCAGCCTCCAGCACG | |
| | CTCAGTGCTACTCCCTGCGCATGCGCCCTCCTCCCAGTACCTTCTCT | |
| | GACTCCAGTGGGCTTGGAGTGCGAGGAGGAAGGGTGAGGAAGGGG | |
| | TGAAATCAGGTATTGGATCCACAGGGGGTCTGAAGAGCACTAGCCT | |
| | GGCCTTTTGGGACTGAACTTCTGCTATGAAGACCTCCACTGCCATCC | |
| | CTGGAGTCCGGGGCACATCCAAGGCTTGCTGTCCATCGTTTACTGTT | |
| | TACAGATGACAACAATGACTGTGTTCGGGGCAGAAATATCCACCAG | |
| | GGCTAGAGTACAAAAGGAGTTTGCATTGATGGCCGGACAGGCCCT | |
| | GTCCCTGGCAGCCTGCCAGCGCTGAGTATGAGACCCAGCGGGAAGT | |
| | GCTACCCTGGCAGACGTGTCCACTGAGTACACAGACCACCAAGGCA | |
| | GGCAGCTCTCGGGGAAGCTGTCTATGCTGGGCCAGCCCACCTTGAG | |
| | GGCAGGGAACAGAACAGATTGTGGCAGAGAGGAAAATGTGGAGCT | |
| | TCTGTTTGTTCACAGACACACGCACTCGCCCACGCACGCACGCACG | |
| | CACGCACGCACGCACGAATGCACGCACGCAGTAGTTGAATGCTATG | |
| | GATTCCGCTCAGAGCTGAGAACAGCCCCAGCGACAGTTCCCTGGCC | |
| | TCTCTCCTTACTCTGATGTCCTCATCTGTCTTCACATGGTCTCAGGA | |
| | CGCTAATACTCCATCCTAATGTACACTCCTTTCCCTGGGCCTCCGTT | |
| | CCAGTTCAGTTCTCAGAGGACCTGGAGGGAGTGATTGGCTACACCA | |
| | ACTTTGCTTTCGTTCACCAAGCCCATGTCTCTACTTGGGTGTCTAAT | |
| | GGGCATCTCCAACATTACCTACCCCAAACAGAAAACCCTTTCTTCC | |
| | CCCCAACCACACCCCACCCTACCCCCACAGTATTTTCTCCATGCCCG | |
| | GAAAGATCTGCTCTCTTATGGTCCCTCTTTGCCTCACTGAAAAGCAG | |
| | GACAAGTTGGGGACTTCCCAAACTTTTATGCATGAAGAAACCCAGG | |
| | CAATTTGCCAAAAGGTACACTCTGGGGGTCTGTCATTTACTCTGAG | |
| | CCAGAACCCTGAAATTTTTACTAACCCATCACATAATGAATGAAGA | |
| | GAATCTTTTTCTTTTTTTTTTTTTTTCTTTTTTTTTGGTTTTTCGAGAC | |
| | AGGGTTTCTCTGTATAGCCCTGGCTATCCTGGAACACACTCTGTAG | |
| | ACCAGGCTGGCCTCGAACTCAGAAATCCACCTGCCTCTGCCTCCCG | |
| | AGTGCTGGGATTAAAGGCGTGCGCCACCACGCCTGGCTGAATGAA | |
| | GAGAATCTTGACCTCATCTCCCCAGCCTCTTGGTCCTGAGGGACCCT | |
| | GGTCTACCTACTGCTTTGCTGTCTTCTTAGCTCTTCTTACTTTTTTGC | |
| | TGACTCAGACCTATGGCTATCTCCATTATACAGATGAGGAGACTGA | |
| | GGCATGGATCCCTGGTTGGTCCATGGTCACGTGAAGCCCATCACCC | |
| | AGTATTTGTAAAGTGAGATGGGCCAGGCTGGTACCTTGGAACTGAA | |
| | ACTCACACTGCCCTACCTGGAAGAATCTGACAGGCAAAATCTGCTG | |
| | CTGAAAGTGATTGTCTGTCACGTTTCTCAGCTGCCCGACTCTGAGA | |
| | ACTCCACAGCCCCCTTTCGTTCCACCATACTACAGAGTCGCCACGG | |
| | AAAGCCGGCTCTGTGGAGAAGCTGAGGTAGCTGGGTTTCTGTCTGG | |
| | GTTACTCTGTCCAGCGAGGAAACAAGTACCTTAGACCCACTAAGCC | |
| | TCTGCTTTCTGAACTGTAAAGTGGGGGATATGACACCTGCCTCCCA | |
| | GGGATGGCTGAATGCTCTGGCAGAAGCTTAGAGCCCCCACAGCTAC | |
| | CCCTAGGCTCACAGCTCCTCCGATGAGACCTAGAATTGAGGTATGA | |
| | GTTGAATACCCCAGGCAGGTCCAAGGCTTCCACGGGCCCAGGCTGA | |
| | CCAAGCTGAGGCCGCCCACCGTAGGGCTTGCCTATCTGCAGGCAGC | |
| | TCACAAAGGAACAATAACAGGAAACCATCCCGAGGGGAAGTGGGC | |
| | CAGGGCCAGTTGGAAAACCTGCCTCCCTCCCAGCCTGGGTGTGGCT | |
| | CCCCTCTCCCCTCCTGAGGCAATCAACTGTGCTCTCCACAAAGCTCG | |
| | GCCCTGGACAGACT | |
| Human VE- | CATCCATGCCCATGGCCTCAGATGCCAGCCATAAGCTGTTGGGTTC | 88 |
| cadherin | CAAACCTCGACTCCAGGCTGGACTCACCCCTGTCTCCCCCACCAGC | |
| (hVE- | CTGACACCTCCACCTGGGTATCTAACGAGCATCTCAAACTCAACCT | |
| caderin) | GCCTGAGACAGAGGAATCACTATCCCCTCCTCCTCCAAAAATATCC | |
| (short | TTCCATCACACTCCCCATCTTGTGCTCTGATTTACTAAACGGCCCTG | |
| version) | GGCCCTCTCTTTCTCAGGGTCTCTGCTTGCCCAGCTATATAATAAAA | |
| | CAAGTTTGGGACTTCCCAACCATTCACCCATGGAAAAACAGAAGCA | |
| | ACTCTTCAAAGGACAGATTCCCAGGATCTGCCCTGGGAGATTCCAA | |
| | ATCAGTTGATCTGGGGTGAGCCCAGTCCTCTGTAGTTTTTAGAAGCT | |
| | CCTCCTATGTCTCTCCTGGTCAGCAGAATCTTGGCCCCTCCCTTCCC | |
| | CCCAGCCTCTTGGTTCTTCTGGGCTCTGATCCAGCCTCAGCGTCACT | |
| | GTCTTCCACGCCCCTCTTTGATTCTCGTTTATGTCAAAAGCCTTGTG | |
| | AGGATGAGGCTGTGATTATCCCCATTTTACAGATGAGGAAACTGTG | |
| | GCTCCAGGATGACACAACTGGCCAGAGGTCACATCAGAAGCAGAG | |
| | CTGGGTCACTTGACTCCACCCAATATCCCTAAATGCAAACATCCCC | |
| | TACAGACCGAGGCTGGCACCTTAGAGCTGGAGTCCATGCCCGCTCT | |
| | GACCAGGAGAAGCCAACCTGGTCCTCCAGAGCCAAGAGCTTCTGTC | |
| | CCTTTCCCATCTCCTGAAGCCTCCCTGTCACCTTTAAAGTCCATTCC | |
| | CACAAAGACATCATGGGATCACCACAGAAAATCAAGCTCTGGGGC | |
| | TAGGCTGACCCCAGCTAGATTTTTGGCTCTTTTATACCCCAGCTGGG | |
| | TGGACAAGCACCTTAAACCCGCTGAGCCTCAGCTTCCCGGGCTATA | |
| | AAATGGGGGTGATGACACCTGCCTGTAGCATTCCAAGGAGGGTTAA | |
| | ATGTGATGCTGCAGCCAAGGGTCCCCACAGCCAGGCTCTTTGCAGG | |
| | TGCTGGGTTCAGAGTCCCAGAGCTGAGGCCGGGAGTAGGGGTTCA | |
| | AGTGGGGTGCCCCAGGCAGGGTCCAGTGCCAGCCCTCTGTGGAGAC | |
| | AGCCATCCGGGGCCGAGGCAGCCGCCCACCGCAGGGCCTGCCTATC | |
| | TGCAGCCAGCCCAGCCCTCACAAAGGAACAATAACAGGAAACCAT | |
| | CCCAGGGGGAAGTGGGCCAGGGCCAGCTGGAAAACCTGAAGGGGA | |
| | GGCAGCCAGGCCTCCCTCGCCAGCGGGGTGTGGCTCCCCTCCAAAG | |
| | ACGGTCGGCTGACAGGCTCCACAGAGCTCCACTCACGCTCAGCCCT | |
| | GGACGGACAGGCAGTCCAACGGAACAGAAACATCCCTCAGCCCAC | |
| | AGGCACGGTGAGTGGGGGCTCCCACACTCCCCTCCACCCCAAACCC | |
| | GCCACCCTGCGCCCAAGATGGGAGGGTCCTCAGCTTCCCCATCTGT | |
| | AGAATGGGCATCGTCCCACTCCCATGACAGAGAGGCTCC | |
| Human | GTCTCCCAGGCATGACTCCAACAATGCATCCCATGGGATTTGGGGT | 89 |
| Intercellular | TCCCCAGATCTGGGGCTTGTAGGCCTGACTCTCCCCTGTGCACACGT | |
| Adhesion | CTCATACACGCATGCGTGCACCCATTGCCTGCCCCGCCCCTTGCAC | |
| Molecule 2 | AGGGAGTCAGCAGGGAGGACTGGGTTATGCCCTGCTTATCAGCAGC | |
| (hICAM-2) | TTCCCAGCTTCCTCTGCCTGGATTCTTAGAGGCCTGGGGTCCTAGAA | |
| | CGAGCTGGTGCACGTGGCTTCCCAAAGATCTCTCAGATAATGAGAG | |
| | GAAATGCAGTCATCAGTTTGCAGAAGGCTAGGGATTCTGGGCCATA | |
| | GCTCAGACCTGCGCCCACCATCTCCCTCCAGGCAGCCCTTGGCTGG | |
| | TCCCTGCGAGCCCGTGGAGACTGCCAGTC | |
| Human Von | ATCTTTAGCCGATCCATTCAACCCTGGCCAGGATCCAAATGGACTG | 90 |
| Willebrand | TTTTTGTCAGGGCCAGGACCGGATCCTTCATACCTGGGGTGCATAG | |
| Factor (hVWF) | GAAGTGTTAGTACTCCCCTTCCTCCAAACACAGCAGCAAAATTGGC | |
| | TCAGGTTGAGGTGTTTTTCTCAACTTCCCTGGAGTCCAGCCCTGGAA | |
| | GCTGGATCAGGAAGCTGTGTTGTTCTACTGTGATTCCCCCTGGCCTG | |
| | TATCAGCTTGCCCTGAAACAACCAGCATTCCTGGTTATCCCACACA | |
| | GGTGGGGCACTCTAGGAAGACCAGGGATCAAGTGTGGGGGTGTAG | |
| | GGATAGGGGGTGTTTGGGGAGGGCAAGGCAGTTAATTAAGGCAGC | |
| | TGCCAGGAGGTCTCCCTCCAAACTCTACAAAGCTTTATCAGCTTGG | |
| | AGGTACTTCTAATACCATTTCCTTTCATTGTTTCCTTTTGGTAATTAA | |
| | AAGGAGGCCAATCCCCTGTTGTGGCAGCTCACAGCTATTGTGGTGG | |
| | GAAAGGGAGGGTGGTTGGTGGATGTCACAGCTTGGGCTTTATCTCC | |
| | CCCAGCAGTGGGGACTCCACAGCCCCTGGGCTACATAACAGCAAG | |
| | ACAGTCCGGAGCTGTAGCAGACCTGATTGAGCCTTTGCAGCAGCTG | |
| | AGAGCATGGCCTAGGGTGGGCGGCACCATTGTCCAGCAGCTGAGTT | |
| | TCCCAGGGACCTTGGAGATAGCCGCAGCCCTCATTTGCAGGGGA | |
| Ple261 | TGGCTTCCGGAGGGTGGCCTGGGGGCTGGGGTGCCAGGGACACCA | 91 |
| (Human | TCGCCACTGGTGGGAGGGCAGGGCACAGCCCCTCCGTGTCCCTTTG | |
| Claudin5) | TCTCTCCTGTCTGAAGGCCAGAGCAGGCTGCTAGGCCTGGGGCCAC | |
| | CACTGCCCCTGGGTGCTACACCCAGTGTGCTGGGTCACTGGGAACT | |
| | TCCTGAAGTGGTGTCACCTGAACTGGGCCCCCAAGGATGGGGTGCG | |
| | GGCAGTACCGCAGGAAGAGGAGCAGCCCCTGTGAAGATTGAGAGG | |
| | TCTGGGAAGCCCCTGCGGCTTGGGAGAGTGGGGGTCGCCAGGCAG | |
| | GGGGAAAGCCCCTGTGCCACCGCTTTTTGCCAGAGACTCAGGCTCC | |
| | AGAGAGGCAGTGAGTGGCATGGGGGGTGAGGCTGGGGCCCTGGGC | |
| | CTGACCTCCACACGCCTGCCTGGCCTCTCTGTTTGCCATGGGATGAG | |
| | AGAGACAGTGCTGGGACTCAGAGCGGGGCTGGAGAGTGAGAGTGC | |
| | GAGAAAGGGCCTGGGTGGGGCTTGGACCCCGGGGCGGGCTTTCTG | |
| | GAGAGCCCCCCTACGAGGGCCTCTACGGCGGTGACGGGGTGGGGG | |
| | GCTTCTGCAAACCTTGGTCAGGGAAGTGGAGCTGGCTCGAGTGGAA | |
| | GAGACCACCCGGCTCAGTCGGGGATGTGGGAGTGGACTGGGTGGT | |
| | GCAGACTGGGGGTCGAGCGCCTTCTGAAGTGACGGGGCCGGGACG | |
| | CGCAGGGAGGCGGCCCAAGAAGCGCGCCCTAGGCCAGCCCAGAAT | |
| | GCGCTCGGCCGCGACTAGGACAACGGCGGGTGGGGCTGGGGGCGG | |
| | CTGCCGGGCGGGGAGCGGTCCCGCGCCCTCAGCTACCCCTCAAGAG | |
| | CCGTTGTTTCCCTAACTTCAGCTGCCAGAGGCTCTGTGATTGGCTGC | |
| | GGCACGATGACCCGCGCACGGATTGGCTGCTTCGGGCCGGGGGGC | |
| | CGGGCCCGGGGGACAGAATCCGCCCCCGAACCTTCAAAGAGGGTA | |
| | CCCCCCGGCAGGAGCTGGCAGACCCAGGAGGTGCGACAGACCCGC | |
| | GGGGCAAACGGACTGGGGCCAAGAGCCGGGAGCGCGGGCGCAAA | |
| | GGCACCAGGGCCCGCCCAGGGCGCCGCGCAGCACGGCCTTGG | |
| Human | CGCCTTGCTGTGCCACTTTGGGACTTCCCTCCCTAGCCTGAGCTTCA | 92 |
| Endoglin | GTTTTCCTGCCTGTTAGGCAGCCCCATGTCAACTGCACTTAGTAGGC | |
| (hENG) | CGGGTTTGATGCCCGACAAGACGTGAAGTGGTGGAGGTGGGCAGG | |
| | ATCCCAGCGCTACCATCTTCTTGAACCAGTGATCTCAACACATCGG | |
| | ATTTCTGTTTCCTCATCTGCAAAATGGGATCAGTGAGCTCAGGTGG | |
| | GTCACAAATTCTACAGGAACTACTTTAGCCAAGCCCGGCCCCCTGA | |
| | AAGTTCCCCTCGGTGGGCTGTTAGGGTGATTGTTTTCATCTGTGGGG | |
| | CTCCCTGATGCGTCCCACCCACCAGCCTTGGAGAGGGTGGGATGGG | |
| | AGGGTGGGGTGCTTGGGGAGACAAGCCTAGAGCCTGGGCCCTCCC | |
| | ACCCCACTGCCTCCCCCCATCCCAGGGCCCCCCACCCAGTGACAAA | |
| | GCCCGTGGCACTTCCTCTACCCGGTTGGCAGGCGGCCTGGCCCAGC | |
| | CCCTTCTCTAAGGAAGCGCATTTCCTGCCTCCCTGGGCCGGCCGGG | |
| | CTGGATGAGCCGGGAGCTCCCTGCTGCCGGTCATACCACAGCCTTC | |
| | ATCTGCGCCCTGGGGCCAGGACTGCTGCTGTCACTGCCATCCATTG | |
| | GAGCCCAGCACCCCCTCCCCGCCCATCCTTCGGACAGCAACTCCAG | |
| | CCCAGCCCCGCGTCCCTGTGTCCACTTCTCCTGACCCCTCGGCCGCC | |
| | ACCCCAGAAGGCTGGAGCAGGGACGCCGTCGCTCCGGCCGCCTGCT | |
| | CCCCTCGGGTCCCCGTGCGAGCCCACGCCGGCCCCGGTGCCCGCCC | |
| | GCAGCCCTGCCACTGGACACAGGATAAGGCCCAGCGCACAGGCCC | |
| | CCACGTGGACACC | |
| Human | GCCCAGGCTGGAGTGCAGTGGCACAGTCACAACTCACTGCAGCCTC | 93 |
| Platelet | AAACTCCTGGGCTCAAAACGATCCACAGTCTCCTGAGTAGCTGGGA | |
| Derived | CTACAGGAGCTTGTTACCACACCCAGCTCCAGTTTATAAATTCATCT | |
| Growth | CCAGTTTATAAAGGAGGAAACCGAGGTACTGAGAGGTTAAAAAAC | |
| Factor | CTTCCTGCAGACACTTGTCCAGCAAGTGGCCACTCCAGGATTTGGA | |
| Subunit B | CCAAGGTGATGTGTCTTCAGGCTGTGTCTCTGCCACTGTGCCACGCT | |
| (hPDGFB | GCTGGGTGGTAGGCAGCAGTGGGTGGGTGCCTGCAGTGGTCTGTAA | |
| | AGACCACCTGAGATGTCCTTCCTCCTCTGTTCCACCCTGTCCAGGTC | |
| | CAAGAAGACAGTCTATGAAGAGAGAGCAGGTGTGACTCTCTCAGT | |
| | GTGCTCCTCTGTGAGAAGCAGGCTGACATCCCAAAGGGAAGGGCG | |
| | GATAACAGAGACAGTGCAAGCGGAGGAGATGAGGGTGCCTCAAAG | |
| | CCGGGAGGCTGGGTGATGCAGGAGCCTGCGTGTCCCGAGGGGGGT | |
| | GCTGGGCCCAGTGTGAGTACGTGTGACTGTGACTGAGACAGTGTGA | |
| | CTGCTGAAGGCAGGGACACAGCAGCTCCCTGACTGGGGGCAGAAG | |
| | GCGTTAACTGTGTGAAGGCTGGTTGTGGGTGGGTGGGCTCTGGGCC | |
| | TCGAACCCGGGGGCTGAGGGAGATAGTAAACAGCAGGGTGACTGA | |
| | CGGGAAGATCATGTTGGTAGCCCTGCGAAGATGCTGCAGGGCTGTG | |
| | GGGGTTTGTGTGACTTTGCAGTTCAACAAATTCAAATTCAGCCAAC | |
| | GCTGGCAGGGCCTGTTGTGCCAGGCAACCAGCTAGGAGGAGGAGA | |
| | CTCGGACCCAGCTTGCAGCTGAAGGGCGCTGGCTGCCGGGTTCTGT | |
| | GGGTTCACCTTGCGGTGTCTTCCCTTGCTAACACTGAGTCCTTACAA | |
| | TAGCCCCATCTCCAGGTTGAGGCTAGATGGAGGGGACAGAGGGAA | |
| | GTGACTTGCCCAAGGTGACCCAAGCTCCCGAGTGCCAGGGCAGGAT | |
| | CTGAATTCAGGCTCTCAGACTGCAGAGCCTGAGTCCCTCCCTGCCA | |
| | TGCCTGTGCCAGGGTGGAAATGTCTGGTCCTGGAGGGGAGCGTGGA | |
| | CTCCTGGCCTTGGCTCTGGAGACATCCCCCTAGACCACGTGGGCTC | |
| | CTAACCTGTCCATGGTCACTGTGCTGAGGGGGGGGACGGTGGGTCA | |
| | CCCCTAGTTCTTTTTTCCCCAGGGCCAGATTCATGGACTGAAGGGTT | |
| | GCTCGGCTCTCAGAGACCCCCTAAGCGCCCCGCCCTGGCCCCAAGC | |
| | CCTCCCCCAGCTCCCGCGTCCCCCCCCTCCTGGCGCTGACTCCGGGC | |
| | CAGAAGAGGAAAGGCTGTCTCCACCCACCTCTCGCACTCTCCCTTC | |
| | TCCTTTATAAAGGCCGGAACAGCTGAAAGGGTGGCAACTTCTCCTC | |
| | CTGCAGCCGGGAGCGGCCTGCCTGCCTCCCTGCGCACCCGCAGCCT | |
| | CCCCCGCTGCCTCCCTAGGGCTCCCCTCCGGCCGCCAGCGCCCATTT | |
| | TTCATTCCCTAGATAGAGATACTTTGCGCGCACACACATACATACG | |
| | CGCGCAAAAAGGAAAAAAAAAAAAAAAAGCCCACCCTCCAGCCTC | |
| | GCTGC | |
| Human | ACATCCAATGCCCGCTCTGCCTCATCTTCTATGGGAAACAAGAATT | 94 |
| Endothelial | TTAGAGGTCAGGTAGCCTAACACCATCAATTCTCAAAAGAGGAAGC | |
| Cell | TGAGGCCAAGAGAAGTCCTGTGAATTTCTTACAGCTCATTTGTGAC | |
| Specific | AGACCAAGAATTACCCACTTTACTGGGTTGTTATTTACTAAGTGAC | |
| Molecule | AGTGAGTCTATATCTCTTTTGACAAGTGAGGTGGGGGCATGGAATT | |
| 1 (hESM1) | CGGCATGTGGTTGGTGTAAGAACTCCCCTCTCTCCTCTTTAACCTTA | |
| | CTTAATAAGACCCTGGCACAGTTGATATTTTAAGAGGGCTACTCTG | |
| | TTTTCCCAGAGGGACCTAGGCACGGTAACCCTCTTAGCATGCAGAC | |
| | CTTGTTTCCTGAGGGGTAATGTTTCCCTTCCCTGTGACTTGTTTCTTG | |
| | GGGGCTGTGTTCTGATTTTCCTGCTGAGCCACTTGTTGCCTTGGGCT | |
| | GGCTGCCGCGCTTGGCAGTTTTTAGTGAGGGCTCTGATAGATGCCA | |
| | GGAGGTGAGGGGAAGGGCTCTGGGTGGACTCCGTCATTGGACAAG | |
| | CAGACTTAGTGATGGATGAGCCTTCCCCTGAGGAAGTTTTGGATCA | |
| | GAAGTCCAACTGATAAGTTTTTCCAGAATTGAGTAACCCAGAAGCA | |
| | GTGCCGAAAGGATCTTACCTCTCTTGTGGCTTTTTGTATTGATTTTA | |
| | AAAGAAATTCTCAGAGGCAGTTCCACATTGTACTGGAAGCACAGCT | |
| | ATATCCACAATAGGCTTAGATATATGTAACATGAATTGCTTTAGAA | |
| | ATAACATTTGAGGAGAGGGGTGAGAGGAAGGAAGAGAGGGTCTTA | |
| | AAAAATAGCCCTATCAAAATATTTTCTTTCTTCTAAGTATTGAAAAG | |
| | ACACAATATAACCCTTTCTTCTTTCAAATGATCTCATAGCTATTTGT | |
| | TGAGGGGAAATACCAAATGTTTATTATTTTTTTTGAAGAAGCTTCTT | |
| | CGGTCCTGATGATTCATGTTGATATCATTTTCCTCCTGACTACAGAG | |
| | GCTCTGAGACAAAGCTACACCTCAAGTGATATGCCAGGGTCAGAAC | |
| | AATTCCCGTCCTGAAGGAGGGTGTGCAACCTTCTTTATCCCTCCTTC | |
| | ACAGACGTCCTTGAGCCCTTGAGACGGATGTGAGTGAGTTTTTCAG | |
| | TCCTCATGCAAAACAACCATCTAAACATAACAGATGACATCAGCTT | |
| | GGGCTTTTCAATTCCTGGATGGCAGCAGCGTGTTAATCCAGCCTTC | |
| | ATCCTGGATTTCATAAACCAAAACAAGAGAGCCTGGCAGGAGGAC | |
| | AGCGCTGCTGCTGGGTTGAGGAAATTGATGACGGGAAAGCATGCG | |
| | GGCAACCCAGTGTATAAAACTCATAAACGTGTAGGCAGAGGCTCA | |
| | GCTACCAGTTTGGACGGCTGCTTCCCACCAGCAAAGACCACGACTG | |
| | GAGAGCCGAGCCGGAGGCAGCTGG | |
| Human Apelin | TGGCACACACGCACCCTGTCCAATGTATCTTTTGTGTAAATCTGGAC | 95 |
| (hAPLN) | TTAACACTTCAAGCAAACTGCCTGGCTTGCTGAAAGGTGGAGACAC | |
| | CTTTCGATTCAGTCTTTTAATATGTGTTGAGTGCCACCTATGTGCAG | |
| | AGCAAGATATTGGGGACTTTGGAGAGATCCAGAAGAGTGAGAAGA | |
| | CAGTATCCTACCTTAGGGGGTTCCCAGTCCAATGAGGGAAGCAGCC | |
| | CCATGCCTTGGGAGCTCCCAAGCTATAGAAGCAGCTAACAATCGAG | |
| | TCTGGAAAGGCAAACAACTTCAGGACCCGCTTCTAAAGCGGAATCG | |
| | CAAGTACACGCAAAATGAATCCAGCCTTGACTGTGTGGAGTTGGGT | |
| | AAACCACCTGCCTCTTACGTTGATGGGGAACTAGAATGAGGACAGC | |
| | TCCAGGGAACAAGAAAGGGTAGACCATAGGAGCTGTCCCATGTCC | |
| | CAACAGTGGGGAGGAGCTGATGGGCGGCCCCTGCTGGATTAGTGTT | |
| | ATCCTGAGAAGGCTTCTGGATGCGATGGGATTTGAGGTGCTGCTGC | |
| | AAAGAATGAATTGCTCACGGAAGGGTGGGGTGGGGGCATTCCAGG | |
| | TAGAGGGTGCCTCCTGGGGGATGCAGGGAACATGAGGGGCCTGGG | |
| | CAATTAATCAAGCCTTGGGCACAAGCCTAGGCAGTCACCCCCAATT | |
| | CAAAGCCAGTTGAAAATGCAGAGGAGAGAGGAGGGCCAGTGTTTG | |
| | GTTGTCTTGACCAAACCCTTGAAGCTGGCCAGCGGCAAGGGCAAGG | |
| | ACCAGGGTCAGAGGTAGAGGGCGTGAGTGAAGGCAACCCAGACTG | |
| | AGTCCTTCCCTAAGCGCCCAGGTTTCCTGACAGCTGTTAAGGAAGC | |
| | AAGGTGAGAAAGGGTTAAGTGTGCCCCTCCACCGCCCCAAATGCTT | |
| | CCTGTGTTTGAAATCCTTCAGGTCTCTGCAAACCCTCTGGCCCCCGG | |
| | CCAGGCGGGCATTGTCCGGGGAGCGGTTGTAGGTTGTCAGAGAGG | |
| | CCGCGCAGCCTTTGTTGTGGGGCCACCTCGGGGTTCCCTCTCGCGCT | |
| | CACGCTCGGGCTGGGGCTGCAGAGTGCGTGCCTGGAGGGGGGCGG | |
| | TGCGGGAGGCTCGCTCCCTCTCCCTCTTCCTGCCCCCCCTCTAGCCC | |
| | TCCCGATGACCACATGACCAAGTGGGCTCGCGGCCAAGCCACAAG | |
| | CTACAAAATGCAGCCCCTGGAGTGAGCGGGGAGCATTCTCTCTGGC | |
| | AGCCGGGGTCACGGGCAGTTGCAGCCGCGGCCGAGCAGCCAGCCG | |
| | CTAAGAAAGAGCTCGCCGCTGCCGCTCCCGGAGCCGCCGAGGCCA | |
| | GCTTCGCGGCGCTGCCCCGCGGCGGGAGAGGAGGCTGCAGAAGAG | |
| | CGGAGGCGGCCAGCGG | |
| Human | GCCCAGCACCCCAAGGCGGCCAACGCCAAAACTCTCCCTCCTCCTC | 39 |
| beta-actin | TTCCTCAATCTCGCTCTCGCTCTTTTTTTTTTTCGCAAAAGGAGGGG | |
| (HuBa) | AGAGGGGGTAAAAAAATGCTGCACTGTGCGGCGAAGCCGGTGAGT | |
| | GAGCGGCGCGGGGCCAATCAGCGTGCGCCGTTCCGAAAGTTGCCTT | |
| | TTATGGCTCGAGCGGCCGCGGCGGCGCCCTATAAAACCCAGCGGCG | |
| | CGACGCGCCACCACCGCCGAGTC | |
| Chicken beta- | GGTCGAGGTGAGCCCCACGTTCTGCTTCACTCTCCCCATCTCCCCCC | 40 |
| actin (CBA) | CCTCCCCACCCCCAATTTTGTATTTATTTATTTTTTAATTATTTTGTG | |
| | CAGCGATGGGGGCGGGGGGGGGGGGGGCGCGCGCCAGGCGGGGC | |
| | GGGGCGGGGCGAGGGGCGGGGGGGGCGAGGCGGAGAGGTGCGG | |
| | CGGCAGCCAATCAGAGCGGCGCGCTCCGAAAGTTTCCTTTTATGGC | |
| | GAGGCGGCGGCGGCGGCGGCCCTATAAAAAGCGAAGCGCGCGGCG | |
| | GGCGGGA | |
| Cytomega- | TGGTGATGCGGTTTTGGCAGTACACCAATGGGCGTGGATAGCGGTT | 41 |
| lovirus | TGACTCACGGGGATTTCCAAGTCTCCACCCCATTGACGTCAATGGG | |
| (CMV) | AGTTTGTTTTGGCACCAAAATCAACGGGACTTTCCAAAATGTCGTA | |
| | ATAACCCCGCCCCGTTGACGCAAATGGGCGGTAGGCGTGTACGGTG | |
| | GGAGGTCTATATAAGCAGAGCTCGTTTAGTGAACCG | |
| Cytomega- | TAGTTATTAATAGTAATCAATTACGGGGTCATTAGTTCATAGCCCAT | 42 |
| lovirus | ATATGGAGTTCCGCGTTACATAACTTACGGTAAATGGCCCGCCTGG | |
| (CMV) | CTGACCGCCCAACGACCCCCGCCCATTGACGTCAATAATGACGTAT | |
| (second | GTTCCCATAGTAACGCCAATAGGGACTTTCCATTGACGTCAATGGG | |
| version) | TGGAGTATTTACGGTAAACTGCCCACTTGGCAGTACATCAAGTGTA | |
| | TCATATGCCAAGTACGCCCCCTATTGACGTCAATGACGGTAAATGG | |
| | CCCGCCTGGCATTATGCCCAGTACATGACCTTATGGGACTTTCCTAC | |
| | TTGGCAGTACATCTACGTATTAGTCATCGCTATTACCATGGTGATGC | |
| | GGTTTTGGCAGTACATCAATGGGCGTGGATAGCGGTTTGACTCACG | |
| | GGGATTTCCAAGTCTCCACCCCATTGACGTCAATGGGAGTTTGTTTT | |
| | GGCACCAAAATCAACGGGACTTTCCAAAATGTCGTAACAACTCCGC | |
| | CCCATTGACGCAAATGGGCGGTAGGCGTGTACGGTGGGAGGTCTAT | |
| | ATAAGCAGAGCTGGTTTAGTGAACCGT | |
| Cytomega- | CGTTACATAACTTACGGTAAATGGCCCGCCTGGCTGACCGCCCAAC | 43 |
| lovirus | GACCCCCGCCCATTGACGTCAATAATGACGTATGTTCCCATAGTAA | |
| (CMV) | CGCCAATAGGGACTTTCCATTGACGTCAATGGGTGGAGTATTTACG | |
| (third | GTAAACTGCCCACTTGGCAGTACATCAAGTGTATCATATGCCAAGT | |
| version) | ACGCCCCCTATTGACGTCAATGACGGTAAATGGCCCGCCTGGCATT | |
| | ATGCCCAGTACATGACCTTATGGGACTTTCCTACTTGGCAGTACATC | |
| | TACGTATTAGTCATCGCTATTACCATGGTGATGCGGTTTTGGCAGTA | |
| | CATCAATGGGCGTGGATAGCGGTTTGACTCACGGGGATTTCCAAGT | |
| | CTCCACCCCATTGACGTCAATGGGAGTTTGTTTTGGCACCAAAATC | |
| | AACGGGACTTTCCAAAATGTCGTAACAACTCCGCCCCATTGACGCA | |
| | AATGGGCGGTAGGCGTGTACGGTGGGAGGTCTATATAAGCAGAGCT | |
| CAG promoter | ACTTACGGTAAATGGCCCGCCTGGCTGACCGCCCAACGACCCCCGC | 44 |
| (first | CCATTGACGTCAATAATGACGTATGTTCCCATAGTAACGCCAATAG | |
| version) | GGACTTTCCATTGACGTCAATGGGTGGAGTATTTACGGTAAACTGC | |
| | CCACTTGGCAGTACATCAAGTGTATCATATGCCAAGTACGCCCCCT | |
| | ATTGACGTCAATGACGGTAAATGGCCCGCCTGGCATTATGCCCAGT | |
| | ACATGACCTTATGGGACTTTCCTACTTGGCAGTACATCTACGTATTA | |
| | GTCATCGCTATTACCATGGTCGAGGTGAGCCCCACGTTCTGCTTCAC | |
| | TCTCCCCATCTCCCCCCCCTCCCCACCCCCAATTTTGTATTTATTTAT | |
| | TTTTTAATTATTTTGTGCAGCGATGGGGGCGGGGGGGGGGGGGGCG | |
| | CGCGCCAGGCGGGGCGGGGCGGGGCGAGGGGCGGGGCGGGGCGA | |
| | GGCGGAGAGGTGCGGCGGCAGCCAATCAGAGCGGCGCGCTCCGAA | |
| | AGTTTCCTTTTATGGCGAGGCGGCGGCGGCGGCGGCCCTATAAAAA | |
| | GCGAAGCGCGCGGCGGGCGG | |
| CAG promoter | CGTTACATAACTTACGGTAAATGGCCCGCCTGGCTGACCGCCCAAC | 45 |
| (second | GACCCCCGCCCATTGACGTCAATAATGACGTATGTTCCCATAGTAA | |
| version) | CGCCAATAGGGACTTTCCATTGACGTCAATGGGTGGAGTATTTACG | |
| | GTAAACTGCCCACTTGGCAGTACATCAAGTGTATCATATGCCAAGT | |
| | ACGCCCCCTATTGACGTCAATGACGGTAAATGGCCCGCCTGGCATT | |
| | ATGCCCAGTACATGACCTTATGGGACTTTCCTACTTGGCAGTACATC | |
| | TACGTATTAGTCATCGCTATTACCATGTCGAGGTGAGCCCCACGTTC | |
| | TGCTTCACTCTCCCCATCTCCCCCCCCTCCCCACCCCCAATTTTGTAT | |
| | TTATTTATTTTTTAATTATTTTGTGCAGCGATGGGGGCGGGGGGGGG | |
| | GGGGGCGCGCGCCAGGCGGGGCGGGGCGGGGCGAGGGGCGGGGC | |
| | GGGGCGAGGCGGAGAGGTGCGGCGGCAGCCAATCAGAGCGGCGCG | |
| | CTCCGAAAGTTTCCTTTTATGGCGAGGCGGCGGCGGCGGCGGCCCT | |
| | ATAAAAAGCGAAGCGCGCGGCGGGCG | |
| Human | CAACCTTTGGAGCTAAGCCAGCAATGGTAGAGGGAAGATTCTGCAC | 46 |
| EF1-alpha | GTCCCTTCCAGGCGGCCTCCCCGTCACCACCCCCCCCAACCCGCCC | |
| (EF1-α) | CGACCGGAGCTGAGAGTAATTCATACAAAAGGACTCGCCCCTGCCT | |
| | TGGGGAATCCCAGGGACCGTCGTTAAACTCCCACTAACGTAGAACC | |
| | CAGAGATCGCTGCGTTCCCGCCCCCTCACCCGCCCGCTCTCGTCATC | |
| | ACTGAGGTGGAGAATAGCATGCGTGAGGCTCCGGTGCCCGTCAGTG | |
| | GGCAGAGCGCACATCGCCCACAGTCCCCGAGAAGTTGGGGGGAGG | |
| | GGTCGGCAATTGAACGGGTGCCTAGAGAAGGTGGCGCGGGGTAAA | |
| | CTGGGAAAGTGATGTCGTGTACTGGCTCCGCCTTTTTCCCGAGGGT | |
| | GGGGGAGAACCGTATATAAGTGCAGTAGTCGCCGTGAACGTT | |
| Human | AGTGCAAGTGGGTTTTAGGACCAGGATGAGGCGGGGTGGGGGTGC | 47 |
| Synapsin1 | CTACCTGACGACCGACCCCGACCCACTGGACAAGCACCCAACCCCC | |
| (Syn), | ATTCCCCAAATTGCGCATCCCCTATCAGAGAGGGGGAGGGGAAAC | |
| short | AGGATGCGGCGAGGCGCGTGCGCACTGCCAGCTTCAGCACCGCGG | |
| version | ACAGTGCCTTCGCCCCCGCCTGGCGGCGCGCGCCACCGCCGCCTCA | |
| | GCACTGAAGGCGCGCTGACGTCACTCGCCGGTCCCCCGCAAACTCC | |
| | CCTTCCCGGCCACCTTGGTCGCGTCCGCGCCGCCGCCGGCCCAGCC | |
| | GGACCGCACCACGCGAGGCGCGAGATAGGGGGGCACGGGCGCGAC | |
| | CATCTGCGCTGCGGCGCCGGCGACTCAGCGCTGCCTC | |
| Human | AGTGCAAGTGGGTTTTAGGACCAGGATGAGGCGGGGTGGGGGTGC | 48 |
| Synapsin1 | CTACCTGACGACCGACCCCGACCCACTGGACAAGCACCCAACCCCC | |
| (Syn) | ATTCCCCAAATTGCGCATCCCCTATCAGAGAGGGGGAGGGGAAAC | |
| with 3′ | AGGATGCGGCGAGGCGCGTGCGCACTGCCAGCTTCAGCACCGCGG | |
| extension | ACAGTGCCTTCGCCCCCGCCTGGCGGCGCGCGCCACCGCCGCCTCA | |
| | GCACTGAAGGCGCGCTGACGTCACTCGCCGGTCCCCCGCAAACTCC | |
| | CCTTCCCGGCCACCTTGGTCGCGTCCGCGCCGCCGCCGGCCCAGCC | |
| | GGACCGCACCACGCGAGGCGCGAGATAGGGGGGCACGGGCGCGAC | |
| | CATCTGCGCTGCGGCGCCGGCGACTCAGCGCTGCCTCAGTCTGCGG | |
| | TGGGCAGCGGAGGAGTCGTGTCGTGCCTGAGAGCGCAG | |
| Human Synapsin1 | CTGCAGAGGGCCCTGCGTATGAGTGCAAGTGGGTTTTAGGACCAGG | 49 |
| (Syn) with 5′ | ATGAGGCGGGGTGGGGGTGCCTACCTGACGACCGACCCCGACCCA | |
| extension | CTGGACAAGCACCCAACCCCCATTCCCCAAATTGCGCATCCCCTAT | |
| | CAGAGAGGGGGAGGGGAAACAGGATGCGGCGAGGCGCGTGCGCA | |
| | CTGCCAGCTTCAGCACCGCGGACAGTGCCTTCGCCCCCGCCTGGCG | |
| | GCGCGCGCCACCGCCGCCTCAGCACTGAAGGCGCGCTGACGTCACT | |
| | CGCCGGTCCCCCGCAAACTCCCCTTCCCGGCCACCTTGGTCGCGTCC | |
| | GCGCCGCCGCCGGCCCAGCCGGACCGCACCACGCGAGGCGCGAGA | |
| | TAGGGGGGCACGGGCGCGACCATCTGCGCTGCGGCGCCGGCGACT | |
| | CAGCGCTGCCTC | |
| Human CamKIIa | ACTTGTGGACAAAGTTTGCTCTATTCCACCTCCTCCAGGCCCTCCTT | 50 |
| (CaMKIIa) | GGGTCCATCACCCCAGGGGTGCTGGGTCCATCCCACCCCCAGGCCC | |
| | ACACAGGCTTGCAGTATTGTGTGCGGTATGGTCAGGGCGTCCGAGA | |
| | GCAGGTTTCGCAGTGGAAGGCAGGCAGGTGTTGGGGAGGCAGTTA | |
| | CCGGGGCAACGGGAACAGGGCGTTTTGGAGGTGGTTGCCATGGGG | |
| | ACCTGGATGCTGACGAAGGCTCGCGAGGCTGTGAGCAGCCACAGT | |
| | GCCCTGC | |
| eSYN | GACATTGATTATTGACTAGTTATTAATAGTAATCAATTACGGGGTC | 51 |
| promoter | ATTAGTTCATAGCCCATATATGGAGTTCCGCGTTACATAACTTACG | |
| | GTAAATGGCCCGCCTGGCTGACCGCCCAACGACCCCCGCCCATTGA | |
| | CGTCAATAATGACGTATGTTCCCATAGTAACGCCAATAGGGACTTT | |
| | CCATTGACGTCAATGGGTGGACTATTTACGGTAAACTGCCCACTTG | |
| | GCAGTACATCAAGTGTATCATATGCCAAGTACGCCCCCTATTGACG | |
| | TCAATGACGGTAAATGGCCCGCCTGGCATTATGCCCAGTACATGAC | |
| | CTTATGGGACTTTCCTACTTGGCAGTACATCTACGTATTAGTCATCG | |
| | CTATTACCATGGCTGCAGAGGGCCCTGCGTATGAGTGCAAGTGGGT | |
| | TTTAGGACCAGGATGAGGCGGGGTGGGGGTGCCTACCTGACGACC | |
| | GACCCCGACCCACTGGACAAGCACCCAACCCCCATTCCCCAAATTG | |
| | CGCATCCCCTATCAGAGAGGGGGAGGGGAAACAGGATGCGGCGAG | |
| | GCGCGTCGCGACTGCCAGCTTCAGCACCGCGGACAGTGCCTTCGCC | |
| | CCCGCCTGGCGGCGCGCGCCACCGCCGCCTCAGCACTGAAGGCGCG | |
| | CTGACGTCACTCGCCGGTCCCCCGCAAACTCCCCTTCCCGGCCACCT | |
| | TGGTCGCGTCCGCGCCGCCGCCGGCCCAGCCGGACCGCACCACGCG | |
| | AGGCGCGAGATAGGGGGGCACGGGCGCGACCATCTGCGCTGCGGC | |
| | GCCGGCGACTCAGCGCTGCCTCAGTCTGCGGTGGGCAGCGGAGGA | |
| | GTCGTGTCGTGCCTGAGAGCGCAGG | |
| hGLUT1 | ACCATTTTGCTAGAGAAGGCCGCGGAGGCTCAGAGAGGTGCGCAC | 102 |
| promoter | ACTTGCCCTGAGTCACACAGCGAATGCCCTCCGCGGTCCCAACGCA | |
| | GAGAGAACGAGCCGATCGGCAGCCTGAGCGAGGCAGTGGTTAGGG | |
| | GGGGCCCCGGCCCCGGCCACTCCCCTCACCCCCTCCCCGCAGAGCG | |
| | CCGCCCAGGACAGGCTGGGCCCCAGGCCCCGCCCCGAGGTCCTGCC | |
| | CACACACCCCTGACACACCGGCGTCGCCAGCCAATGGCCGGGGTCC | |
| | TATAAACGCTACGGTCCGCGCGCTCTCT | |

In some embodiments, the AAV virion genome comprises a polynucleotide sequence at least 75%, at least 80%, at least 85%, at least 900%, at least 92%, at least 93%, at least 94% at least 95% at least 96%, at least 97% at least 98%, at least 99% or 100% identical to SEQ TD NO: 1. In some embodiments, the AAV virion genome comprises a polynucleotide sequence at least 75% at least 80%, at least 85%, at least 90%, at least 92%, at least 93% at least 94%, at least 95%, at least 96%, at least 97%, at least 98%, at least 99% or 100% identical to SEQ TD NO: 2. In some embodiments, the AAV virion genome comprises a polynucleotide sequence at least 75%, at least 80%, at least 85%, at least 90%, at least 92%, at least 93% at least 94%, at least 95%, at least 96%, at least 97%, at least 98%, at least 99%, or 100% identical to SEQ ID NO: 3.

Further illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. A large variety of other promoters are known and generally available in the art, and the sequences of many such promoters are available in sequence databases such as the GenBank database.

### Other Regulatory Elements

In some cases, AAV virions of the present disclosure further comprise one or more regulatory elements selected from the group consisting of an enhancer, an intron, a poly-A signal, a 2A peptide encoding sequence, a WPRE (Woodchuck hepatitis virus posttranscriptional regulatory element), and a HPRE (Hepatitis B posttranscriptional regulatory element).

In some embodiments, the rAAV virion genome comprises a CMV enhancer.

In certain embodiments, the rAAV virion genome comprises one or more enhancers. In particular embodiments, the enhancer is a CMV enhancer sequence, a GAPDH enhancer sequence, a 3-actin enhancer sequence, or an EF1-α enhancer sequence. Sequences of the foregoing are known in the art. For example, the sequence of the CMV immediate early (IE) enhancer is:

(SEQ ID NO: 52)
CGTTACATAACTTACGGTAAATGGCCCGCCTGGCTGACCGCCCAACGAC
CCCCGCCCATTGACGTCAATAATGACGTATGTTCCCATAGTAACGCCAA
TAGGGACTTTCCATTGACGTCAATGGGTGGAGTATTTACGGTAAACTGC
CCACTTGGCAGTACATCAAGTGTATCATATGCCAAGTACGCCCCCTATT
GACGTCAATGACGGTAAATGGCCCGCCTGGCATTATGCCCAGTACATGA
CCTTATGGGACTTTCCTACTTGGCAGTACATCTACGTATTAGTCATCGC
TATTACCATG

In certain embodiments, the rAAV virion genome comprises one or more introns. In particular embodiments, the intron is a rabbit globin intron sequence, a chicken 3-actin intron sequence, a synthetic intron sequence, or an EF1-α intron sequence.

In certain embodiments, the rAAV virion genome comprises a polyA sequence. In particular embodiments, the polyA sequence is a rabbit globin polyA sequence, a human growth hormone polyA sequence, a bovine growth hormone polyA sequence, a PGK polyA sequence, an SV40 polyA sequence, or a TK polyA sequence. In some embodiments, the poly-A signal may be a bovine growth hormone polyadenylation signal (bGHpA).

In certain embodiments, the rAAV virion genome comprises one or more transcript stabilizing element. In particular embodiments, the transcript stabilizing element is a WPRE sequence, a HPRE sequence, a scaffold-attachment region, a 3′ UTR, or a 5′ UTR. In particular embodiments, the rAAV virion genome comprises both a 5′ UTR and a 3′ UTR.

In some embodiments, the rAAV virion genome comprises a 5′ untranslated region (UTR) selected from Table 4. In some embodiments, the rAAV virion genome comprises a polynucleotide sequence at least 75%, 80%, 85%, 90%, 92%, 93%, 94%, 95% 96%, 97%, 98%, 99%, or 100% identical to any one of SEQ TD NOS 53-61.

TABLE 4

| 5′ | | SEQ |
|---|---|---|
| UNTRANSLATED | | ID |
| REGION | SEQUENCE | NO: |
| Human beta-actin | CGCGTCCGCCCGCGAGCACAGAGCCTCGCCTTTGCCGATC | 53 |
| exon/intron | CGCCGCCCGTCCACACCCGCCGCCAGGTAAGCCCGGCCAG | |
| | CCGACCGGGGCATGCGGCCGCGGCCCTTCGCCCGTGCAGA | |
| | GCCGCCGTCTGGGCCGCAGCGGGGGGCGCATGGGGCGGA | |
| | ACCGGACCGCCGTGGGGGGCGCGGGAGAAGCCCCTGGGC | |
| | CTCCGGAGATGGGGGACACCCCACGCCAGTTCGCAGGCG | |
| | CGAGGCCGCGCTCGGGCGGGCGCGCTCCGGGGGTGCCGC | |
| | TCTCGGGGCGGGGGCAACCGGCGGGGTCTTTGTCTGAGCC | |
| | GGGCTCTTGCCAATGGGGATCGCACGGTGGGCGCGGCGTA | |
| | GCCCCCGTCAGGCCCGGTGGGGGCTGGGGCGCCATGCGC | |
| | GTGCGCGCTGGTCCTTTGGGCGCTAACTGCGTGCGCGCTG | |
| | GGAATTGGCGCTAATTGCGCGTGCGCGCTGGGACTCAATG | |
| | GCGCTAATCGCGCGTGCGTTCTGGGGCCCGGGCGCTTGCG | |
| | CCACTTCCTGCCCGAGCCGCTGGCGCCCGAGGGTGTGGCC | |
| | GCTGCGTGCGCGCGCGCGACCCGGTCGCTGTTTGAACCGG | |
| | GCGGAGGCGGGGCTGGCGCCCGGTTGGGAGGGGGTTGGG | |
| | GCCTGGCTTCCTGCCGCGCGCCGCGGGGACGCCTCCGACC | |
| | AGTGTTTGCCTTTTATGGTAATAACGCGGCCGGCCCGGCT | |
| | TCCTTTGTCCCCAATCTGGGCGCGCGCCGGCGCCCCCTGG | |
| | CGGCCTAAGGACTCGGCGCGCCGGAAGTGGCCAGGGCGG | |
| | CAGCGGCTGCTCTTGGCGGCCCCGAGGTGACTATAGCCTT | |
| | CTTTTGTGTCTTGATAGTTCGCCAGCCTCTGCTAACCATGT | |
| | TCATGCCTTCTTCTTTTTCCTACAGCTCCTGGGCAACGTGC | |
| | TGGTTATTGTGCTGTCTCATCATTTTGGCAAAGAATTC | |
| Chicken beta-actin | GTCGCTGCGCGCTGCCTTCGCCCCGTGCCCCGCTCCGCCG | 54 |
| exon/intron + rabbit | CCGCCTCGCGCCGCCCGCCCCGGCTCTGACTGACCGCGTT | |
| globin intron | ACTCCCACAGGTGAGCGGGCGGGACGGCCCTTCTCCTCCG | |
| | GGCTGTAATTAGCGCTTGGTTTAATGACGGCTTGTTTCTTT | |
| | TCTGTGGCTGCGTGAAAGCCTTGAGGGGCTCCGGGAGGGC | |
| | CCTTTGTGCGGGGGGAGCGGCTCGGGGGGTGCGTGCGTGT | |
| | GTGTGTGCGTGGGGAGCGCCGCGTGCGGCTCCGCGCTGCC | |
| | CGGCGGCTGTGAGCGCTGCGGGCGCGGCGCGGGGCTTTGT | |
| | GCGCTCCGCAGTGTGCGCGAGGGGAGCGCGGCCGGGGGC | |
| | GGTGCCCCGCGGTGCGGGGGGGGCTGCGAGGGGAACAAA | |
| | GGCTGCGTGCGGGGTGTGTGCGTGGGGGGGTGAGCAGGG | |
| | GGTGTGGGCGCGTCGGTCGGGCTGCAACCCCCCCTGCACC | |
| | CCCCTCCCCGAGTTGCTGAGCACGGCCCGGCTTCGGGTGC | |
| | GGGGCTCCGTACGGGGCGTGGCGCGGGGCTCGCCGTGCC | |
| | GGGCGGGGGGTGGCGGCAGGTGGGGGTGCCGGGCGGGGC | |
| | GGGGCCGCCTCGGGCCGGGGAGGGCTCGGGGGAGGGGCG | |
| | CGGCGGCCCCCGGAGCGCCGGCGGCTGTCGAGGCGCGGC | |
| | GAGCCGCAGCCATTGCCTTTTATGGTAATCGTGCGAGAGG | |
| | GCGCAGGGACTTCCTTTGTCCCAAATCTGTGCGGAGCCGA | |
| | AATCTGGGAGGCGCCGCCGCACCCCCTCTAGCGGGCGCGG | |
| | GGCGAAGCGGTGCGGCGCCGGCAGGAAGGAAATGGGCGG | |
| | GGAGGGCCTTCGTGCGTCGCCGCGCCGCCGTCCCCTTCTC | |
| | CCTCTCCAGCCTCGGGGCTGTCCGCGGGGGGACGGCTGCC | |
| | TTCGGGGGGGACGGGGCAGGGCGGGGTTCGGCTTCTGGC | |
| | GTGTGACCGGCGGCTCTAGAGCCTCTGCTAACCATGTTCA | |
| | TGCCTTCTTCTTTTTCCTACAGCTCCTGGGCAACGTGCTGG | |
| | TTATTGTGCTGTCTCATCATTTTGGCAAAGAATTC | |
| 5′ UTR-Syn1 Hs | AGTCTGCGGTGGGCAGCGGAGGAGTCGTGTCGTGCCTGAG | 55 |
| | AGCGCAGCTGTGCTCCTGGGCACCGCGCAGTCCGCCCCCG | |
| | CGGCTCCTGGCCAGACCACCCCTAGGACCCCCTGCCCCAA | |
| | GTCGCA | |
| CMV IE exon | TCAGATCGCCTGGAGAGGCCATCCACGCTGTTTTGACCTC | 56 |
| | CATAGTGGACACCGGGACCGATCCAGCCTCCGCGGCCGG | |
| | GAACGGTGCATTGGAACGCGGATTCCCCGTGCCAAGAGTG | |
| | AC | |
| TPL-eMLP | CTCACTCTCTTCCGCATCGCTGTCTGCGAGGGCCAGCTGTT | 57 |
| (adenovirus derived | GGGCTCGCGGTTGAGGACAAACTCTTCGCGGTCTTTCCAG | |
| enhancer element) | TACTCTTGGATCGGAAACCCGTCGGCCTCCGAACGGTACT | |
| | CCGCCACCGAGGGACCTGAGCGAGTCCGCATCGACCGGA | |
| | TCGGAAAACCTCTCGAGAAAGGCGTCTAACCAGTCACAGT | |
| | CGCAAGGTAGGCTGAGCACCGTGGCGGGCGGCAGCGGGT | |
| | GGCGGTCGGGGTTGTTTCTGGCGGAGGTGCTGCTGATGAT | |
| | GTAATTAAAGTAGGCGGTCTTGAGACGGCGGATGGTCGA | |
| | GGTGAGGTGTGGCAGGCTTGAGATCCAGCTGTTGGGGTGA | |
| | GTACTCCCTCTCAAAAGCGGGCATTACTTCTGCGCTAAGA | |
| | TTGTCAGTTTCCAAAAACGAGGAGGATTTGATATTCACCT | |
| | GGCCCGATCTGGCCATACACTTGAGTGACAATGACATCCA | |
| | CTTTGCCTTTCTCTCCACAGGTGTCCACTCCCAG | |
| Human EF1-α | CTTTTTCGCAACGGGTTTGCCGCCAGAACACAGGTAAGTG | 58 |
| intron/exon | CCGTGTGTGGTTCCCGCGGGCCTGGCCTCTTTACGGGTTAT | |
| | GGCCCTTGCGTGCCTTGAATTACTTCCACCTGGCTCCAGTA | |
| | CGTGATTCTTGATCCCGAGCTGGAGCCAGGGGCGGGCCTT | |
| | GCGCTTTAGGAGCCCCTTCGCCTCGTGCTTGAGTTGAGGC | |
| | CTGGCCTGGGCGCTGGGGCCGCCGCGTGCGAATCTGGTGG | |
| | CACCTTCGCGCCTGTCTCGCTGCTTTCGATAAGTCTCTAGC | |
| | CATTTAAAATTTTTGATGACGTGCTGCGACGCTTTTTTTCT | |
| | GGCAAGATAGTCTTGTAAATGCGGGCCAGGATCTGCACAC | |
| | TGGTATTTCGGTTTTTGGGCCCGCGGCCGGCGACGGGGCC | |
| | CGTGCGTCCCAGCGCACATGTTCGGCGAGGCGGGGCCTGC | |
| | GAGCGCGGCCACCGAGAATCGGACGGGGGTAGTCTCAAG | |
| | CTGGCCGGCCTGCTCTGGTGCCTGGCCTCGCGCCGCCGTG | |
| | TATCGCCCCGCCCTGGGCGGCAAGGCTGGCCCGGTCGGCA | |
| | CCAGTTGCGTGAGCGGAAAGATGGCCGCTTCCCGGCCCTG | |
| | CTCCAGGGGGCTCAAAATGGAGGACGCGGCGCTCGGGAG | |
| | AGCGGGCGGGTGAGTCACCCACACAAAGGAAAAGGGCCT | |
| | TTCCGTCCTCAGCCGTCGCTTCATGTGACTCCACGGAGTAC | |
| | CGGGCGCCGTCCAGGCACCTCGATTAGTTCTGGAGCTTTT | |
| | GGAGTACGTCGTCTTTAGGTTGGGGGGAGGGGTTTTATGC | |
| | GATGGAGTTTCCCCACACTGAGTGGGTGGAGACTGAAGTT | |
| | AGGCCAGCTTGGCACTTGATGTAATTCTCCTTGGAATTTG | |
| | GCCTTTTTGAGTTTGGATCTTGGTTCATTCTCAAGCCTCAG | |
| | ACAGTGGTTCAAAGTTTTTTTCTTCCATTTCAG | |
| Human EF1-α, intron | GTAAGTGCCGTGTGTGGTTCCCGCGGGCCTGGCCTCTTTA | 59 |
| A | CGGGTTATGGCCCTTGCGTGCCTTGAATTACTTCCACCTGG | |
| | CTGCAGTACGTGATTCTTGATCCCGAGCTTCGGGTTGGAA | |
| | GTGGGTGGGAGAGTTCGAGGCCTTGCGCTTAAGGAGCCCC | |
| | TTCGCCTCGTGCTTGAGTTGAGGCCTGGCCTGGGCGCTGG | |
| | GGCCGCCGCGTGCGAATCTGGTGGCACCTTCGCGCCTGTC | |
| | TCGCTGCTTTCGATAAGTCTCTAGCCATTTAAAATTTTTGA | |
| | TGACCTGCTGCGACGCTTTTTTTCTGGCAAGATAGTCTTGT | |
| | AAATGCGGGCCAAGATCTGCACACTGGTATTTCGGTTTTT | |
| | GGGGCCGCGGGCGGCGACGGGGCCCGTGCGTCCCAGCGC | |
| | ACATGTTCGGCGAGGCGGGGCCTGCGAGCGCGGCCACCG | |
| | AGAATCGGACGGGGGTAGTCTCAAGCTGGCCGGCCTGCTC | |
| | TGGTGCCTGGCCTCGCGCCGCCGTGTATCGCCCCGCCCTG | |
| | GGCGGCAAGGCTGGCCCGGTCGGCACCAGTTGCGTGAGC | |
| | GGAAAGATGGCCGCTTCCCGGCCCTGCTGCAGGGAGCTCA | |
| | AAATGGAGGACGCGGCGCTCGGGAGAGCGGGCGGGTGAG | |
| | TCACCCACACAAAGGAAAAGGGCCTTTCCGTCCTCAGCCG | |
| | TCGCTTCATGTGACTCCACGGAGTACCGGGCGCCGTCCAG | |
| | GCACCTCGATTAGTTCTCGAGCTTTTGGAGTACGTCGTCTT | |
| | TAGGTTGGGGGGAGGGGTTTTATGCGATGGAGTTTCCCCA | |
| | CACTGAGTGGGTGGAGACTGAAGTTAGGCCAGCTTGGCAC | |
| | TTGATGTAATTCTCCTTGGAATTTGCCCTTTTTGAGTTTGG | |
| | ATCTTGGTTCATTCTCAAGCCTCAGACAGTGGTTCAAAGTT | |
| | TTTTTCTTCCATTTCAG | |
| 5′ UTR human | TCAGAAGCCCCGGGCTCGTCAGTCAAACCGGTTCTCTGTT | 60 |
| CamKIIa | TGCACTCGGCAGCACGGGCAGGCAAGTGGTCCCTAGGTTC | |
| | GGG | |
| B-globin intron | GTGAGTCTATGGGACCCTTGATGTTTTCTTTCCCCTTCTTTT | 61 |
| | CTATGGTTAAGTTCATGTCATAGGAAGGGGAGAAGTAACA | |
| | GGGTACACATATTGACCAAATCAGGGTAATTTTGCATTTG | |
| | TAATTTTAAAAAATGCTTTCTTCTTTTAATATACTTTTTTGT | |
| | TTATCTTATTTCTAATACTTTCCCTAATCTCTTTCTTTCAGG | |
| | GCAATAATGATACAATGTATCATGCCTCTTTGCACCATTCT | |
| | AAAGAATAACAGTGATAATTTCTGGGTTAAGGCAATAGCA | |
| | ATATTTCTGCATATAAATATTTCTGCATATAAATTGTAACT | |
| | GATGTAAGAGGTTTCATATTGCTAATAGCAGCTACAATCC | |
| | AGCTACCATTCTGCTTTTATTTTATGGTTGGGATAAGGCTG | |
| | GATTATTCTGAGTCCAAGCTAGGCCCTTTTGCTAATCATGT | |
| | TCATACCTCTTATCTTCCTCCCACAG | |

In some embodiments, the rAAV virion genome comprises a 3′ untranslated region selected from Table 5. In some embodiments, the rAAV virion genome comprises a polynucleotide sequence at least 75%, 80%, 85%, 90%, 92%, 93%, 94%, 95% 96%, 97% 98%, 99%, or 100% identical to any one of SEQ TD NOS 62-70.

TABLE 5

| 3′ | | SEQ |
|---|---|---|
| UNTRANSLATED | | ID |
| REGION | SEQUENCE | NO: |
| WPRE(x) (mutated | AATCAACCTCTGGATTACAAAATTTGTGAAAGATTGACT | 62 |
| woodchuck hepatitis | GGTATTCTTAACTATGTTGCTCCTTTTACGCTATGTGGAT | |
| regulatory element- | ACGCTGCTTTAATGCCTTTGTATCATGCTATTGCTTCCCGT | |
| version 1) | ATGGCTTTCATTTTCTCCTCCTTGTATAAATCCTGGTTGCT | |
| | GTCTCTTTATGAGGAGTTGTGGCCCGTTGTCAGGCAACGT | |
| | GGCGTGGTGTGCACTGTGTTTGCTGACGCAACCCCCACTG | |
| | GTTGGGGCATTGCCACCACCTGTCAGCTCCTTTCCGGGAC | |
| | TTTCGCTTTCCCCCTCCCTATTGCCACGGCGGAACTCATC | |
| | GCCGCCTGCCTTGCCCGCTGCTGGACAGGGGCTCGGCTGT | |
| | TGGGCACTGACAATTCCGTGGTGTTGTCGGGGAAATCAT | |
| | CGTCCTTTCCTTGGCTGCTCGCCTGTGTTGCCACCTGGATT | |
| | CTGCGCGGGACGTCCTTCTGCTACGTCCCTTCGGCCCTCA | |
| | ATCCAGCGGACCTTCCTTCCCGCGGCCTGCTGCCGGCTCT | |
| | GCGGCCTCTTCCGCGTCTTCGCCTTCGCCCTCAGACGAGT | |
| | CGGATCTCCCTTTGGGCCGCCTCCCCGC | |
| WPRE(x) (mutated | TCAACCTCTGGATTACAAAATTTGTGAAAGATTGACTGGT | 63 |
| woodchuck hepatitis | ATTCTTAACTATGTTGCTCCTTTTACGCTATGTGGATACG | |
| regulatory element- | CTGCTTTAATGCCTTTGTATCATGCTATTGCTTCCCGTATG | |
| version 2) | GCTTTCATTTTCTCCTCCTTGTATAAATCCTGGTTGCTGTC | |
| | TCTTTATGAGGAGTTGTGGCCCGTTGTCAGGCAACGTGGC | |
| | GTGGTGTGCACTGTGTTTGCTGACGCAACCCCCACTGGTT | |
| | GGGGCATTGCCACCACCTGTCAGCTCCTTTCCGGGACTTT | |
| | CGCTTTCCCCCTCCCTATTGCCACGGCGGAACTCATCGCC | |
| | GCCTGCCTTGCCCGCTGCTGGACAGGGGCTCGGCTGTTGG | |
| | GCACTGACAATTCCGTGGTGTTGTCGGGGAAATCATCGTC | |
| | CTTTCCTTGGCTGCTCGCCTGTGTTGCCACCTGGATTCTGC | |
| | GCGGGACGTCCTTCTGCTACGTCCCTTCGGCCCTCAATCC | |
| | AGCGGACCTTCCTTCCCGCGGCCTGCTGCCGGCTCTGCGG | |
| | CCTCTTCCGCGTCTTCGCCTTCGCCCTCAGACGAGTCGGA | |
| | TCTCCCTTTGGGCCGCCTCCCCGCA | |
| WPRE(x) (mutated | TTCCTGTTAATCAACCTCTGGATTACAAAATTTGTGAAAG | 64 |
| woodchuck hepatitis | ATTGACTGGTATTCTTAACTATGTTGCTCCTTTTACGCTAT | |
| regulatory element- | GTGGATACGCTGCTTTAATGCCTTTGTATCATGCTATTGC | |
| version 3) | TTCCCGTATGGCTTTCATTTTCTCCTCCTTGTATAAATCCT | |
| | GGTTGCTGTCTCTTTATGAGGAGTTGTGGCCCGTTGTCAG | |
| | GCAACGTGGCGTGGTGTGCACTGTGTTTGCTGACGCAAC | |
| | CCCCACTGGTTGGGGCATTGCCACCACCTGTCAGCTCCTT | |
| | TCCGGGACTTTCGCTTTCCCCCTCCCTATTGCCACGGCGG | |
| | AACTCATCGCCGCCTGCCTTGCCCGCTGCTGGACAGGGG | |
| | CTCGGCTGTTGGGCACTGACAATTCCGTGGTGTTGTCGGG | |
| | GAAGCTGACGTCCTTTCCGCGGCTGCTCGCCTGTGTTGCC | |
| | ACCTGGATTCTGCGCGGGACGTCCTTCTGCTACGTCCCTT | |
| | CGGCCCTCAATCCAGCGGACCTTCCTTCCCGCGGCCTGCT | |
| | GCCGGCTCTGCGGCCTCTTCCGCCTCTTCGCCTTCGCCCT | |
| | CAGACGAGTCGGATCTCCCTTTGGGCCGCCTCCCCGCCCA | |
| | TGTATCTTTTTCACCTGTGCCTTGTTTTTGCCTGTGTTCCG | |
| | CGTCCTACTTTTCAAGCCTCCAAGCTGTGCCTTGGGCGGC | |
| | TTTGGGGCATGGACATAGATCCCTATAAAGAATTTGGTTC | |
| | ATCTTATCAGTTGTTGAATTTTCTTCCTTTGGAC | |
| CAAX | TGTGTGATAATG | 65 |
| EES | CTGTTCTCATCACATCATATCAAGGTTATATACCATCAAT | 66 |
| | ATTGCCACAGATGTTACTTAGCCTTTTAATATTTCTCTAAT | |
| | TTAGTGTATATGCAATGATAGTTCTCTGATTTCTGAGATT | |
| | GAGTTTCTCATGTGTAATGATTATTTAGAGTTTCTCTTTCA | |
| | TCTGTTCAAATTTTTGTCTAGTTTTATTTTTTACTGATTTG | |
| | TAAGACTTCTTTTTATAATCTGCATATTACAATTCTCTTTA | |
| | CTGGGGTGTTGCAAATATTTTCTGTCATTCTATGGCCTGA | |
| | CTTTTCTTAATGGTTTTTTAATTTTAAAAATAAGTCTTAAT | |
| | ATTCATGCAATCTAATTAACAATCTTTTCTTTGTGGTTAG | |
| | GACTTTGAGTCATAAGAAATTTTTCTCTACACTGAAGTCA | |
| | TGATGGCATGCTTCTATATTATTTTCTAAAAGATTTAAAG | |
| | TTTTGCCTTCTCCATTTAGACTTATAATTCACTGGAATTTT | |
| | TTTGTGTGTATGGTATGACATATGGGTTCCCTTTTATTTTT | |
| | TACATATAAATATATTTCCCTGTTTTTCTAAAAAAGAAAA | |
| | AGATCATCATTTTCCCATTGTAAAATGCCATATTTTTTTCA | |
| | TAGGTCACTTACATATATCAATGGGTCTGTTTCTGAGCTC | |
| | TACTCTATTTTATCAGCCTCACTGTCTATCCCCACACATCT | |
| | CATGCTTTGCTCTAAATCTTGATATTTAGTGGAACATTCT | |
| | TTCCCATTTTGTTCTACAAGAATATTTTTGTTATTGTCTTT | |
| | GGGCTTTCTATATACATTTTGAAATGAGGTTGACAAGTTA | |
| HPRE | ATAACAGGCCTATTGATTGGAAAGTTTGTCAACGAATTGT | 67 |
| | GGGTCTTTTGGGGTTTGCTGCCCCTTTTACGCAATGTGGA | |
| | TATCCTGCTTTAATGCCTTTATATGCATGTATACAAGCAA | |
| | AACAGGCTTTTACTTTCTCGCCAACTTACAAGGCCTTTCT | |
| | CAGTAAACAGTATATGACCCTTTACCCCGTTGCTCGGCAA | |
| | CGGCCTGGTCTGTGCCAAGTGTTTGCTGACGCAACCCCCA | |
| | CTGGTTGGGGCTTGGCCATAGGCCATCAGCGCATGCGTG | |
| | GAACCTTTGTGTCTCCTCTGCCGATCCATACTGCGGAACT | |
| | CCTAGCCGCTTGTTTTGCTCGCAGCAGGTCTGGAGCAAAC | |
| | CTCATCGGGACCGACAATTCTGTCGTACTCTCCCGCAAGT | |
| | ATACATCGTTTCCATGGCTGCTAGGCTGTGCTGCCAACTG | |
| | GATCCTGCGCGGGACGTCCTTTGTTTACGTCCCGTCGGCG | |
| | CTGAATCCCGCGGACGACCCCTCCCGGGGCCGCTTGGGG | |
| | CTCTACCGCCCGCTTCTCCGTCTGCCGTACCGTCCGACCA | |
| | CGGGGCGCACCTCTCTTTACGCGGACTCCCCGTCTGTGCC | |
| | TTCTCATCTGCCGGACCGTGTGCACTTCGCTTCACCTCTG | |
| | CACGTCGCATGGAGGCCACCGTGAACGCCCACCGGAACC | |
| | TGCCCAAGGTCTTGCATAAGAGGACTCTTGGACTTTCAGC | |
| | AATGTCATC | |
| R2V17 (HepB derived | TTCCTGTAAACAGGCCTATTGATTGGAAAGTTTGTCAACG | 68 |
| enhancer element) | AATTGTGGGTCTTTTGGGGTTTGCTGCCCCTTTTACGCAA | |
| | TGTGGATATCCTGCTTTAATGCCTTTATATGCATGTATAC | |
| | AAGCAAAACAGGCTTTTACTTTCTCGCCAACTTACAAGGC | |
| | CTTTCTCAGTAAACAGTATATGACCCTTTACCCCGTTGCT | |
| | CGGCAACGGCCTGGTCTGTGCCAAGTGTTTGCTGACGCA | |
| | ACCCCCACTGGTTGGGGCTTGGCCATAGGCCATCAGCGC | |
| | ATGCGTGGAACCTTTGTGTCTCCTCTGCCGATCCATACTG | |
| | CGGAACTCCTAGCCGCTTGTTTTGCTCGCAGCTGGACTGG | |
| | AGCAAACCTCATCGGGACCGACAATTCTGTCGTACTCTCC | |
| | CGCAAGCACTCACCGTTTCCGCGGCTGCTCGCCTGTGTTG | |
| | CCACCTGGATTCTGCGCGGGACGTCCTTCTGCTACGTCCC | |
| | TTCGGCCCTCAATCCAGCGGACCTTCCTTCCCGCGGCCTG | |
| | CTGCCGGCTCTGCGGCCTCTTCCGCCTCTTCGCCTTCGCC | |
| | CTCAGACGAGTCGGATCTCCCTTTGGGCCGCCTCCCCGCC | |
| | CATGTATCTTTTTCACCTGTGCCTTGTTTTTGCCTGTGTTC | |
| | CGCGTCCTACTTTTCAAGCCTCCAAGCTGTGCCTTGGGCG | |
| | GCTTTGGGGCATGGACATAGATCCCTATAAAGAATTTGG | |
| | TTCATCTTATCAGTTGTTGAATTTTCTTCCTTTGGAC | |
| 3′UTR | GCTGGAGCCTCGGTAGCCGTTCCTCCTGCCCGCTGGGCCT | 69 |
| (globin) | CCCAACGGGCCCTCCTCCCCTCCTTGCACCGGCCCTTCCT | |
| | GGTCTTTGAATAAA | |
| WPRE(r) | ATTCGAGCATCTTACCGCCATTTATTCCCATATTTGTTCTG | 70 |
| | TTTTTCTTGATTTGGGTATACATTTAAATGTTAATAAAAC | |
| | AAAATGGTGGGGCAATCATTTACATTTTTAGGGATATGTA | |
| | ATTACTAGTTCAGGTGTATTGCCACAAGACAAACATGTTA | |
| | AGAAACTTTCCCGTTATTTACGCTCTGTTCCTGTTAATCA | |
| | ACCTCTGGATTACAAAATTTGTGAAAGATTGACTGATATT | |
| | CTTAACTATGTTGCTCCTTTTACGCTGTGTGGATATGCTG | |
| | CTTTAATGCCTCTGTATCATGCTATTGCTTCCCGTACGGCT | |
| | TTCGTTTTCTCCTCCTTGTATAAATCCTGGTTGCTGTCTCT | |
| | TTATGAGGAGTTGTGGCCCGTTGTCCGTCAACGTGGCGTG | |
| | GTGTGCTCTGTGTTTGCTGACGCAACCCCCACTGGCTGGG | |
| | GCATTGCCACCACCTGTCAACTCCTTTCTGGGACTTTCGC | |
| | TTTCCCCCTCCCGATCGCCACGGCAGAACTCATCGCCGCC | |
| | TGCCTTGCCCGCTGCTGGACAGGGGCTAGGTTGCTGGGC | |
| | ACTGATAATTCCGTGGTGTTGTCGGGGAAGGGCC | |

In some embodiments, the rAAV virion genome comprises a polyadenylation (polyA) signal selected from Table 6. In some embodiments, the polyA signal comprises a polynucleotide sequence at least 75%, 80%, 85%, 90%, 92%, 93%, 94%, 95%, 96%, 97%, 98%, 99%, or 100% identical to any one of SEQ ID NOS 71-75.

TABLE 6

| POLY- | | SEQ |
|---|---|---|
| ADENYLATION | | ID |
| SITE | SEQUENCE | NO: |
| Rabbit globin | TGGCTAATAAAGGAAATTTATTTTCATTGCAATAGTGTG | 71 |
| (pAGlobin-Oc) | TTGGAATTTTTTGTGTCTCTCACTCGGAAGAACATATGG | |
| | GAGGGCAAATCATTTAAAACATCAGAATGAGTATTTGGT | |
| | TTAGAGTTTGGCAACATATGCCCATATGCTGGCTGCCAT | |
| | GAACAAAGGTTGGCTATAAAGAGGTCATCAGTATATGA | |
| | AACAGCCCCCTGCTGTCCATTCCTTATTCCATAGAAAAG | |
| | CCTTGACTTGAGGTTAGATTTTTTTTATATTTTGTTTTGTG | |
| | TTATTTTTTTCTTTAACATCCCTAAAATTTTCCTTACATGT | |
| | TTTACTAGCCAGATTTTTCCTCCTCTCCTGACTACTCCCA | |
| | GTCATAGCTGTCCCTCTTCTCTTATGGAGATC | |
| Bovine growth | TTGCCAGCCATCTGTTGTTTGCCCCTCCCCCGTGCCTTCC | 72 |
| hormone (pAGH-Bt- | TTGACCCTGGAAGGTGCCACTCCCACTGTCCTTTCCTAA | |
| version 1) | TAAAATGAGGAAATTGCATCGCATTGTCTGAGTAGGTGT | |
| | CATTCTATTCTGGGGGGTGGGGTGGGGCAGGACAGCAA | |
| | GGGGGAGGATTGGGAATACAATAGCAGGCATGCTGGGG | |
| | ATGCGGTGGGCTCTATGGGTACCCAGGTGCTGAAGAATT | |
| | GACCCGGTTCCTCCTGGG | |
| Bovine growth | TTGCCAGCCATCTGTTGTTTGCCCCTCCCCCGTGCCTTCC | 73 |
| hormone (pAGH-Bt- | TTGACCCTGGAAGGTGCCACTCCCACTGTCCTTTCCTAA | |
| version 2) | TAAAATGAGGAAATTGCATCGCATTGTCTGAGTAGGTGT | |
| | CATTCTATTCTGGGGGGTGGGGTGGGGCAGGACAGCAA | |
| | GGGGGAGGATTGGGAAGACAATAGCAGGCATGCTGGGG | |
| | ATGCGGTGGGCTCTATGGGTACCCAGGTGCTGAAGAATT | |
| | GACCCGGTTCCTCCTGGG | |
| Bovine growth | CTGTGCCTTCTAGTTGCCAGCCATCTGTTGTTTGCCCCTC | 74 |
| hormone (pAGH-Bt- | CCCCGTGCCTTCCTTGACCCTGGAAGGTGCCACTCCCAC | |
| version 3) | TGTCCTTTCCTAATAAAATGAGGAAATTGCATCGCATTG | |
| | TCTGAGTAGGTGTCATTCTATTCTGGGGGGTGGGGTGGG | |
| | GCAGGACAGCAAGGGGGAGGATTGGGAAGACAATAGC | |
| | AGGCATGCTGGGGATGCGGTGGGCTCTATGG | |
| Human growth | CTGCCCGGGTGGCATCCCTGTGACCCCTCCCCAGTGCCT | 75 |
| hormone (pAGH-Hs) | CTCCTGGCCCTGGAAGTTGCCACTCCAGTGCCCACCAGC | |
| | CTTGTCCTAATAAAATTAAGTTGCATCATTTTGTCTGACT | |
| | AGGTGTCCTTCTATAATATTATGGGGTGGAGGGGGGTGG | |
| | TATGGAGCAAGGGGCCCAAGTTGGGAAGAAACCTGTAG | |
| | GGCCTGC | |

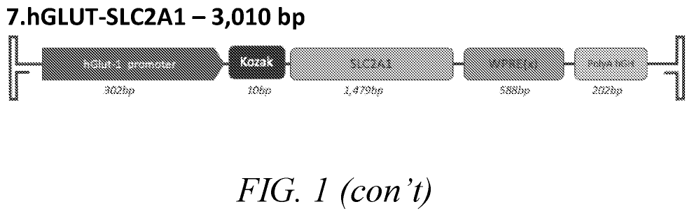

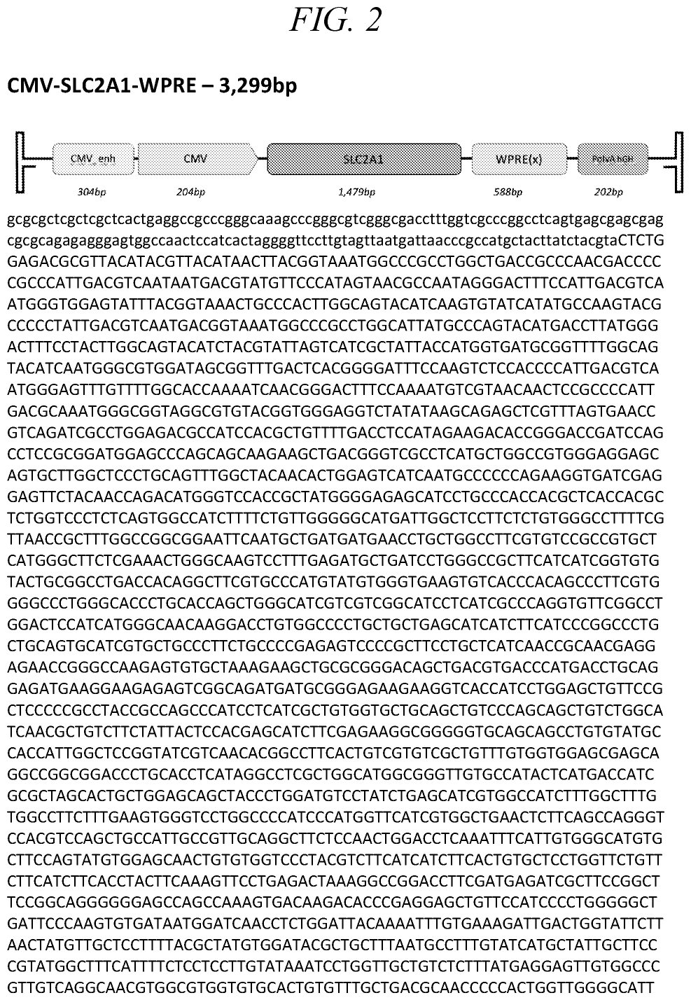

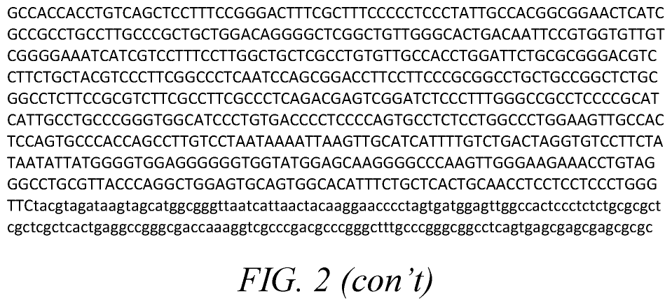

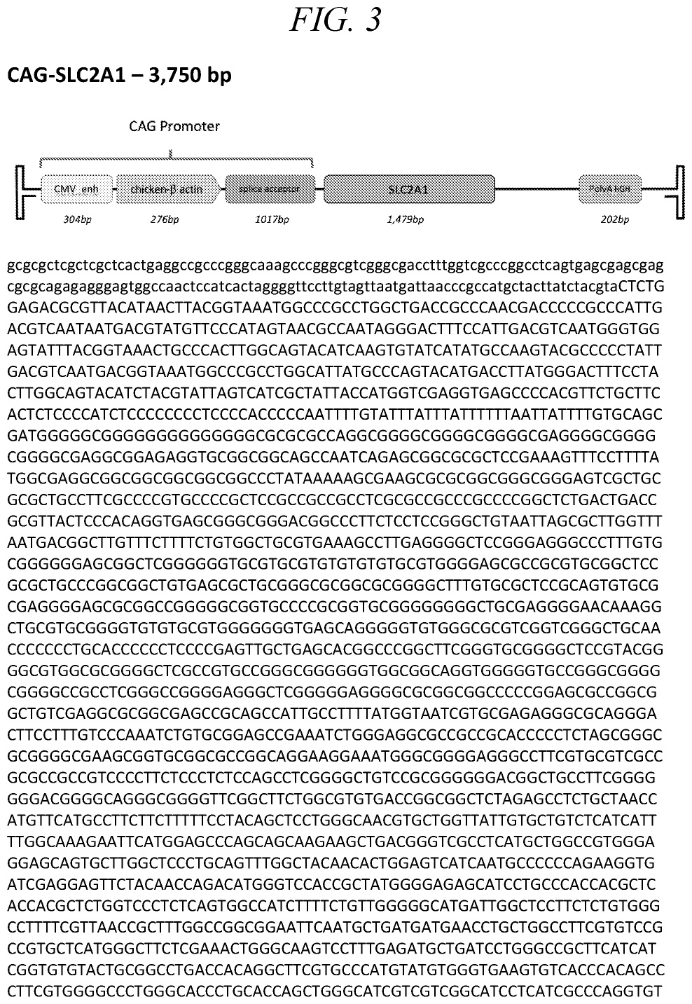

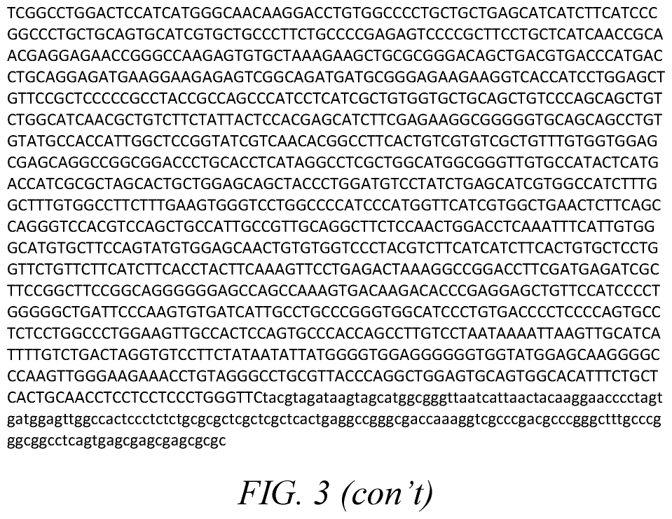

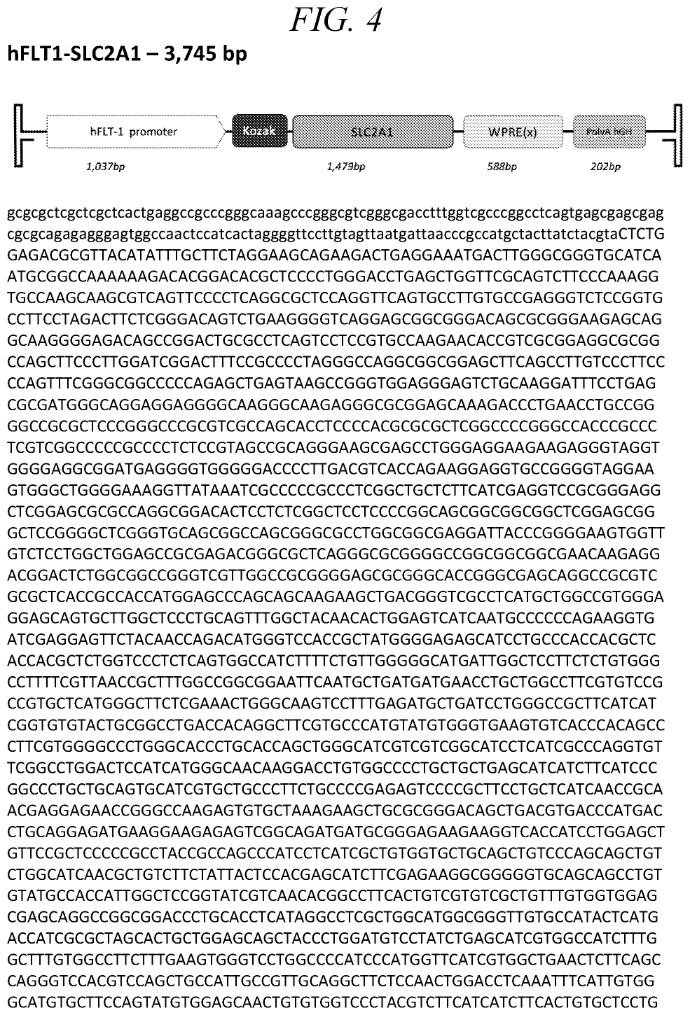

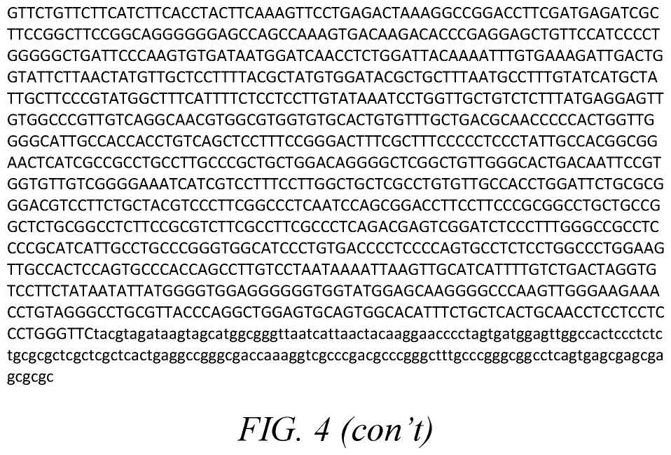

Illustrative rAAV virion genomes are depicted in FIG. 2-8 and provided as SEQ ID NOs: 17-25. The capitalized portion of each sequence is the expression cassette (SEQ TD NOs: 8-16, SEQ TD NO: 97, SEQ TD NO: 99, and SEQ TD NO: 101). In some embodiments, the rAAV virion genome comprises, consists essentially of, or consists of a polynucleotide sequence that shares at least 90%, at least 91%, at least 92%, at least 93%, at least 94%, at least 95%, at least 96%, at least 97%, at least 98%, at least 99%, or 100% identity to any one of SEQ TD NOs: 8-16, SEQ TD NO: 97, SEQ TD NO: 99, and SEQ TD NO: 101, optionally with or without the ITR sequences in lowercase. The coding sequence is underlined. The expression cassette is capitalized.

### Adeno-Associated Virus Genome

Adeno-associated virus (AAV) is a replication-deficient parvovirus, the single-stranded DNA genome of which is about 4.7 kb in length including two ˜145-nucleotide inverted terminal repeat (ITRs). There are multiple known variants of AAV, also sometimes called serotypes when classified by antigenic epitopes. The nucleotide sequences of the genomes of the AAV serotypes are known. For example, the complete genome of AAV-1 is provided in Genflank Accession No. NC_002077; the complete genome of AAV-2 is provided in Genflank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). The sequence of the AAVrh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep78, rep68, rep52, and rep40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vehicle for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate rAAV vector genomes, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Recombinant AAV virions useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such rAAV virions may be produced by techniques known in the art, e.g., by transfecting packaging cells or by transient transfection with helper plasmids or viruses. Typical examples of virus packaging cells include but are not limited to HeLa cells, SF9 cells (optionally with a baculovirus helper vector), 293 cells, etc. A Herpesvirus-based system can be used to produce AAV vectors, as described in US20170218395A1. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

AAV vectors useful in the practice of the present invention can be packaged into AAV virions (viral particles) using various systems including adenovirus-based and helper-free systems. Standard methods in AAV biology include those described in Kwon and Schaffer. Pharm Res. 25(3):489-99 (2008); Wu et al. Mol. Ther. 14(3):316-27 (2006); Burger et al. Mol. Ther. 10(2):302-17 (2004); Grimm et al. Curr Gene Ther. 3(4):281-304 (2003); Deyle D R, Russell D W. Curr Opin Mol Ther. 11(4):442-447 (2009); McCarty et al. Gene Ther. 8(16):1248-54 (2001); and Duan et al. Mol Ther. 4(4):383-91 (2001). Helper-free systems included those described in U.S. Pat. Nos. 6,004,797; 7,588,772; and 7,094,604.

AAV DNA in the rAAV vector genomes may be from any AAV variant or serotype for which a recombinant virus can be derived including, but not limited to, AAV variants or serotypes AAV1, AAV2, AAV3, AAV4, AAV5, AAV6, AAV7, AAV8, AAV9, AAV10, AAV11, AAV12, and AAV13. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In some cases, the rAAV comprises a self-complementary genome. As defined herein, an rAAV comprising a “self-complementary” or “double stranded” genome refers to an rAAV which has been engineered such that the coding region of the rAAV is configured to form an intra-molecular double-stranded DNA template, as described in McCarty et al. Self-complementary recombinant adeno-associated virus (scAAV) vectors promote efficient transduction independently of DNA synthesis. Gene Therapy. 8 (16): 1248-54 (2001). The present disclosure contemplates the use, in some cases, of an rAAV comprising a self-complementary genome because upon infection (such transduction), rather than waiting for cell mediated synthesis of the second strand of the rAAV vector genome, the two complementary halves of scAAV will associate to form one double stranded DNA (dsDNA) unit that is ready for immediate replication and transcription. It will be understood that instead of the full coding capacity found in rAAV (4.7-6 kb), rAAV comprising a self-complementary genome can only hold about half of that amount (≈2.4 kb).

In other cases, the rAAV vector genome is a single stranded genome. As defined herein, a “single standard” genome refers to a genome that is not self-complementary. In most cases, non-recombinant AAVs have singled stranded DNA genomes. There have been some indications that rAAVs should be scAAVs to achieve efficient transduction of cells. The present disclosure contemplates, however, rAAV vector genomes may have singled stranded genomes, rather than self-complementary genomes, with the understanding that other genetic modifications of the rAAV vector genome may be beneficial to obtain optimal gene transcription in target cells. In some cases, the present disclosure relates to single-stranded rAAV vector genomes capable of achieving efficient gene transfer to anterior segment in the mouse eye. See Wang et al. Single stranded adeno-associated virus achieves efficient gene transfer to anterior segment in the mouse eye. PLoS ONE 12(8): e0182473 (2017).

In preferred embodiments, the AAV is an AAV variant having a high degree of tropism for endothelial cells, such as the AAV2 variant BR1. Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). In some embodiments, said rAAV vector genome is of serotype AAV-BR1 and comprises a single-stranded genome. In some embodiments, a rAAV vector genome comprises the inverted terminal repeat (ITR) sequences of AAV2. In some embodiments, the rAAV vector genome comprises an AAV2 genome.

AAV genomes may comprise wild-type AAV sequence or they may comprise one or more modifications to a wild-type AAV sequence. In certain embodiments, an AAV genome comprises one or more amino acid modifications, e.g., substitutions, deletions, or insertions, within a capsid protein, e.g., VP1, VP2 and/or VP3. In particular embodiments, the modification provides for reduced immunogenicity when the AAV genome is provided to a subject.

Capsid proteins of a rAAV may be modified so that the rAAV is targeted to a particular target tissue of interest such as endothelial cells and/or endothelial tip cells. In some embodiments, the rAAV is directly injected into the intracerebroventricular space, injected intravenously or by combined intracerebroventicular and intravenous delivery to the subject.

In some embodiments, the rAAV virion is a variant of an AAV2 rAAV virion. The capsid many be an AAV2 capsid or functional variant thereof. The polypeptide sequence of the AAV2 VP1 is provided as SEQ ID NO: 76. In some embodiments, the AAV2 capsid shares at least 95% to a reference AAV2 capsid, e.g.,

(SEQ ID NO: 76)
MAADGYLPDWLEDTLSEGIRQWWKLKPGPPPPKPAERHKDDSRGLVLPGYKYLGPFNGLDKGEPVNEADAAALEH
DKAYDRQLDSGDNPYLKYNHADAEFQERLKEDTSFGGNLGRAVFQAKKRVLEPLGLVEEPVKTAPGKKRPVEHSP
VEPDSSSGTGKAGQQPARKRLNFGQTGDADSVPDPQPLGOPPAAPSGLGTNTMATGSGAPMADNNEGADGVGNSS
GNWHCDSTWMGDRVITTSTRTWALPTYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEYSWTGATKYHLNGRDSLVNPGPAMAS
HKDDEEKFFPQSGVLIFGKQGSEKTNVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKNTPVPANPSTTFSAAKFASFITQYST
GQVSVEIEWELQKENSKRWNPEIQYTSNYNKSVNVDFTVDTNGVYSEPRPIGTRYLTRNL

In some embodiments, the capsid protein is encoded by a polynucleotide supplied on a plasmid in trans to the transfer plasmid. In some embodiments, the polynucleotide sequence of AAV-BR1 cap shares at least 98%, at least 99%, or 100% identity SEQ ID NO: 103:

(SEQ ID NO: 103)
ATGGCTGCCGATGGTTATCTTCCAGATTGGCTCGAGGACACTCTCTCTGAAGGAATAAGACAGTGGTGGAAGCTC
AAACCTGGCCCACCACCACCAAAGCCCGCAGAGCGGCATAAGGACGACAGCAGGGGTCTTGTGCTTCCTGGGTAC
AAGTACCTCGGACCCTTCAACGGACTCGACAAGGGAGAGCCGGTCAACGAGGCAGACGCCGCGGCCCTCGAGCAC
GACAAAGCCTACGACCGGCAGCTCGACAGCGGAGACAACCCGTACCTCAAGTACAACCACGCCGACGCGGAGTTT
CAGGAGCGCCTTAAAGAAGATACGTCTTTTGGGGGCAACCTCGGACGAGCAGTCTTCCAGGCGAAAAAGAGGGTT
CTTGAACCTCTGGGCCTGGTTGAGGAACCTGTTAAGACGGCTCCGGGAAAAAAGAGGCCGGTAGAGCACTCTCCT
GTGGAGCCAGACTCCTCCTCGGGAACCGGAAAGGCGGGCCAGCAGCCTGCAAGAAAAAGATTGAATTTTGGTCAG
ACTGGAGACGCAGACTCAGTACCTGACCCCCAGCCTCTCGGACAGCCACCAGCAGCCCCCTCTGGTCTGGGAACT
AATACGATGGCTACAGGCAGTGGCGCACCAATGGCAGACAATAACGAGGGCGCCGACGGAGTGGGTAATTCCTCG
GGAAATTGGCATTGCGATTCCACATGGATGGGCGACAGAGTCATCACCACCAGCACCCGAACCTGGGCCCTGCCC
ACCTACAACAACCACCTCTACAAACAAATTTCCAGCCAATCAGGAGCCTCGAACGACAATCACTACTTTGGCTAC
AGCACCCCTTGGGGGTATTTTGACTTCAACAGATTCCACTGCCACTTTTCACCACGTGACTGGCAAAGACTCATC
AACAACAACTGGGGATTCCGACCCAAGAGACTCAACTTCAAGCTCTTTAACATTCAAGTCAAAGAGGTCACGCAG
AATGACGGTACGACGACGATTGCCAATAACCTTACCAGCACGGTTCAGGTGTTTACTGACTCGGAGTACCAGCTC
CCGTACGTCCTCGGCTCGGCGCATCAAGGATGCCTCCCGCCGTTCCCAGCAGACGTCTTCATGGTGCCACAGTAT
GGATACCTCACCCTGAACAACGGGAGTCAGGCAGTAGGACGCTCTTCATTTTACTGCCTGGAGTACTTTCCTTCT
CAGATGCTGCGTACCGGAAACAACTTTACCTTCAGCTACACTTTTGAGGACGTTCCTTTCCACAGCAGCTACGCT
CACAGCCAGAGTCTGGACCGTCTCATGAATCCTCTCATCGACCAGTACCTGTATTACTTGAGCAGAACAAACACT
CCAAGTGGAACCACCACGCAGTCAAGGCTTCAGTTTTCTCAGGCCGGAGCGAGTGACATTCGGGACCAGTCTAGG
AACTGGCTTCCTGGACCCTGTTACCGCCAGCAGCGAGTATCAAAGACATCTGCGGATAACAACAACAGTGAATAC
TCGTGGACTGGAGCTACCAAGTACCACCTCAATGGCAGAGACTCTCTGGTGAATCCGGGCCCGGCCATGGCAAGC
CACAAGGACGATGAAGAAAAGTTTTTTCCTCAGAGCGGGGTTCTCATCTTTGGGAAGCAAGGCTCAGAGAAAACA
AATGTGGACATTGAAAAGGTCATGATTACAGACGAAGAGGAAATCAGGACAACCAATCCCGTGGCTACGGAGCAG
TATGGTTCTGTATCTACCAACCTCCAGAGAGGCCAGAGAGGCAATCGGGGGACTGAGTGGGATGCCCAGGCGGCC
ACCGCAGATGTCAACACACAAGGCGTTCTTCCAGGCATGGTCTGGCAGGACAGAGATGTGTACCTTCAGGGGCCC
ATCTGGGCAAAGATTCCACACACGGACGGACATTTTCACCCCTCTCCCCTCATGGGTGGATTCGGACTTAAACAC
CCTCCTCCACAGATTCTCATCAAGAACACCCCGGTACCTGCGAATCCTTCGACCACCTTCAGTGCGGCAAAGTTT
GCTTCCTTCATCACACAGTACTCCACGGGACAGGTCAGCGTGGAGATCGAGTGGGAGCTGCAGAAGGAAAACAGC
AAACGCTGGAATCCCGAAATTCAGTACACTTCCAACTACAACAAGTCTGTTAATGTGGACTTTACTGTGGACACT
AATGGCGTGTATTCAGAGCCTCGCCCCATTGGCACCAGATACCTGACTCGTAATCTGTAA

In some embodiments, the disclosure further provides protein sequences for AAV-BR1 VP1 and functional variants thereof. The sequence of the AAV-BR1 VP1 polypeptide is provided as SEQ ID NO: 104. In some embodiments, the sequence of AAV VP1 shares at least 98%, at least 99%, or 100% identity SEQ ID NO: 104:

(SEQ ID NO: 104)
MAADGYLPDWLEDTLSEGIRQWWKLKPGPPPPKPAERHKDDSRGLVLPGYKYLGPFNGLDKGEPVNEADAAALEH
DKAYDRQLDSGDNPYLKYNHADAEFQERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGTNTMATGSGAPMADNNEGADGVGNSS
GNWHCDSTWMGDRVITTSTRTWALPTYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQLPYVLGSAHQGCLPPFPADVFMVPQY
GYLTLNNGSQAVGRSSFYCLEYFPSQMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEYSWTGATKYHLNGRDSLVNPGPAMAS
HKDDEEKFFPQSGVLIFGKQGSEKTNVDIEKVMITDEEEIRTTNPVATEQYGSVSTNLQRGQRGNRGTEWDAQAA
TADVNTQGVLPGMVWQDRDVYLQGPIWAKIPHTDGHFHPSPLMGGFGLKHPPPQILIKNTPVPANPSTTFSAAKF
ASFITQYSTGQVSVEIEWELQKENSKRWNPEIQYTSNYNKSVNVDFTVDTNGVYSEPRPIGTRYLTRNL

In some embodiments, the disclosure further provides protein sequences for AAV-BR1 VP2 and functional variants thereof. The sequence of the AAV-BR1 VP1 polypeptide is provided as SEQ ID NO: 105. In some embodiments, the sequence of AAV VP2 shares at least 98%, at least 99%, or 100% identity SEQ ID NO: 105:

(SEQ ID NO: 105)
MAPGKKRPVEHSPVEPDSSSGTGKAGQQPARKRLNFGQTGDADSVPDPQPLGOPPAAPSGLGTNTMATGSGAPMA
DNNEGADGVGNSSGNWHCDSTWMGDRVITTSTRTWALPTYNNHLYKQISSQSGASNDNHYFGYSTPWGYFDFNRF
HCHFSPRDWQRLINNNWGFRPKRLNFKLFNIQVKEVTQNDGTTTIANNLTSTVQVFTDSEYQLPYVLGSAHQGCL
PPFPADVFMVPQYGYLTLNNGSQAVGRSSFYCLEYFPSQMLRTGNNFTFSYTFEDVPFHSSYAHSQSLDRLMNPL
IDQYLYYLSRTNTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQYGSVSTNLQRGQ
RGNRGTEWDAQAATADVNTQGVLPGMVWQDRDVYLQGPIWAKIPHTDGHFHPSPLMGGFGLKHPPPQILIKNTPV
PANPSTTFSAAKFASFITQYSTGQVSVEIEWELQKENSKRWNPEIQYTSNYNKSVNVDFTVDTNGVYSEPRPIGT
RYLTRNL

In some embodiments, the disclosure further provides protein sequences for AAV-BR1 VP3 and functional variants thereof. The sequence of the AAV-BR1 VP1 polypeptide is provided as SEQ ID NO: 106. In some embodiments, the sequence of AAV VP3 shares at least 98%, at least 99%, or 100% identity SEQ ID NO: 106:

(SEQ ID NO: 106)
MATGSGAPMADNNEGADGVGNSSGNWHCDSTWMGDRVITTSTRTWALPTYNNHLYKQISSQSGASNDNHYFGYST
PWGYFDFNRFHCHFSPRDWQRLINNNWGFRPKRLNFKLFNIQVKEVTQNDGTTTIANNLTSTVQVFTDSEYQLPY
VLGSAHQGCLPPFPADVFMVPQYGYLTLNNGSQAVGRSSFYCLEYFPSQMLRTGNNFTFSYTFEDVPFHSSYAHS
QSLDRLMNPLIDQYLYYLSRTNTPSGTTTQSRLQFSQAGASDIRDQSRNWLPGPCYRQQRVSKTSADNNNSEYSW
TGATKYHLNGRDSLVNPGPAMASHKDDEEKFFPQSGVLIFGKQGSEKTNVDIEKVMITDEEEIRTTNPVATEQYG
SVSTNLQRGQRGNRGTEWDAQAATADVNTQGVLPGMVWQDRDVYLQGPIWAKIPHTDGHFHPSPLMGGFGLKHPP
PQILIKNTPVPANPSTTFSAAKFASFITQYSTGQVSVEIEWELQKENSKRWNPEIQYTSNYNKSVNVDFTVDTNG
VYSEPRPIGTRYLTRNL

In some embodiments, the capsid comprises an insertion in the (GH-1 loop, that is between approximately amino acid residue 588 and residue 589, in the AAV2 VP1 reference sequence set forth in SEQ ID NO 76. In some embodiments, the insertion sequence shares at least 98%, at least 99%, or 100% identity SEQ ID NO: 107:

(SEQ ID NO: 107)
XDGXXWX,

wherein each X is any amino acid.

In some embodiments, the capsid comprises an insertion in the GH loop between amino acids 588 and 589 compared to a VP1 reference sequence as set forth in SEQ ID NO 76. In some embodiments, the insertion sequence shares at least 98%, at least 99%, or 100% identity SEQ ID NO: 108:

(SEQ ID NO: 108)
ADGVQWT

In some embodiments, the capsid comprises an insertion in the GH loop between amino acids 588 and 589 compared to a VP1 reference sequence as set forth in SEQ ID NO 76. In some embodiments, the insertion sequence shares at least 98%, at least 99%, or 100% identity SEQ ID NO: 109:

(SEQ ID NO: 109)
DDGVSWK

In some embodiments, the capsid comprises an insertion in the (G-1 loop between amino acids 588 and 589 compared to a VP1 reference sequence as set forth in SEQ ID NO 76. In some embodiments, the insertion sequence shares at least 98%, at least 99%, or 100% identity SEQ ID NO: 110:

(SEQ ID NO: 110)
SDGLTWS

In some embodiments, the capsid comprises an insertion in the (G1H loop between amino acids 588 and 589 compared to a VP1 reference sequence as set forth in SEQ ID NO 76. In some embodiments, the insertion sequence shares at least 98%, at least 99%, or 100% identity SEQ ID NO: 111:

(SEQ ID NO: 111)
SDGLAWV

In some embodiments, the capsid comprises an insertion in the GH loop between amino acids 588 and 589 compared to a VP1 reference sequence as set forth in SEQ ID NO 76. In some embodiments, the insertion sequence shares at least 98%, at least 99%, or 100% identity SEQ ID NO: 112:

(SEQ ID NO: 112)
NRGTEWD

Further AAV capsids used in the rAAV virions of the disclosure include those disclosed in Pat. Pub. Nos. WO 2009/012176 A2 and WO 2015/168666 A2.

Use of an AAV-BR1 may result in endothelial-cell specific expression of the Glut1 transgene and, accordingly, increased efficacy of the transgene.

In some embodiments, rAAV vector genome is not an AAV2 genome. Without being bound by theory, use of an AAV2 genome results in transduction of neuronal cells in addition to or instead of endothelial cells. Without being bound by theory, spread of AAV2 genome within the CNS and associated cerebrovasculature is limited by its interaction with Heparan Sulfate Proteoglycan (HSPG) receptors.

### Pharmaceutical Compositions and Kits

In an aspect, the disclosure provides pharmaceutical compositions comprising the rAAV virion of the disclosure and one or more pharmaceutically acceptable carriers, diluents, or excipients.

For purposes of administration, e.g., by injection, various solutions can be employed, such as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as Poloxamer 188, e.g., at least 0.001% or 0.01%. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include but are not limited to sterile aqueous solutions or dispersions and sterile powders for the extemporaneous preparation of sterile injectable solutions or dispersions. In all cases the form is sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions may be prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the methods of preparation are vacuum drying and the freeze-drying technique that yield a powder of the active ingredient plus any additional desired ingredient from the previously sterile-filtered solution thereof.

In another aspect, the disclosure comprises a kit comprising an rAAV virion of the disclosure and instructions for use.

### Methods of Use

In an aspect, the disclosure provides a method of increasing GLUT1 activity in a cell, comprising contacting the cell with an rAAV of the disclosure. In another aspect, the disclosure provides a method of increasing GLUT1 activity in a subject, comprising administering to an rAAV of the disclosure. In some embodiments, the cell and/or subject is deficient in SLC2A1 messenger RNA or GLUT1 protein expression levels and/or activity and/or comprises a loss-of-function mutation in SLC2A1. The cell may be an endothelial cell, e.g. an endothelial tip cell or a cerebral vascular endothelial cell.

In some embodiments, the method restores normal function of endothelial tip cells. In some embodiments, the method restores GLUT1 transporter protein expression levels in cell culture and/or in vivo. In some embodiments, the method restores normal glucose transport and metabolism (e.g. glycolysis, lactate production) in cell culture and/or in vivo. In some embodiments, the method restores normal angiogenesis and/or development of the microvasculature in central nervous system (CNS).

In some embodiments, the method restores normal function of cerebral vascular endothelial cells. In some embodiments, the method restores GLUT1 transporter protein expression levels in cell culture and/or in vivo. In some embodiments, the method restores normal glucose transport and metabolism (e.g. glycolysis, lactate production) in cell culture and/or in vivo. In some embodiments, the method restores normal angiogenesis and/or development of the microvasculature in central nervous system (CNS).

### Methods of Treatment

In another aspect, the disclosure provides a method of treating a disease or disorder in a subject in need thereof, comprising administering to the subject an effective amount of an rAAV virion of the disclosure. In some embodiments, the disease or disorder is a neurological disease or disorder. In some embodiments, the subject suffers from a genetic disruption in SLC2A1 expression or function. In some embodiments, the disease or disorder is a GLUT1 Deficiency Syndrome (GLUT1 DS).

The AAV-mediated delivery of GLUT1 protein to the vasculature of the CNS may increase life span, prevent, diminish, mitigate, or attenuate neuronal degeneration, early-onset seizures, delayed development, acquired microcephaly (decelerating head growth), complex movement disorders (spasticity, ataxia, dystonia), paroxysmal eye-head movements, and/or low lactate and/or glucose concentration in cerebrospinal fluid (hypoglycorrhachia). In some embodiments, the method provides treatment early in the course of disease, e.g., in a newborn, infant, or juvenile.

In some embodiments, the methods of disclosure result in an increase (e.g., an increase of about 5% to about 10%, about 10% to about 20%, about 20% to about 30%, about 30% to about 40%, about 40% to about 50%, about 50% to about 70%, or about 70% to about 100%) in wildtype GLUT1 protein expression in the subject. In certain embodiments, the methods of the disclosure result in an increase (e.g., an increase of about 5% to about 25%, about 25% to about 50%, about 50% to about 100%, or about 100% to about 200%) in the ratio of wildtype to mutant GLUT1 protein in the subject.

In some embodiments, the methods of disclosure result in an increase (e.g., an increase of about 5% to about 10%, about 10% to about 20%, about 20% to about 30%, about 30% to about 40%, about 40% to about 50%, about 50% to about 70%, or about 70% to about 100%) in wildtype GLUT1 protein expression in an endothelial cell. In certain embodiments, the methods of the disclosure result in an increase (e.g., an increase of about 5% to about 25%, about 25% to about 50%, about 50% to about 100%, or about 100% to about 200%) in the ratio of wildtype to mutant GLUT1 protein in an endothelial cell.

The methods disclosed herein may provide efficient biodistribution in the brain and/or the vasculature of CNS. They may result in sustained expression in all, or a substantial fraction of, endothelial cells (e.g., endothelial tip cells). Notably, the methods disclosed herein may provide long-lasting expression of GLUT1 protein throughout development and aging of the subject following rAAV virion administration.

Combination therapies are also contemplated by the invention. Combinations of methods of the invention with standard medical treatments (e.g., corticosteroids or topical pressure reducing medications) are specifically contemplated, as are combinations with novel therapies. In some cases, a subject may be treated with a steroid and/or combination of immune suppressing agents to prevent or to reduce an immune response to administration of a rAAV described herein.

A therapeutically effective amount of the rAAV vector genome, e.g. for intracerebroventricular (ICV), intra-cisterna magna (ICM) injection, or intravenous (IV) injection, is a dose of rAAV ranging from about 1×10¹² vg/kg to about 5×10¹² vg/kg, or about 1×10¹³ vg/kg to about 5×10¹³ vg/kg, or about 1×10¹⁴ vg/kg to about 5×10¹⁴ vg/kg, or about 1×10¹⁵ vg/kg to about 5×10¹⁵ vg/kg, by brain weight. The invention also comprises compositions comprising these ranges of rAAV vector genomes.

For example, in particular embodiments, a therapeutically effective amount of rAAV vector genome is a dose of about 1×10¹⁰ vg, about 2×10¹⁰ vg, about 3×10¹⁰ vg, about 4×10¹⁰ vg, about 5×10¹⁰ vg, about 6×10¹⁰ vg, about 7×10¹⁰ vg, about 8×10¹⁰ vg, about 9×10¹⁰ vg, about 1×10¹² vg, about 2×10¹² vg, about 3×10¹² vg, about 4×10¹² vg, about 4×10¹³ vg, and about 4×10¹⁴ vg. The invention also comprises compositions comprising these doses of rAAV vector genome.

In some embodiments, for example where ICV injection is performed, a therapeutically effective amount of rAAV vector genome is a dose in the range of 1×10¹⁰ vg/hemisphere to 2×10¹⁴ vg/hemisphere, or about 1×10¹⁰ vg/hemisphere, about 1×10¹¹ vg/hemisphere, about 1×10¹² vg/hemisphere, 1×10¹³ vg/hemisphere, or about 1×10¹⁴ vg/hemisphere. In some embodiments, for example where ICM injection is performed, a therapeutically effective amount of rAAV vector genome is a dose in the range of 2×10¹⁰ vg total to 2×10¹⁴ vg total, or about 2×10¹⁰ vg total, about 2×10¹¹ vg total, about 2×10¹² vg total, about 2×10¹³ vg total, or about 2×10¹⁴ vg total.

In some embodiments, the therapeutic composition comprises more than about 1×10⁹, 1×10¹⁰, or 1×10¹¹ genomes of the rAAV vector genome per volume of therapeutic composition injected. In embodiments cases, the therapeutic composition comprises more than approximately 1×10¹¹, 1×10¹², 1×10¹³, or 1×10¹⁴ genomes of the rAAV per mL. In certain embodiments, the therapeutic composition comprises less than about 1×10¹⁴, 1×10¹³ or 1×10¹² genomes of the rAAV per mL.

Evidence of functional improvement, clinical benefit or efficacy in patients may be assessed by the analysis of paroxysmal eye-head movements, surrogate markers of reduction in seizure frequency (generalized tonic clonic and myoclonic seizures), lactate and/or glucose concentration in cerebrospinal fluid (CSF), assessment of developmental delay, chorea, dystonia, and microcephaly. Measures in cognition, motor, speech and language function using standard disease rating scales, such as Columbia Neurological Score, Composite Intellectual Estimate, Adaptive Behavior Composite, verbal and nonverbal cognitive skills and visuomotor integration, and Six Minute Walk Test. Cognitive and Developmental Assessments including the Peabody Developmental Motor Scales 2^{nd} edition (PDMS-2) and Bayley Scales of Infant Development, 3d edition applied as appropriate to level of child's disability. Gross motor function measure (GFMF-88), Pediatric Evaluation of Disability Inventory (PEDI). These or similar scales, as well as patient-reported outcomes on quality of life such as Caregiver Global Impression of Change in Seizure Duration (CGICSD) on a 3-point scale (decrease, no change, or increase in average duration), Pediatric Quality of Life Inventory (PedsQL™) and Vineland Adaptive Behavior Scales-2nd may demonstrate improvements in components of the disease. Baseline and post treatment Brain magnetic resonance imaging may show improvements or normalized brain volume for age of patient compared to age-matched patient control data and historical data from GLUT1 Deficiency patients.

Clinical benefit could be observed as increase in life-span, meeting normal neurodevelopmental milestones, normalized glucose concentration in CSF, decreases in frequency or magnitude paroxysmal eye-head movements, decrease or absence of epileptic seizure activity (including myoclonic, clonic, generalized tonic-clonic and/or epileptic spasm), improvement in, or lack of development of complex movement disorders such as spasticity, dystonia, and/or ataxia, and improved or normal performance in Columbia Neurological Score and/or Six Minute Walk Test. Evidence of neuroprotective and/or neurorestorative effects may be evident on all of the prior mentioned metrics and/or on magnetic resonance imaging (MRI) by characterizing overall brain size, lack of microcephaly and/or cortical and/or cerebellar atrophy.

In some embodiments, method causes increased glucose uptake by cells compared to cells contacted with, or of cells of a subject administered, a vector comprising an endogenous Glut1 promoter or a ubiquitous promoter. In some cases, the increase is at least 5%, at least 10%, at least 15%, at least 20%, at least 25%, at least 30%, at least 40%, or at least 50%. In some cases, the increase is at least 1.1-fold, at least 1.2-fold, at least 1.3-fold, at least 1.4-fold, at least 1.5-fold, at least 1.6-fold, at least 1.7-fold, or at least 1.8-fold. The vector may be any vector disclosed herein. The cell may be an endothelial cell or a neuronal cell. For example, the method may increase glucose uptake by human cerebral microvasculature endothelial cells, either in vitro or in vivo.

### Administration of Compositions

Administration of an effective dose of the compositions may be by routes standard in the art including, but not limited to, intravenous, intracerebral, intrathecal, intracisternal, or intra-cerebroventricular administration. In some cases, administration comprises intravenous, intracerebral, intrathecal, intracisternal or intracerebroventricular injection. Administration may be performed by intrathecal injection with or without Trendelenberg tilting. Intracisterna magna (ICM) delivery may be achieved via catheter entry at the intrathecal (IT) space. Intracerebroventricular injection(s) may be achieved via magnetic resonance imaging (MRI) guided neurosurgical targeting.

In some embodiments, the disclosure provides for systemic administration of an effective dose of rAAV and compositions of the invention. For example, systemic administration may be administration into the circulatory system so that the entire body is affected. Systemic administration includes intravenous administration through injection or infusion.

In some embodiments, administration of rAAV of the present invention may be accomplished by using any physical method that will transport the rAAV recombinant vector into the target tissue of an animal. Administration includes, but is not limited to, injection into the central nervous system (CNS) or cerebrospinal fluid (CSF) and/or intravenous injection.

In some embodiments, the methods of the disclosure comprise intracerebroventricular, intracisterna magna, intrathecal, or intraparenchymal delivery. Infusion may be performed using specialized cannula, catheter, syringe/needle using an infusion pump. Optionally, targeting of the injection site may be accomplished with MRI-guided imaging. Administration may comprise delivery of an effective amount of the rAAV virion, or a pharmaceutical composition comprising the rAAV virion, to the CNS. These may be achieved, e.g., via unilateral intraventricular injection, bilateral intraventricular injection, intracisternal magna infusion with Trendelenburg tilting procedure, or intracisternal magna infusion without Trendelenburg tilting procedure, intrathecal infusion with Trendelenburg tilting procedure, or intrathecal infusion without Trendelenburg tilting procedure. The compositions of the disclosure may further be administered intravenously.

In some embodiments, the methods of the disclosure comprise administration of rAAV by intravenous (IV) injection. In some embodiments, the methods of the disclosure comprise administration of rAAV by intracerebroventricular (ICV) injection. In some embodiments, the methods of the disclosure comprise administration of rAAV by IV injection in combination with ICV injection.

Direct delivery to the CNS could involve targeting the intraventricular space, either unilaterally or bilaterally, specific neuronal regions or more general brain regions containing neuronal targets. Individual patient intraventricular space, brain region and/or neuronal target(s) selection and subsequent intraoperative delivery of AAV could by accomplished using a number of imaging techniques (MRI, CT, CT combined with MRI merging) and employing any number of software planning programs (e.g., Stealth System, Clearpoint Neuronavigation System, Brainlab, Neuroinspire etc). Intraventricular space or brain region targeting and delivery could involve use of standard stereotactic frames (Leksell, CRW) or using frameless approaches with or without intraoperative MRI. Actual delivery of AAV may be by injection through needle or cannulae with or without inner lumen lined with material to prevent adsorption of rAAV (e.g. Smartflow cannulae, MRI Interventions cannulae). Delivery device consists of syringe(s) and automated infusion or microinfusion pumps with preprogrammed infusion rates and volumes. A syringe/needle combination or just a guide cannulae for the needle may be interfaced directly with the stereotactic frame. Infusion may include constant flow rate or varying rates with convection enhanced delivery.

### EXAMPLES

### Example 1: Pre-Clinical Bioactivity and Efficacy

Recombinant AAV virions with an AAV-BR1 capsid are produced using the vector genomes disclosed in FIGS. 2-8. These are evaluated in mouse models of disease as a consequence of GLUT1 deficiency disease. One model employs a flox-ed GLUT1 gene crossed to a transgenic animal that expresses Cre lox from a constitutive promoter or an endothelial-specific promoter (e.g., Tie-2). The resulting mice are heterozygous null at the GLUT1 locus and exhibit a developmental phenotype that mimics human disease. A second mouse model of GLUT1 DS is a heterozygous haploinsufficient mouse generated by targeted disruption of the promoter and exon 1 regions of the mouse GLUT-1 gene (GLUT-1^{+/-} mice). Additional animal models may include a GLUT1 DS model where the GLUT1 gene has a S324P point mutation.

Gene expression and dose-response is evaluated in vitro (using endothelial and neuronal cell lines) and in vivo (using wild-type and GLUT1 DS model mice). Cultured cells (Human Embryonic Kidney cells 293, HEK293; human umbilical vein endothelial cells, HUVEC; human brain-derived endothelial cells, bEND3; human brain microvasculature endothelial cells, HBEC-5i; human brain microvascular endothelial cell line, hCMEC/D3 (Blood-Brain Barrier model); human glial oligodendrocytic hybrid cells, M03.13; human neuroblastoma, SH-SY5Y) transfected with SLC2A1 expression vectors will reveal transduction efficiency by quantitative real-time PCR analyses, GLUT1 levels by ELISA and/or Western blot. Proof of concept and efficacy of AAV vector construct(s) will be revealed in vivo using GLUT1 DS mice by expression of transgene (GLUT1 protein) in the CNS and CNS vasculature by immunolabeling, enhanced brain capillary density and/or increase in blood vessel size in CNS, increase in brain glucose uptake using positron emission tomography (PET), increase in CSF glucose levels or lactate levels and/or in CSF/blood glucose ratio, increase in CSF lactate levels, and improvement in motor performance using standard assays such as rotarod and/or vertical pole assay, relative to GLUT1 DS mutant mouse controls. Gene expression and efficacy in vivo using GLUT1 DS mouse model(s) will be evident following delivery of AAV vector construct(s) by intravenous or direct injection to the intracerebroventricular space, while employing these routes of administration either alone and/or in combination.

### Example 2: In Vitro Evaluation of Glut1 Expression Using Endothelial Promoters

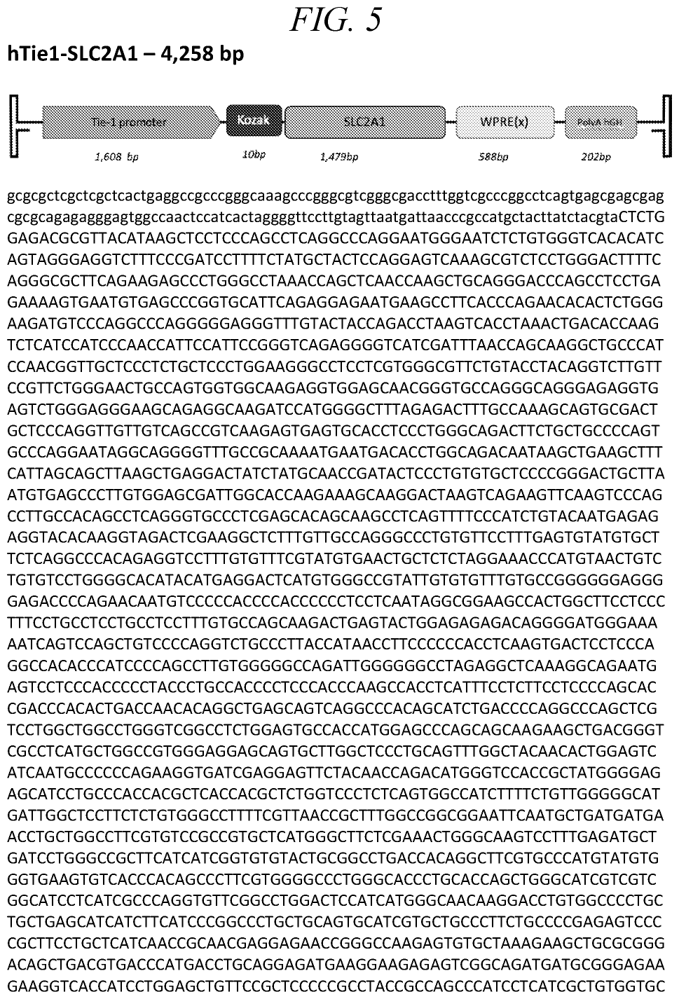

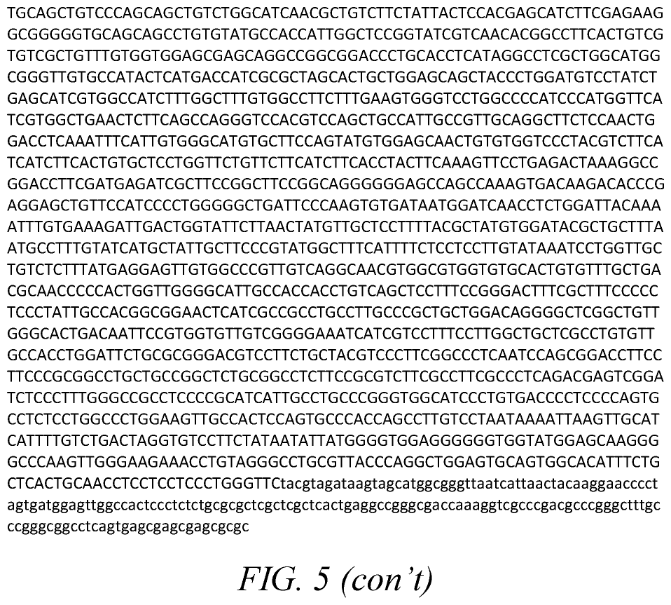

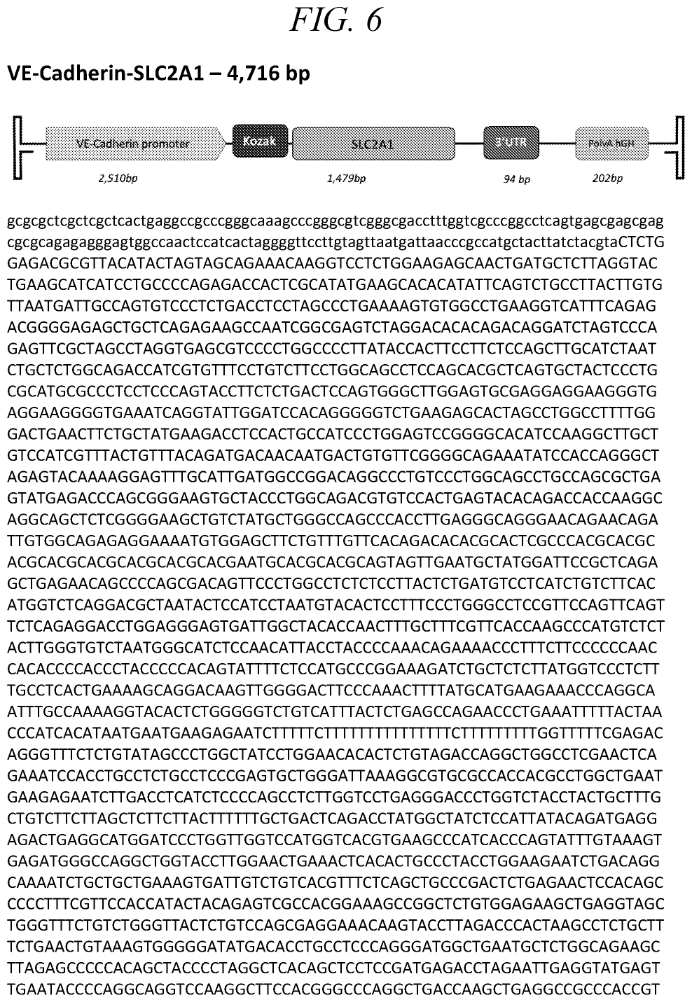

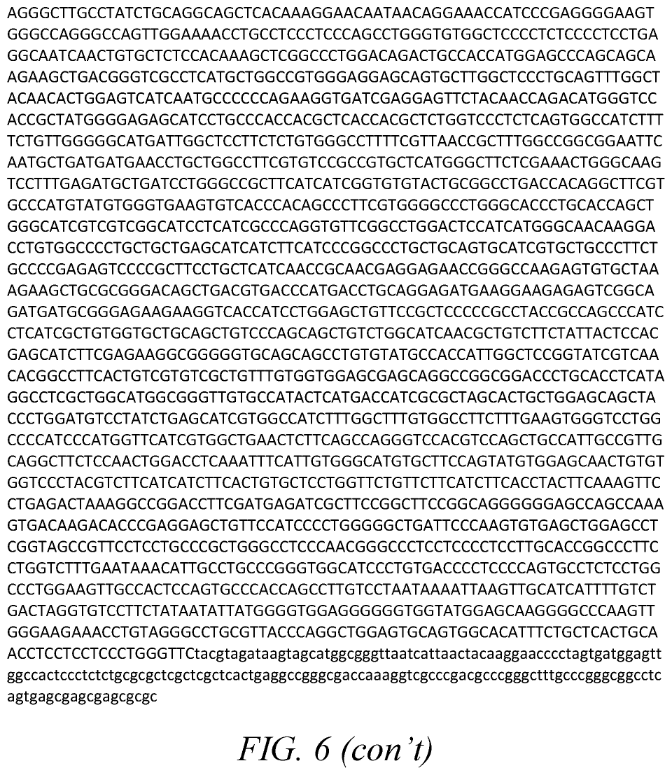

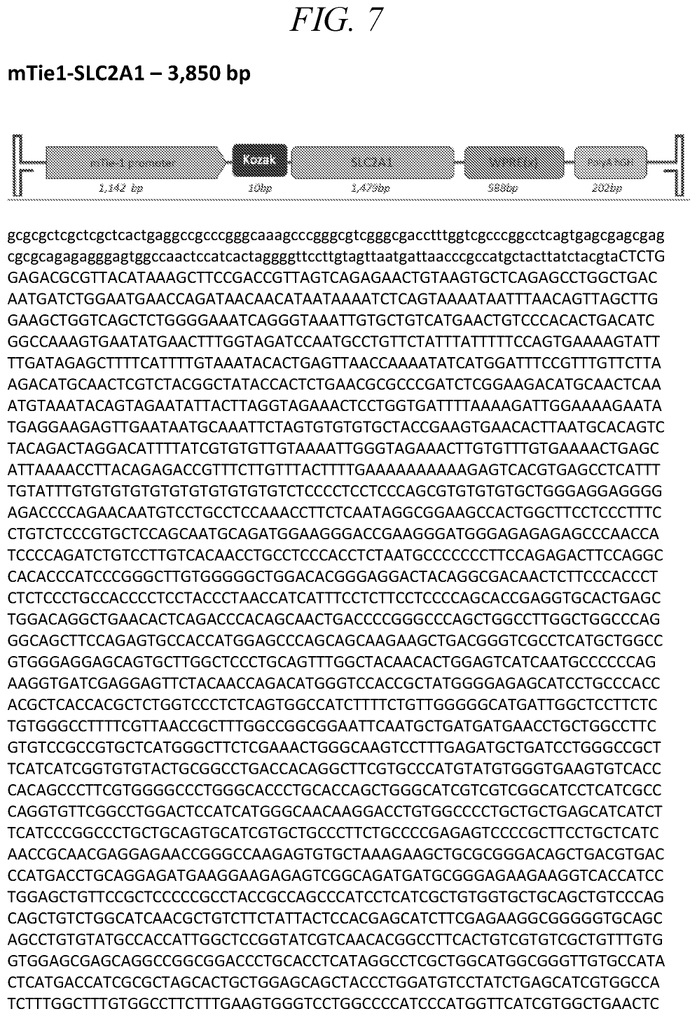

Gene expression was evaluated in vitro using human cerebral microvasculature endothelial cells (hCMEC/D3). Expression of Glut1 by hCMEC/D3 cells transfected with AAV9 vectors encoding SLC2A1 under the control of a hFLT1, mTIE1, hGlut1, or CMV promoter (diagramed in FIG. 10C) was evaluated (FIG. 9). Expression from the endothelial promoters (hFLT1 and mTIE1) was comparable to expression from the Glut1 promoter, and much lower than expression from the CMV promoter. A similar pattern of expression levels between these constructs was observed by immunofluorescence microscopy (FIG. 10A and FIG. 10B).

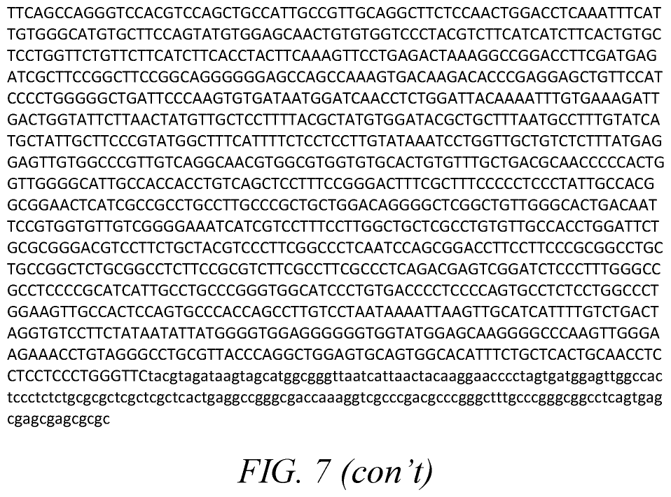

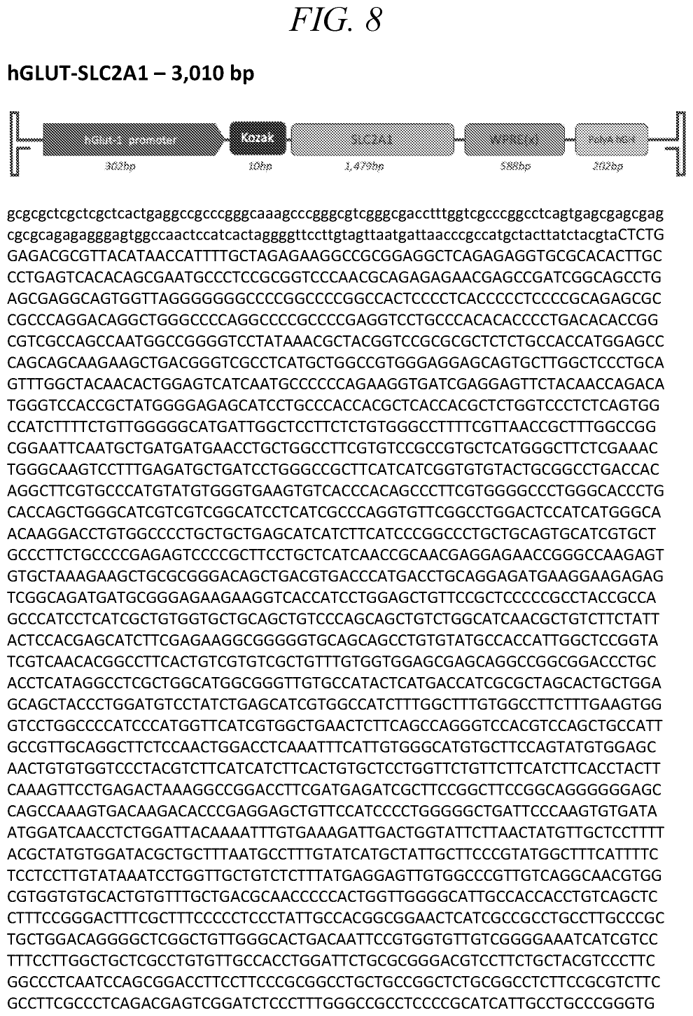

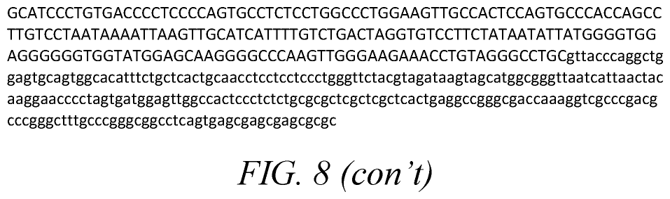

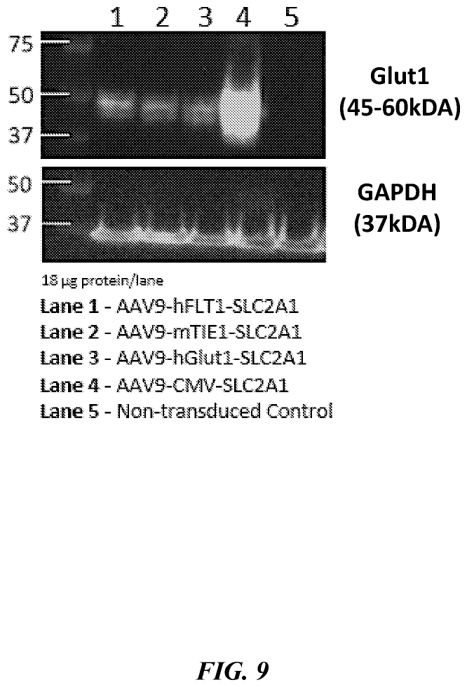

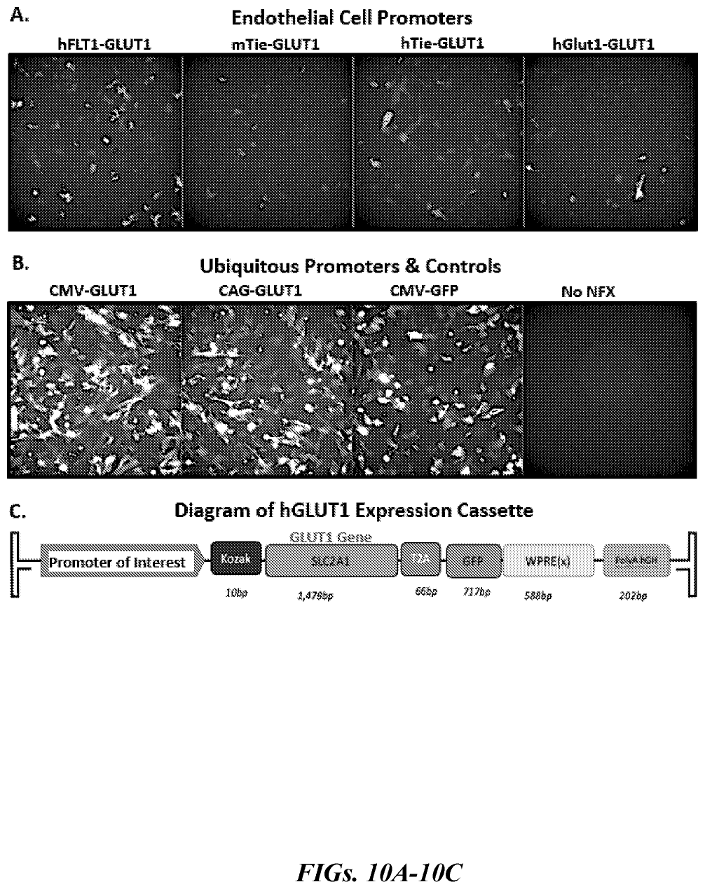

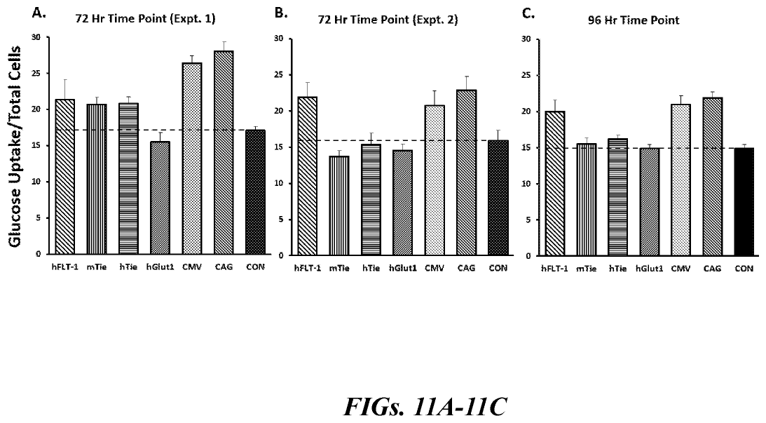

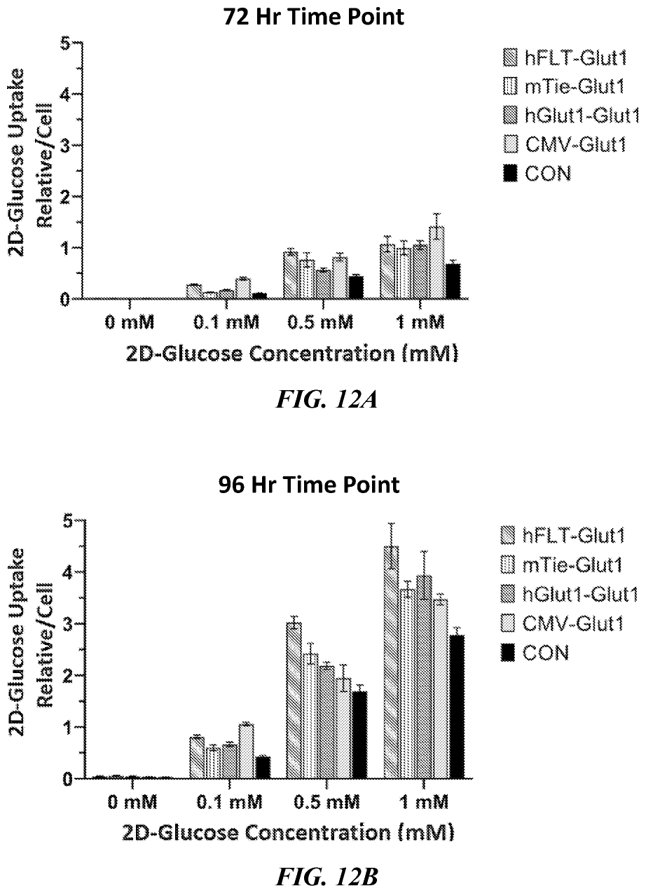

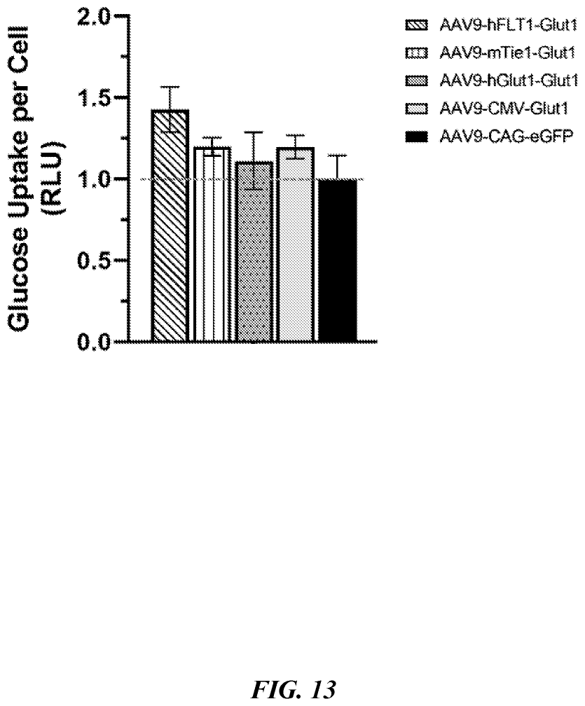

Surprisingly, 2-Deoxy-D-glucose (2-DG) uptake by human cerebral microvasculature endothelial cells transfected or transduced with the gene under the control of the endothelial promoters was greater than the control Glut1 promoter, with the hFLT-1 promoter demonstrating the highest level of 2-DG (glucose) uptake (FIGS. 11A-11C, FIG. 12, and FIG. 13). This finding of greater 2-DG (glucose) uptake with the hFLT-1 promoter construct was also observed across a range of 2-DG concentrations (FIG. 12A; 0, 0.1, 0.5, and 1 mM) and varying time points following transfection (FIG. 12B) and in some instances was found to be comparable or slightly greater than that observed with the CMV promoter (FIG. 11A-11C; FIG. 12A, 12B; FIG. 13).

FIG. 9 Expression of transgene protein (Glut1-GFP) following transfection of human cerebral microvasculature endothelial cells (hCMEC/d3s).

FIG. 10A. GFP fluorescence 72 hours following transfection with constructs containing one of several endothelial cell promoters driving expression of Glut1-GFP transgene.

FIG. 10B. GFP fluorescence 72 hours following transfection with constructs containing one of two ubiquitous promoters (CMV or CAG), control vector without Glut1 (CMV-GFP) or no transfection (No NFX). Images obtained using Operetta CLS™ (PerkinElmer®).

FIG. 10C. Diagram of expression cassette containing the promoter of interest (hFLT1, mTie, hTie or hGlut1) and the GLUT1 (SLC2A1) gene (T2A linked-GFP) and regulatory elements flanked by AAV2 inverted terminal repeats (ITRs).

FIGS. 11A-11C. 2-Deoxy-D-glucose (glucose) Uptake in hCMEC/d3 cells following expression of human GLUT1 (SLC2A1). Human cerebromicrovascular endothelial cells (hCMEC/d3s) were transfected with plasmids expressing either CAG-GFP (CON; negative control) or with a hGLUT1-t2A-eGFP transgene driven by one of several endothelial-specific promoters (i.e., hFLT1, mTie, hTie or hGlut1) or by the ubiquitous CMV or CAG promoters. Glucose uptake was measured using a luminescence-based kit (Promega®) with 0.5 mM 2-Deoxyglucose (2-DG) in culture media. Glucose (2-DG) uptake was normalized by Total Cells using phase-contrast imaging [Error bars represent S.E.M; n=6 replicates per condition].

FIG. 11A. Glucose (2-DG) uptake was measured at 72 hours post-transfection in a first experiment.

FIG. 11B. Glucose (2-DG) uptake was measured at 72 hours post-transfection in a second experiment.

FIG. 11C. Glucose (2-DG) uptake was measured at 96 hours post-transfection.

FIG. 12A. shows glucose (2-DG) uptake in hCMEC/D3 cells following expression of human Glut1 (SLC2A1) at a 72-hour time point.

FIG. 12B. shows glucose (2-DG) uptake in hCMEC/D3 cells following expression of human Glut1 (SLC2A1) at a 96-hour time point.

FIG. 13. 2-Deoxy-D-glucose (glucose) Uptake Following AAV9-mediated Expression of hGLUT1 (SLC2A1) in hCMEC/D3 cells. Human cerebromicrovascular endothelial cells (hCMEC/d3s) were transduced with AAV9 vectors (3×10⁵ vector genomes/cell) expressing either CAG-GFP (negative control) or the hGLUT1 transgene driven by one of several endothelial-specific promoters (i.e., hFLT1, mTie1 or hGlut1) or by the ubiquitous CMV promoter. Glucose (2-DG) uptake was measured 72 hours post-transduction using the luminescence-based Glucose Uptake-Glo kit (Promega®) and normalized per cell using the RealTime-Glo MT Cell Viability Assay (Promega®) [Error bars represent S.E.M; n=4 replicates per condition].

### Example 3: In Vivo Evaluation of AAV-BR1 Capsid-Mediated Glut1 Expression Using Endothelial Promoters in an Animal Model of Glut1 Deficiency

A series of experiments evaluating the in vivo effects of AAV-BR1 capsid-mediated expression of Glut1 transporter protein in the mouse model of GLUT1 Deficiency Syndrome (DS) will be performed. This model employs a mouse that is heterozygous haploinsufficient due to a targeted disruption of the promoter and exon 1 regions of the mouse GLUT-1 gene (GLUT-1+/− mice) and displays the characteristic features of human GLUT DS such as seizure activity, hypoglycorrhachia, microcephaly and impairments in motor function (Wang et al, Hum Mol Gen, 2006; Tang et al., Nat Comm, 2016). AAV-BR1 virions will be evaluated at different doses and different routes of administration (intravenous or intracerebroventricular) with expression of the GLUT1 transgene driven by either a ubiquitous promoter (CMV) or one of several endothelial cell promoters (e.g., hFLT-1). The extent to which endothelial cell promoter-mediated GLUT1 transgene expression following delivery using an AAV-BR1 virion can prevent or mitigate the functional and pathological deficits in this mouse model will be evaluated. Potential beneficial effects of AAV-BR1-mediated Glut1 protein expression when administered to the heterozygous haploinsufficient mouse will be revealed by comparisons to untreated GLUT-1+/− control mice and consist of improved or normalized body weight gain, behavioral performance on motor tests (e.g. rotarod, vertical pole assay), CSF glucose levels, brain weight, and integrity and size of brain microvasculature (e.g. brain capillary density, vessel size, number of vessel branch points).

1. A recombinant adeno-associated virus (rAAV) virion, comprising a vector genome and a capsid,
wherein the vector genome comprises an expression cassette, flanked by 5′ and 3′ inverted terminal repeats (ITRs),
wherein the expression cassette comprises a polynucleotide sequence encoding GLUT1 or a functional variant thereof, operatively linked to a promoter, and
wherein the capsid is a BR1 capsid or a functional variant thereof.

2. (canceled)

3. The rAAV virion of claim 1, wherein the capsid comprises the polypeptide sequence motif XDGXXWX, wherein each X is any amino acid (SEQ ID NO: 107).

4. The rAAV virion of claim 1, wherein the capsid comprises the polypeptide sequence ADGVQWT (SEQ ID NO:108), DDGVSWK (SEQ ID NO:109), SDGLTWS (SEQ ID NO:110), or SDGLAWV (SEQ ID NO:111).

5. The rAAV virion of claim 1, wherein the capsid comprises the polypeptide sequence NRGTEWD (SEQ ID NO: 112) or a functional variant having 1, 2, 3, or more substitutions thereto.

6. (canceled)

7. The rAAV virion of claim 1, wherein the capsid comprises an insertion of the polypeptide sequence NRGTEWD (SEQ ID NO: 112) in the GH loop compared to an AAV2 VP1 reference sequence as set forth in SEQ ID NO 76.

8. The rAAV virion of claim 1, wherein the capsid comprises: (a) a VP3 polypeptide that shares at least 98%, at least 99%, or 100% identity to an AAV-BR1 VP3 polypeptide sequence as set forth in SEQ ID NO:106; (b) a VP2 polypeptide that shares at least 98%, at least 99%, or 100% identity to an AAV-BR1 VP2 polypeptide sequence as set forth in SEQ ID NO:105; (c) a VP1 polypeptide that shares at least 98%, at least 99%, or 100% identity to an AAV-BR1 VP1 polypeptide sequence as set forth in SEQ ID NO: 104; or (d) any combination of (a)-(c).

9-11. (canceled)

12. The rAAV virion of claim 1, wherein the promoter is a FLT-1 promoter; and wherein: (a) the FLT-1 promoter is a human FLT-1 (hFLT-1) promoter; or (b) the hFLT-1 promoter shares at least 75%, at least 80%, at least 85%, at least 90%, at least 95%, at least 96%, at least 97%, at least 98%, at least 99% or 100% identity with SEQ ID NO: 1.

13. (canceled)

14. The rAAV virion of claim 1, wherein the expression cassette comprises: (a) a human growth hormone (hGH) polyA; (b) a Woodchuck Hepatitis Virus Posttranscriptional Regulatory Element (WPRE); (c) a 3′ untranslated region (3′ UTR) comprising a sequence that shares at least 75%, at least 80%, at least 85%, at least 90%, at least 95%, at least 96%, at least 97%, at least 98%, at least 99% or 100% identity with SEQ ID NO: 4; or (d) any combination of (a)-(c).

15-16. (canceled)

17. The rAAV virion of claim 1, wherein: (a) the polynucleotide sequence encoding GLUT1 is a human SLC2A1 polynucleotide; or (b) the polynucleotide sequence encoding GLUT1 shares at least 75%, at least 80%, at least 85%, at least 90%, at least 95%, at least 96%, at least 97%, at least 98%, at least 99% or 100% identity with SEQ ID NO: 5.

18-19. (canceled)

20. The rAAV virion of claim 1, wherein the 5′ and 3′ inverted terminal repeats (ITRs) are: (a) AAV2 ITRs, or (b) an ITR that shares at least 75%, at least 80%, at least 85%, at least 90%, at least 95%, at least 96%, at least 97%, at least 98%, at least 99% or 100% identity with SEQ ID NO: 6 or SEQ ID NO: 7.

21. The rAAV virion claim 1, wherein the expression cassette shares at least 75%, at least 80%, at least 85%, at least 90%, at least 95%, at least 96%, at least 97%, at least 98%, at least 99% or 100% identity with any one of SEQ ID NOs: 8-16, SEQ ID NO: 97, SEQ ID NO: 99, and SEQ ID NO: 101.

22. The rAAV virion of claim 21, wherein the rAAV virion is not an AAV2 virion.

23. A method of treating and/or preventing a disease or disorder in a subject in need thereof, comprising administering the rAAV virion of claim 21 to the subject.

24. The method of claim 23, wherein the disease or disorder is a neurological disorder.

25. The method of claim 24, wherein the disease or disorder is Glucose transporter 1 deficiency syndrome (GLUT1 DS) or De Vivo Disease.

26. The method of claim 23, wherein the rAAV virion is administered by: (a) intracerebroventricular (ICV) injection; (b) intravenous (IV) injection; or (c) a combination of (a) and (b).

27-28. (canceled)

29. The method of claim 23, wherein the administration results in: (a) expression of the polynucleotide sequence encoding GLUT1 in the brain, (b) expression of the polynucleotide sequence encoding GLUT1 in the brain at increased levels compared to a reference rAAV virion; (c) an increase in expression of GLUT1 protein in the brain; (d) an increase in expression of GLUT1 protein in the brain compared to a reference AAV2 virion or a reference AAV9 virion; (e) an increase in expression of GLUT1 protein in the brain of at least about 10%, at least about 20%, at least about 30%, at least about 40%, at least about 50%, at least about 60%, at least about 70%, at least about 80%, at least about 90%, or 100%; (f) an increase in glucose levels in the CSF; (g) an increase in glucose levels in the CSF compared to a reference AAV2 virion or a reference AAV9 virion; (h) an increase in glucose levels in the CSF of at least about 10%, at least about 20%, at least about 30%, at least about 40%, at least about 50%, at least about 60%, at least about 70%, at least about 80%, at least about 90%, or 100%; (i) an increase in lactate levels in the CSF; (j) an increase in lactate levels in the CSF compared to a reference AAV2 virion or a reference AAV9 virion; (k) an increase in lactate levels in the CSF of at least about 10%, at least about 20%, at least about 30%, at least about 40%, at least about 50%, at least about 60%, at least about 70%, at least about 80%, at least about 90%, or 100%.

30-32. (canceled)

33. The method of claim 23, wherein the rAAV virion is administered at a dose of 1×10¹² vector genomes (vg), 1×10¹³ vg, 1×10¹⁴ vg, or 3×10¹⁴ vg.

34. (canceled)

35. A method of expressing GLUT1 in a cell, comprising contacting the cells with the rAAV virion of claim 1.

36. The method of claim 35, wherein the cell is: (a) an endothelial cell; (b) a cerebral microvasculature endothelial cell; (c) an in vivo endothelial cell; (d) a neuron; or (e) an in vivo neuron.

37-40. (canceled)

41. The method of claim 36, wherein the method comprises in vivo administration of the rAAV virion to a subject.

42. (canceled)

43. A pharmaceutical composition comprising the rAAV virion of claim 1.

44. A kit comprising the rAAV virion of claim 1 and instructions for use.
